(19) United States
(12) Patent Application Publication
(10) Pub. No.: US 2005/0281783 A1
(43) Pub. Date: Dec. 22, 2005

(54) Listeria-based EphA2 vaccines

(76) Inventors: Michael S. Kinch, Laytonsville, MD (US); Peter A. Kiener, Potomac, MD (US); Elizabeth Bruckheimer, Rockville, MD (US); Thomas W. Dubensky JR., Piedmont, CA (US); David N. Cook, Lafayette, CA (US)

Correspondence Address: JONES DAY, 222 EAST 41ST ST, NEW YORK, NY, 10017, US

(21) Appl. No.: 10/966,483

(22) Filed: Oct. 15, 2004

Related U.S. Application Data
(60) Provisional application No. 60/511,919, filed on Oct. 15, 2003.
(60) Provisional application No. 60/511,719, filed on Oct. 15, 2003.
(60) Provisional application No. 60/532,666, filed on Dec. 24, 2003.
(60) Provisional application No. 60/556,631, filed on Mar. 26, 2004.
(60) Provisional application No. 60/615,470, filed on Oct. 1, 2004.
(60) Provisional application No. 60/617,544, filed on Oct. 7, 2004.

(51) Int. Cl. A61K 48/00; C12N 1/21

(52) U.S. Cl. 424/93.2; 435/252.3

(57) ABSTRACT

The present invention relates to methods and compositions designed for the treatment, management, or prevention of cancer, particularly metastatic cancer and cancers of T cell origin, and hyperproliferative diseases involving EphA2-expressing cells. The methods of the invention entail the use of a Listeria-based EphA2 vaccine. The invention also provides pharmaceutical compositions comprising one or more Listeria-based vaccines of the invention either alone or in combination with one or more other agents useful for cancer therapy. In certain aspects of the invention, the methods entail eliciting both CD4⁺ and CD8⁺ T-cell responses against EphA2 and/or EphA2-expressing cells.

This application claims priority to U.S. provisional application Ser. No. 60/511,919, filed Oct. 15, 2003, U.S. provisional application Ser. No. 60/511,719, filed Oct. 15, 2003, U.S. provisional application Ser. No. 60/532,666, filed Dec. 24, 2003, U.S. provisional application Ser. No. 60/556,631, filed Mar. 26, 2004, U.S. provisional application Ser. No. ______, filed Oct. 1, 2004 (Attorney Docket No. 10271-144-888), and U.S. provisional application Ser. No. ______, filed Oct. 7, 2004 (Attorney Docket No. 10271-146-888), each of which is incorporated by reference in its entirety.

### 1. FIELD OF THE INVENTION

The present invention relates to methods and compositions for the treatment, management, or prevention of proliferative cell disease. The present invention further relates to Listeria-based compositions for eliciting an immune response against hyperproliferative cells and methods of using the compositions. The invention encompasses, inter alia, vaccines comprising Listeria that express an EphA2 antigenic peptide and the administration of such an EphA2 vaccine for eliciting an immune response against hyperproliferative cells that express EphA2. The invention also provides vaccines comprising one or more Listeria-based compositions of the invention in combination with one or more other agents useful for therapy of proliferative disorders.

### 2. BACKGROUND OF THE INVENTION

### 2.1. Listeria

Listeria monocytogenes (Listeria) is a Gram-positive facultative intracellular bacterium that is being developed for use in antigen-specific vaccines due to its ability to prime a potent CD4+/CD8+ T-cell mediated response via both MHC class I and class II antigen presentation pathways, and as such it has been tested recently as a vaccine vector in a human clinical trial among normal healthy volunteers.

Listeria has been studied for many years as a model for stimulating both innate and adaptive T cell-dependent antibacterial immunity. The ability of Listeria to effectively stimulate cellular immunity is based on its intracellular lifecycle. Upon infecting the host, the bacterium is rapidly taken up by phagocytes including macrophages and dendritic cells into a phagolysosomal compartment. The majority of the bacteria are subsequently degraded. Peptides resulting from proteolytic degradation of pathogens within phagosomes of infected APCs are loaded directly onto MHC class II molecules, and these MHC II-peptide complexes activate CD4+ “helper” T cells that stimulate the production of antibodies, and the processed antigens are expressed on the surface of the antigen presenting cell via the class II endosomal pathway. Within the acidic compartment, certain bacterial genes are activated including the cholesterol-dependent cytolysin, LLO, which can degrade the phagolysosome, releasing the bacterium into the cytosolic compartment of the host cell, where the surviving Listeria propagate. Efficient presentation of heterologous antigens via the MHC class I pathway requires de novo endogenous protein expression by Listeria. Within antigen presenting cells (APC), proteins synthesized and secreted by Listeria are sampled and degraded by the proteosome. The resulting peptides are shuttled into the endoplasmic reticulum by TAP proteins and loaded onto MHC class I molecules. The MHC I-peptide complex is delivered to the cell surface, which in combination with sufficient co-stimulation (signal 2) activates and stimulates cytotoxic T lymphocytes (CTLs) having the cognate T cell receptor to expand and subsequently recognize the MHC I-peptide complex.

### 2.2. Hyperproliferative Diseases

2.2.1. Cancer

A neoplasm, or tumor, is a neoplastic mass resulting from abnormal uncontrolled cell growth which can be benign or malignant. Benign tumors generally remain localized. Malignant tumors are collectively termed cancers. The term “malignant” generally means that the tumor can invade and destroy neighboring body structures and spread to distant sites to cause death (for review, see Robbins and Angell, 1976, Basic Pathology, 2d Ed., W. B. Saunders Co., Philadelphia, pp. 68-122). Cancer can arise in many sites of the body and behaves differently depending upon its origin. Cancerous cells destroy the part of the body in which they originate and then spread to other part(s) of the body where they start new growth and cause more destruction.

More than 1.2 million Americans develop cancer each year. Cancer is the second leading cause of death in the United States and, if current trends continue, cancer is expected to be the leading cause of death by the year 2010. Lung and prostate cancer are the top cancer killers for men in the United States. Lung and breast cancer are the top cancer killers for women in the United States. One in two men in the United States will be diagnosed with cancer at some time during his lifetime. One in three women in the United States will be diagnosed with cancer at some time during her lifetime.

A cure for cancer has yet to be found. Current treatment options, such as surgery, chemotherapy and radiation treatment, are often either ineffective or present serious side effects.

2.2.2. Metastasis

The most life-threatening forms of cancer often arise when a population of tumor cells gains the ability to colonize distant and foreign sites in the body. These metastatic cells survive by overriding restrictions that normally constrain cell colonization into dissimilar tissues. For example, typical mammary epithelial cells will generally not grow or survive if transplanted to the lung, yet lung metastases are a major cause of breast cancer morbidity and mortality. Recent evidence suggests that dissemination of metastatic cells through the body can occur long before clinical presentation of the primary tumor. These micrometastatic cells may remain dormant for many months or years following the detection and removal of the primary tumor. Thus, a better understanding of the mechanisms that allow for the growth and survival of metastatic cells in a foreign microenvironment is critical for the improvement of therapeutics designed to fight metastatic cancer and diagnostics for the early detection and localization of metastases.

2.2.3. Cancer Cell Signaling

Cancer is a disease of aberrant signal transduction. Aberrant cell signaling overrides anchorage-dependent constraints on cell growth and survival (Rhim et al., 1997, Crit. Rev. in Oncogenesis 8:305; Patarca, 1996, Crit. Rev. in Oncogenesis 7:343; Malik et al., 1996, Biochimica et Biophysica Acta 1287:73; Cance et al., 1995, Breast Cancer Res. Treat. 35:105). Tyrosine kinase activity is induced by extracellular matrix (ECM) anchorage and indeed, the expression or function of tyrosine kinases is usually increased in malignant cells (Rhim et al., 1997, Critical Reviews in Oncogenesis 8:305; Cance et al., 1995, Breast Cancer Res. Treat. 35:105; Hunter, 1997, Cell 88:333). Based on evidence that tyrosine kinase activity is necessary for malignant cell growth, tyrosine kinases have been targeted with new therapeutics (Levitzki et al., 1995, Science 267:1782; Kondapaka et al., 1996, Mol. & Cell. Endocrinol. 117:53; Fry et al., 1995, Curr. Opin. in BioTechnology 6:662). Unfortunately, obstacles associated with specific targeting to tumor cells often limit the application of these drugs. In particular, tyrosine kinase activity is often vital for the function and survival of benign tissues (Levitzki et al., 1995, Science 267:1782). To minimize collateral toxicity, it is critical to first identify and then target tyrosine kinases that are selectively overexpressed in tumor cells.

2.2.4. Cancer Therapy

Barriers to the development of anti-metastasis agents have been the assay systems that are used to design and evaluate these drugs. Most conventional cancer therapies target rapidly growing cells. However, cancer cells do not necessarily grow more rapidly but instead survive and grow under conditions that are non-permissive to normal cells (Lawrence and Steeg, 1996, World J. Urol. 14:124-130). These fundamental differences between the behavior of normal and malignant cells provide opportunities for therapeutic targeting. The paradigm that micrometastatic tumors have already disseminated throughout the body emphasizes the need to evaluate potential chemotherapeutic drugs in the context of a foreign and three-dimensional microenvironment. Many standard cancer drug assays measure tumor cell growth or survival under typical cell culture conditions (i.e., monolayer growth). However, cell behavior in two-dimensional assays often does not reliably predict tumor cell behavior in vivo.

Currently, cancer therapy may involve surgery, chemotherapy, hormonal therapy and/or radiation treatment to eradicate neoplastic cells in a patient (see, e.g., Stockdale, 1998, “Principles of Cancer Patient Management,” in Scientific American: Medicine, vol. 3, Rubenstein and Federman, eds., ch. 12, sect. IV). Recently, cancer therapy may also involve biological therapy or immunotherapy. All of these approaches can pose significant drawbacks for the patient. Surgery, for example, may be contraindicated due to the health of the patient or may be unacceptable to the patient. Additionally, surgery may not completely remove the neoplastic tissue. Radiation therapy is only effective when the neoplastic tissue exhibits a higher sensitivity to radiation than normal tissue, and radiation therapy can also often elicit serious side effects. Hormonal therapy is rarely given as a single agent and, although it can be effective, is often used to prevent or delay recurrence of cancer after other treatments have removed the majority of the cancer cells. Biological therapies/immunotherapies are limited in number and each therapy is generally effective for only a very specific type of cancer.

With respect to chemotherapy, there are a variety of chemotherapeutic agents available for treatment of cancer. A significant majority of cancer chemotherapeutics act by inhibiting DNA synthesis, either directly, or indirectly by inhibiting the biosynthesis of the deoxyribonucleotide triphosphate precursors, to prevent DNA replication and concomitant cell division (see, e.g., Gilman et al., 1990, Goodman and Gilman's: The Pharmacological Basis of Therapeutics, 8th Ed. (Pergamom Press, New York)). These agents, which include alkylating agents, such as nitrosourea, anti-metabolites, such as methotrexate and hydroxyurea, and other agents, such as etoposides, campathecins, bleomycin, doxorubicin, daunorubicin, etc., although not necessarily cell cycle specific, kill cells during S phase because of their effect on DNA replication. Other agents, specifically colchicine and the vinca alkaloids, such as vinblastine and vincristine, interfere with microtubule assembly resulting in mitotic arrest. Chemotherapy protocols generally involve administration of a combination of chemotherapeutic agents to increase the efficacy of treatment.

Despite the availability of a variety of chemotherapeutic agents, chemotherapy has many drawbacks (see, e.g., Stockdale, 1998, “Principles Of Cancer Patient Management” in Scientific American Medicine, vol. 3, Rubenstein and Federman, eds., ch. 12, sect. X). Almost all chemotherapeutic agents are toxic, and chemotherapy causes significant, and often dangerous, side effects, including severe nausea, bone marrow depression, immunosuppression, etc. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even those agents that act by mechanisms different from the mechanisms of action of the drugs used in the specific treatment; this phenomenon is termed pleiotropic drug or multidrug resistance. Thus, because of drug resistance, many cancers prove refractory to standard chemotherapeutic treatment protocols.

There is a significant need for alternative cancer treatments, particularly for treatment of cancer that has proved refractory to standard cancer treatments, such as surgery, radiation therapy, chemotherapy, and hormonal therapy. Further, it is uncommon for cancer to be treated by only one method. Thus, there is a need for development of new therapeutic agents for the treatment of cancer and new, more effective, therapy combinations for the treatment of cancer.

2.2.5. Other Hyperproliferative Disorders

2.2.5.1. Asthma

Asthma is a disorder characterized by intermittent airway obstruction. In western countries, it affects 15% of the pediatric population and 7.5% of the adult population (Strachan et al., 1994, Arch. Dis. Child 70:174-178). Most asthma in children and young adults is initiated by IgE mediated allergy (atopy) to inhaled allergens such as house dust mite and cat dander allergens. However, not all asthmatics are atopic, and most atopic individuals do not have asthma. Thus, factors in addition to atopy are necessary to induce the disorder (Fraser et al., eds.,1994, Synopsis of Diseases of the Chest: 635-53 (WB Saunders Company, Philadelphia); Djukanovic et al., 1990, Am. Rev. Respir. Dis. 142:434-457). Asthma is strongly familial, and is due to the interaction between genetic and environmental factors. The genetic factors are thought to be variants of normal genes (“polymorphisms”) which alter their function to predispose to asthma.

Asthma may be identified by recurrent wheeze and intermittent air flow limitation. An asthmatic tendency may be quantified by the measurement of bronchial hyper-responsiveness in which an individual's dose-response curve to a broncho-constrictor such as histamine or methacholine is constructed. The curve is commonly summarized by the dose which results in a 20% fall in air flow (PD20) or the slope of the curve between the initial air flow measurement and the last dose given (slope).

In the atopic response, IgE is produced by B-cells in response to allergen stimulation. These antibodies coat mast cells by binding to the high affinity receptor for IgE and initiate a series of cellular events leading to the destabilization of the cell membrane and release of inflammatory mediators. This results in mucosal inflammation, wheezing, coughing, sneezing and nasal blockage.

Atopy can be diagnosed by (i) a positive skin prick test in response to a common allergen; (ii) detecting the presence of specific serum IgE for allergen; or (iii) by detecting elevation of total serum IgE.

2.2.5.2. COPD

Chronic obstructive pulmonary disease (COPD) is an umbrella term frequently used to describe two conditions of fixed airways disorders, chronic bronchitis and emphysema. Chronic bronchitis and emphysema are most commonly caused by smoking; approximately 90% of patients with COPD are or were smokers. Although approximately 50% of smokers develop chronic bronchitis, only 15% of smokers develop disabling airflow obstruction. Certain animals, particularly horses, suffer from COPD as well.

The airflow obstruction associated with COPD is progressive, may be accompanied by airway hyperactivity, and may be partially reversible. Non-specific airway hyper-responsiveness may also play a role in the development of COPD and may be predictive of an accelerated rate of decline in lung function.

COPD is a significant cause of death and disability. It is currently the fourth leading cause of death in the United States and Europe. Treatment guidelines advocate early detection and implementation of smoking cessation programs to help reduce morbidity and mortality due to the disorder. However, early detection and diagnosis has been difficult for a number of reasons. COPD takes years to develop and acute episodes of bronchitis often are not recognized by the general practitioner as early signs of COPD. Many patients exhibit features of more than one disorder (e.g., chronic bronchitis or asthmatic bronchitis) making precise diagnosis a challenge, particularly early in the etiology of the disorder. Also, many patients do not seek medical help until they are experiencing more severe symptoms associated with reduced lung function, such as dyspnea, persistent cough, and sputum production. As a consequence, the vast majority of patients are not diagnosed or treated until they are in a more advanced stage of the disorder.

2.2.5.3. Mucin

Mucins are a family of glycoproteins secreted by the epithelial cells including those at the respiratory, gastrointestinal and female reproductive tracts. Mucins are responsible for the viscoelastic properties of mucus (Thornton et al., 1997, J. Biol. Chem. 272:9561-9566). Nine mucin genes are known to be expressed in man: MUC 1, MUC 2, MUC 3, MUC 4, MUC 5AC, MUC 5B, MUC 6, MUC 7 and MUC 8 (Bobek et al., 1993, J. Biol. Chem. 268:20563-9; Dusseyn et al., 1997, J. Biol. Chem. 272:3168-78; Gendler et al., 1991, Am. Rev.Resp. Dis. 144:S42-S47; Gum et al., 1989, J. Biol. Chem. 264:6480-6487; Gum et al., 1990, Biochem. Biophys. Res. Comm. 171:407-415; Lesuffleur et al., 1995, J. Biol. Chem. 270:13665-13673; Meerzaman et al., 1994, J. Biol. Chem. 269:12932-12939; Porchet et al., 1991, Biochem. Biophys. Res. Comm. 175:414-422; Shankar et al., 1994, Biochem. J. 300:295-298; Toribara et al., 1997, J. Biol. Chem. 272:16398-403). Many airway disorders such chronic bronchitis, chronic obstructive pulmonary disease, bronchietactis, asthma, cystic fibrosis and bacterial infections are characterized by mucin overproduction (Prescott et al., Eur. Respir. J., 1995, 8:1333-1338; Kim et al., Eur. Respir. J., 1997, 10:1438; Steiger et al., 1995, Am. J Respir. Cell Mol. Biol., 12:307-314). Mucociliary impairment caused by mucin hypersecretion leads to airway mucus plugging which promotes chronic infection, airflow obstruction and sometimes death. For example, COPD, a disorder characterized by slowly progressive and irreversible airflow limitation, is a major cause of death in developed countries. The respiratory degradation consists mainly of decreased luminal diameters due to airway wall thickening and increased mucus caused by goblet cell hyperplasia and hypersecretion. Epidermal growth factor (EGF) is known to upregulate epithelial cell proliferation, and mucin production/secretion (Takeyama et al., 1999, Proc. Natl. Acad. Sci. USA 96:3081-6; Burgel et al., 2001, J. Immunol. 167:5948-54). EGF also causes mucin-secreting cells, such as goblet cells, to proliferate and increase mucin production in airway epithelia (Lee et al., 2000, Am. J. Physiol. Lung Cell. Mol. Physiol. 278:185-92; Takeyama et al., 2001, Am. J. Respir. Crit. Care. Med. 163:511-6; Burgel et al., 2000, J. Allergy Clin. Immunol. 106:705-12). Historically, mucus hypersecretion has been treated in two ways: physical methods to increase clearance and mucolytic agents. Neither approach has yielded significant benefit to the patient or reduced mucus obstruction. Therefore, it would be desirable to have methods for reducing mucin production and treating the disorders associated with mucin hypersecretion.

2.2.5.4. Restenosis

Vascular interventions, including angioplasty, stenting, atherectomy and grafting are often complicated by undesirable effects. Exposure to a medical device which is implanted or inserted into the body of a patient can cause the body tissue to exhibit adverse physiological reactions. For instance, the insertion or implantation of certain catheters or stents can lead to the formation of emboli or clots in blood vessels. Other adverse reactions to vascular intervention include endothelial cell proliferation which can lead to hyperplasia, restenosis, i.e. the re-occlusion of the artery, occlusion of blood vessels, platelet aggregation, and calcification. Treatment of restenosis often involves a second angioplasty or bypass surgery. In particular, restenosis may be due to endothelial cell injury caused by the vascular intervention in treating a restenosis.

Angioplasty involves insertion of a balloon catheter into an artery at the site of a partially obstructive atherosclerotic lesion. Inflation of the balloon is intended to rupture the intima and dilate the obstruction. About 20 to 30% of obstructions reocclude in just a few days or weeks (Eltchaninoff et al., 1998, J. Am Coll. Cardiol. 32:980-984). Use of stents reduces the re-occlusion rate, however a significant percentage continues to result in restenosis. The rate of restenosis after angioplasty is dependent upon a number of factors including the length of the plaque. Stenosis rates vary from 10% to 35% depending the risk factors present. Further, repeat angiography one year later reveals an apparently normal lumen in only about 30% of vessels having undergone the procedure.

Restenosis is caused by an accumulation of extracellular matrix containing collagen and proteoglycans in association with smooth muscle cells which is found in both the atheroma and the arterial hyperplastic lesion after balloon injury or clinical angioplasty. Some of the delay in luminal narrowing with respect to smooth muscle cell proliferation may result from the continuing elaboration of matrix materials by neointimal smooth muscle cells. Various mediators may alter matrix synthesis by smooth muscle cells in vivo.

2.2.5.5. Neointimal Hyperplasia

Neointimal hyperplasia is the pathological process that underlies graft atherosclerosis, stenosis, and the majority of vascular graft occlusion. Neointimal hyperplasia is commonly seen after various forms of vascular injury and a major component of the vein graft's response to harvest and surgical implantation into high-pressure arterial circulation.

Smooth muscle cells in the middle layer (i.e. media layer) of the vessel wall become activated, divide, proliferate and migrate into the inner layer (i.e. intima layer). The resulting abnormal neointimal cells express pro-inflammatory molecules, including cytokines, chemokines and adhesion molecules that further trigger a cascade of events that lead to occlusive neointimal disease and eventually graft failure.

The proliferation of smooth muscle cells is a critical event in the neointimal hyperplastic response. Using a variety of approaches, studies have clearly demonstrated that blockade of smooth muscle cell proliferation resulted in preservation of normal vessel phenotype and function, causing the reduction of neointimal hyperplasia and graft failure.

Existing treatments for the indications discussed above is inadequate; thus, there exists a need for improved treatments for the above indications.

### 2.3. EphA2

EphA2 is a 130 kDa receptor tyrosine kinase that is expressed in adult epithelia, where it is found at low levels and is enriched within sites of cell-cell adhesion (Zantek et al, 1999, Cell Growth & Differentiation 10:629; Lindberg et al., 1990, Molecular & Cellular Biology 10:6316). This subcellular localization is important because EphA2 binds ligands (known as EphrinsA1 to A5) that are anchored to the cell membrane (Eph Nomenclature Committee, 1997, Cell 90:403; Gale et al., 1997, Cell & Tissue Research 290: 227). The primary consequence of ligand binding is EphA2 autophosphorylation (Lindberg et al., 1990, supra). However, unlike other receptor tyrosine kinases, EphA2 retains enzymatic activity in the absence of ligand binding or phosphotyrosine content (Zantek et al., 1999, supra). EphA2 is upregulated on a large number hyperproliferating cells, including aggressive carcinoma cells.

### 3. SUMMARY OF THE INVENTION

EphA2 is overexpressed and functionally altered in a large number of malignant carcinomas. EphA2 is an oncoprotein and is sufficient to confer metastatic potential to cancer cells. EphA2 is also associated with other hyperproliferating cells and is implicated in diseases caused by cell hyperproliferation. The present invention stems from the inventors' discovery that administration of Listeria that express an EphA2 antigenic peptide to a subject provides beneficial therapeutic and prophylactic benefits against hyperproliferative disorders involving EphA2 overexpressing cells. Without being bound by any mechanism or theory, it is believed that the therapeutic and prophylactic benefit is the result of an immune response elicited by administration of the EphA2 antigenic peptide-expressing Listeria.

The present invention thus provides Listeria-based EphA2 vaccines and methods for their use. The Listeria-based EphA2 vaccines of the present invention can elicit a cellular immune response, a humoral immune response, or both. Where the immune response is a cellular immune response, it can be a Tc, Th1 or a Th2 immune response. In a preferred embodiment, the immune response is a Th2 cellular immune response.

In a preferred embodiment, a Listeria-based EphA2 vaccine of the invention expresses one or more epitopes of EphA2 that is selectively exposed or increased on cancer cells relative to non-cancer cells (i.e., normal, healthy cells or cells that are not hyperproliferative). In one embodiment, the cancer is of an epithelial cell origin. In other embodiments, the cancer is a cancer of the skin, lung, colon, prostate, breast, ovary, esophageal, bladder, or pancreas or is a renal cell carcinoma or a melanoma. In another embodiment, the cancer is of a T cell origin. In yet other embodiments, the cancer is a leukemia or a lymphoma.

In a preferred embodiment, the methods and compositions of the invention are used to prevent, treat or manage EphA2-expressing tumor metastases. In a preferred embodiment, the EphA2-expressing cells against which an immune response is sought (“target cells”) overexpress EphA2 relative to a normal healthy cell of the same type as assessed by an assay described herein or known to one of skill in the art (e.g., an immunoassay such as an ELISA or a Western blot, a Northern blot or RT-PCR). In a preferred embodiment, less EphA2 on the target cells is bound to ligand compared to a normal, healthy cell of the same type, either as a result of decreased cell-cell contacts, altered subcellular localization, or increases in amount of EphA2 relative to ligand. In another embodiment, approximately 10% or less, approximately 15% or less, approximately 20% or less, approximately 25% or less, approximately 30% or less, approximately 35% or less, approximately 40% or less, approximately 45% or less, approximately 50% or less, approximately 55% or less, approximately 60% or less, approximately 65% or less, approximately 70% or less, approximately 75% or less, approximately 80% or less, approximately 85% or less, approximately 90% or less, or approximately 95% or less of EphA2 on the target cells is bound to ligand (e.g., EphrinA 1) compared to a normal, healthy cell of the same type as assessed by an assay known in the art (e.g., an immunoassay). In another embodiment, 1-10 fold, 1-8 fold, 1-5 fold, 1-4 fold or 1-2 fold, or 1 fold, 1.5 fold, 2 fold, 3 fold, 4 fold, 5 fold, or 10 fold less EphA2 on target cells is bound to ligand (e.g., EphrinA1) compared to a normal, healthy cell of the same type as assessed by an assay known in the art (e.g., an immunoassay).

Thus, the present invention provides methods of eliciting an immune response against an EphA2-expressing cell, said method comprising administering to an individual a Listeria-based EphA2 vaccine in an amount effective to elicit an immune response against an EphA2-expressing cell.

The present invention provides a method of treating, preventing or managing a hyperproliferative disorder of EphA2-expressing cells, said method comprising administering to an individual a Listeria-based EphA2 vaccine in an amount effective treat or prevent the hyperproliferative disorder (e.g., a neoplastic hyperproliferative disorder and a non-neoplastic hyperproliferative disorder). The present invention also provides Listeria-based EphA2 vaccines useful for eliciting an immune response against an EphA2-expressing cell and/or for treating, preventing or managing a hyperproliferative disorder of EphA2-expressing cells.

The Listeria-based EphA2 vaccines may comprise Listeria as an EphA2 antigenic peptide expression vehicle. In a preferred embodiment, the Listeria bacteria administered to a subject (preferably, a human subject) as an EphA2 antigenic expression vehicle are attenuated. For example, the attenuated Listeria bacteria administered to a subject (preferably, a human subject) maybe attenuated in their tissue tropism (e.g., inlB mutant) or ability to spread from cell to cell (e.g., actA mutant). In a specific embodiment, the attenuated Listeria bacteria administered to a subject (preferably, a human subject) as an EphA2 antigenic expression vehicle comprise a mutation (e.g., a deletion, addition or substitution) in one or more intemalins (e.g., inlA and/or inlB) and such mutation results in or contributes to the attenuation of the Listeria. In another embodiment, the attenuated Listeria bacteria administered to a subject (preferably, a human subject) as an EphA2 antigenic expression vehicle are attenuated in their tissue tropism (e.g., inlB mutant) and in their ability to spread from cell to cell (e.g., actA mutant). In a preferred embodiment, the attenuated Listeria bacteria administered to subject (preferably, a human subject) as an EphA2 antigenic expression vehicle comprise a mutation (e.g., a deletion, addition or substitution) in internalin B and a mutation in actA, and such mutations result in or contribute to the attenuation of the Listeria.

The Listeria (preferably, the attenuated Listeria) of the invention are preferably engineered to express an EphA2 antigenic peptide that is secreted from the Listeria. In a specific embodiment, a nucleic acid encoding an EphA2 antigenic peptide comprises a nucleotide sequence encoding a secretory signal, e.g., the SecA secretory signal or Tat signal, operatively linked to the nucleotide sequence encoding the EphA2 antigenic peptide. In some embodiments, the signal sequence is a Listeria signal sequence. In other embodiments, the signal sequence is a bacterial signal sequence other than a Listeria signal sequence (i.e., a non-Listeria bacterial signal sequence).

Strains of Listeria bacteria suitable for use in the methods and compositions of the invention include, but are not limited to, Listeria grayi, Listeria innocua, Listeria ivanovii, Listeria monocytogenes, Listeria seeligeri and Listeria welshimeri. A preferred strain of Listeria bacteria for use in the methods and compositions of the invention is Listeria monocytogenes.

The compositions and methods of the present invention are useful in the treatment, prevention and/or management of hyperproliferative diseases. In certain embodiments, the hyperproliferative disease is cancer. In certain embodiments, the cancer is of an epithelial cell origin and/or involves cells that overexpress EphA2 relative to non-cancer cells having the tissue type of said cancer cells. In specific embodiments, the cancer is a cancer of the skin, lung, colon, breast, ovary, esophageal, prostate, bladder or pancreas or is a renal cell carcinoma or melanoma. In yet other embodiments, the cancer is of a T cell origin. In specific embodiments, the cancer is a leukemia or a lymphoma. In yet other embodiments, the hyperproliferative disorder is non-neoplastic. In specific embodiments, the non-neoplastic hyperproliferative disorder is an epithelial cell disorder. Exemplary non-neoplastic hyperproliferative disorders are asthma, chronic pulmonary obstructive disease, lung fibrosis, bronchial hyper responsiveness, psoriasis, and seborrheic dermatitis. In certain embodiments, the hyperproliferative disease is an endothelial cell disorder.

The EphA2 antigenic peptide for use in accordance with the methods and compositions of the present invention may comprise full length EphA2 or an antigenic fragment, analog or derivative thereof. In certain embodiments, the EphA2 antigenic peptide comprises the extracellular domain of EphA2 or the intracellular domain of EphA2. In certain embodiments, the EphA2 antigenic peptide lacks the EphA2 transmembrane domain. In certain embodiments, the EphA2 antigenic peptide comprises the EphA2 extracellular and intracellular domains and lacks the transmembrane domain of EphA2. In certain embodiments, the EphA2 antigenic peptide comprises full length EphA2 or a fragment thereof with a substitution of lysine to methionine at amino acid residue 646 of EphA2. In certain embodiments, the EphA2 antigenic peptide comprises the extracellular and intracellular domains of EphA2, lacks the transmembrane domain of EphA2 and has a substitution of lysine to methionine at amino acid residue 646 of EphA2. In certain embodiments the EphA2 antigenic peptide is a chimeric polypeptide comprising at least an antigenic portion of EphA2 and a second polypeptide.

An EphA2 antigenic peptide-expressing Listeria may express one or a plurality of EphA2 antigenic peptides. In a specific embodiment, an EphA2 antigenic peptide-expressing Listeria expresses 2, 3, 4, 5, 6, 7, 8, 9, 10, 15, 20, 25 or more EphA2 antigenic peptides, or 2-5, 2-10, 2-20, 10-20, or 15-25 EphA2 antigenic peptides. The plurality of EphA2 antigenic peptides may be expressed from a single expression construct or a plurality of expression constructs. The expression construct(s) can be episomal or integrated into the Listeria genome. For example, in certain embodiments, the genome of the Listeria vaccine strain comprises one or more gene expression cassettes, which in combination encode both the intracellular and extracellular domains of EphA2. In specific embodiments, the one or more expression cassettes are integrated into the Listeria genome.

A vaccine of the invention may have one or a plurality of EphA2 antigenic peptide-expressing Listeria. In a specific embodiment, a vaccine of the invention has 2, 3, 4, 5, 6, 7, 8, 9, 10, 15, 20, 25 or more EphA2 antigenic peptide-expressing Listeria, or 2-5, 2-10, 2-20, 10-20, or 15-25 EphA2 antigenic peptide-expressing Listeria.

The methods of the present invention encompass combination therapy with a Listeria-based EphA2 vaccine and one or more additional therapies, for example an additional anti-cancer therapy. In certain embodiments, the additional anti-cancer therapy is an agonistic EphA2 antibody, i.e., antibody that binds to EphA2 and induces signaling and phosphorylation of EphA2. In other embodiments, the additional anti-cancer therapy is an anti-idiotype of an anti-EphA2 antibody. In yet other embodiments, the additional anti-cancer therapy is chemotherapy, biological therapy, immunotherapy, radiation therapy, hormonal therapy, or surgery.

In certain aspects of the present invention, the Listeria-based vaccines of the invention are administered in combination with a therapy that increases EphA2 internalization. In specific embodiments, the agent is an EphA2 agonist, for example an antibody, peptide (see, e.g., Koolpe et al., 2002, J. Biol. Chem. 277(49):46974-46979) or small molecule. In other specific embodiments, the agent is an inhibitor of a phosphatase that modulates EphA2, e.g., low molecular weight tyrosine phosphatase (LMW-PTP).

The vaccines of the invention can be administered, for example, by mucosal, intranasal, parenteral, intramuscular, intravenous, oral or intraperitoneal routes. In a specific embodiment, the vaccines of the invention are administered locally to the site of a disease, by, e.g., implantation or intratumoral injection.

In other embodiments, the Listeria-based EphA2 vaccines of the invention are used to treat, prevent and/or manage a non-cancer disease or disorder associated with cell hyperproliferation, such as but not limited to asthma, chronic obstructive pulmonary disease, restenosis (smooth muscle and/or endothelial), psoriasis, etc. In preferred embodiments, the hyperproliferative cells are epithelial. In preferred embodiments, the hyperproliferative cells overexpress EphA2. In another preferred embodiment, some (e.g., 5% or less, 10% or less, 15% or less, 20% or less, 25% or less, 30% or less, 35% or less, 40% or less, 45% or less, 50% or less, 55% or less, 60% or less, 75% or less, 85% or less) EphA2 is not bound to ligand as assessed by an assay known in the art (e.g., an immunoassay), either as a result of decreased cell-cell contacts, altered subcellular localization, or increases in the amount of EphA2 relative to EphA2-ligand.

In yet other aspects of the invention, the Listeria-based EphA2 vaccines are used to treat, prevent and/or manage a disorder associated with or involving aberrant angiogenesis. The Listeria-based EphA2 vaccines are used to elicit an immune response against EphA2 expressed on neovasculature. Thus, the present invention provides methods of treating, preventing and/or managing a disorder associated with or involving aberrant angiogenesis comprising administering to a subject in need thereof a composition comprising an EphA2 antigenic peptide-expressing Listeria bacterium in an amount effective to treat, prevent and/or manage a disorder associated with or involving aberrant angiogenesis. Examples of such diseases include, but are not limited to, macular degeneration, diabetic retinopathy, retinopathy of prematurity, vascular restenosis, infantile hemangioma, verruca vulgaris, psoriasis, Kaposi's sarcoma, neurofibromatosis, recessive dystrophic epidermolysis bullosa, rheumatoid arthritis, ankylosing spondylitis, systemic lupus, psoriatic arthropathy, Reiter's syndrome, and Sjogren's syndrome, endometriosis, preeclampsia, atherosclerosis and coronary artery disease.

The methods and compositions of the invention are useful not only in untreated patients but are also useful in the treatment of patients partially or completely refractory to current standard and experimental cancer therapies, including but not limited to chemotherapies, hormonal therapies, biological therapies, radiation therapies, and/or surgery as well as to improve the efficacy of such treatments. In particular, EphA2 expression has been implicated in increasing levels of the cytokine IL-6, which has been associated with the development of cancer cell resistance to different treatment regimens, such as chemotherapy and hormonal therapy. In addition, EphA2 overexpression can override the need for estrogen receptor activity thus contributing to tamoxifen resistance in breast cancer cells. Accordingly, in a preferred embodiment, the invention provides therapeutic and prophylactic methods for the treatment, prevention or management of cancer that has been shown to be or may be refractory or non-responsive to therapies other than those comprising administration of Listeria-based EphA2 vaccines of the invention. In a specific embodiment, one or more Listeria-based EphA2 vaccines of the invention are administered to a patient refractory or non-responsive to a non-EphA2-based treatment, particularly tamoxifen treatment or a treatment in which resistance is associated with increased IL-6 levels, to render the patient non-refractory or responsive. The treatment to which the patient had previously been refractory or non-responsive can then be administered with therapeutic effect.

The methods and compositions of the invention are useful not only in untreated patients but are also useful in the treatment of patients partially or completely refractory to current standard and experimental therapies for non-neoplastic hyperproliferative disorders and/or disorders associated with or involving aberrant angiogenesis. The methods and compositions of the invention are useful for the treatment of patients partially or completely refractory to current standard and experimental therapies for neoplastic hyperproliferative disorders and/or disorders associated with or involving aberrant angiogenesis (e.g., macular degeneration, diabetic retinopathy, retinopathy of prematurity, vascular restenosis, infantile hemangioma, verruca vulgaris, psoriasis, Kaposi's sarcoma, neurofibromatosis, recessive dystrophic epidermolysis bullosa, rheumatoid arthritis, ankylosing spondylitis, systemic lupus, psoriatic arthropathy, Reiter's syndrome, and Sjogren's syndrome, endometriosis, preeclampsia, atherosclerosis and coronary artery disease), asthma, chronic pulmonary obstructive disease, lung fibrosis, bronchial hyper responsiveness, psoriasis, and seborrheic dermatitis).

The present invention also provides kits comprising the vaccines or vaccine components of the invention.

### 3.1. Definitions

As used herein, the term “Listeria-based EphA2 vaccine” refers to a Liseria bacterium that has been engineered to express an EphA2 antigenic peptide, or a composition comprising such a bacterium. The Listeria-based EphA2 vaccines of the invention, when administered in an effective amount, elicit an immune response against EphA2 on hyperproliferative cells. Strains of Listeria bacteria suitable for use in a vaccine of the invention include, but are not limited to, Listeria grayi, Listeria innocua, Listeria ivanovii, Listeria monocytogenes, Listeria seeligeri and Listeria welshimeri. In a preferred embodiment, the Listeria is Listeria monocytogenes.

As used herein, the terms “EphA2 antigenic peptide” and “EphA2 antigenic polypeptide” refer to an EphA2 polypeptide, preferably of SEQ ID NO:2, or a fragment, analog or derivative thereof comprising one or more B cell epitopes or T cell epitopes of EphA2. The EphA2 polypeptide may be from any species. In certain embodiments, an EphA2 polypeptide refers to the mature, processed form of EphA2. In other embodiments, an EphA2 polypeptide refers to an immature form of EphA2.

The nucleotide and/or amino acid sequences of EphA2 polypeptides can be found in the literature or public databases, or the nucleotide and/or amino acid sequences can be determined using cloning and sequencing techniques known to one of skill in the art. For example, the nucleotide sequence of human EphA2 can be found in the GenBank database (see, e.g., Accession Nos. BC037166, M59371 and M36395). The amino acid sequence of human EphA2 can be found in the GenBank database (see, e.g., Accession Nos. NP_{—}004422, AAH37166 and AAA53375). Additional non-limiting examples of amino acid sequences of EphA2 are listed in Table 1, infra.

TABLE 1

| Species | GenBank Accession No. |
|---|---|
| Mouse | NP_034269, AAH06954 |
| Rat | XP_345597 |
| Chicken | BAB63910 |

In certain embodiments, the EphA2 antigenic peptides are not one or more of the following peptides: TLADFDPRV (SEQ ID NO:3); VLLLVLAGV (SEQ ID NO:4); VLAGVGFFI (SEQ ID NO:5); IMNDMPIYM (SEQ ID NO:6); SLLGLKDQV (SEQ ID NO:7); WLVPIGQCL (SEQ ID NO:8); LLWGCALAA (SEQ ID NO:9); GLTRTSVTV (SEQ ID NO:10); NLYYAESDL (SEQ ID NO:11); KLNVEERSV (SEQ ID NO:12); IMGQFSHHN (SEQ ID NO:13); YSVCNVMSG (SEQ ID NO:14); MQNIMNDMP (SEQ ID NO:15); EAGIMGQFSHHNIIR (SEQ ID NO:16); PIYMYSVCNVMSG (SEQ ID NO:17); DLMQNIMNDMPIYMYS (SEQ ID NO:18). In certain specific embodiments, the EphA2 antigenic peptide is not any of SEQ ID NO:3-12, is not SEQ ID NO:13-15, and/or is not SEQ ID NO:16-18. In yet another specific enbodiment, the EphA2 antigenic peptide is not SEQ ID NO:3-18.

As used herein, the term “analog” in the context of a proteinaceous agent (e.g., a peptide, polypeptide, protein or antibody) refers to a proteinaceous agent that possesses a similar or identical function as a second proteinaceous agent (e.g., an EphA2 polypeptide) but does not necessarily comprise a similar or identical amino acid sequence or structure of the second proteinaceous agent. A proteinaceous agent that has a similar amino acid sequence refers to a proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 20 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure of the second proteinaceous agent. The structure of a proteinaceous agent can be determined by methods known to those skilled in the art, including but not limited to, X-ray crystallography, nuclear magnetic resonance, and crystallographic electron microscopy. Preferably, the proteinaceous agent has EphA2 activity.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87: 2264-2268, modified as in Karlin and Altschul, 1993 , Proc. Natl. Acad. Sci. U.S.A. 90: 5873-5877. Such an algorithm is incorporated into the NBLAST and XBLAST programs of Altschul et al., 1990, J. Mol. Biol. 215: 403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25: 3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4: 11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possesses a similar or identical function as a first organic or inorganic molecule and is structurally similar to the first organic or inorganic molecule.

As used herein, the terms “attenuated” and “attenuation” refer to a modification(s) so that the Listeria are less pathogenic. The end result of attenuation is that the risk of toxicity as well as other side effects is decreased when the Listeria are administered to a subject.

As used herein, the term “derivative” in the context of a proteinaceous agent (e.g., proteins, polypeptides, peptides, and antibodies) refers to a proteinaceous agent that comprises the amino acid sequence which has been altered by the introduction of amino acid residue substitutions, deletions, and/or additions. The term “derivative” as used herein also refers to a proteinaceous agent which has been modified, i.e., by the covalent attachment of a type of molecule to the proteinaceous agent. For example, but not by way of limitation, a derivative of a proteinaceous agent may be produced, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may also be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses an identical function(s) as the proteinaceous agent from which it was derived.

As used herein, the term “derivative” in the context of EphA2 proteinaceous agents refers to a proteinaceous agent that comprises an amino acid sequence of an EphA2 polypeptide or a fragment of an EphA2 polypeptide that has been altered by the introduction of amino acid residue substitutions, deletions or additions (i.e., mutations). The term “derivative” as used herein in the context of EphA2 proteinaceous agents also refers to an EphA2 polypeptide or a fragment of an EphA2 polypeptide which has been modified, i.e, by the covalent attachment of any type of molecule to the polypeptide. For example, but not by way of limitation, an EphA2 polypeptide or a fragment of an EphA2 polypeptide may be modified, e.g., by glycosylation, acetylation, pegylation, phosphorylation, amidation, derivatization by known protecting/blocking groups, proteolytic cleavage, linkage to a cellular ligand or other protein, etc. A derivative of an EphA2 polypeptide or a fragment of an EphA2 polypeptide may be modified by chemical modifications using techniques known to those of skill in the art, including, but not limited to, specific chemical cleavage, acetylation, formylation, metabolic synthesis of tunicamycin, etc. Further, a derivative of an EphA2 polypeptide or a fragment of an EphA2 polypeptide may contain one or more non-classical amino acids. In one embodiment, a polypeptide derivative possesses a similar or identical function as an EphA2 polypeptide or a fragment of an EphA2 polypeptide described herein. In another embodiment, a derivative of EphA2 polypeptide or a fragment of an EphA2 polypeptide has an altered activity when compared to an unaltered polypeptide. For example, a derivative of an EphA2 polypeptide or fragment thereof can differ in phosphorylation relative to an EphA2 polypeptide or fragment thereof.

As used herein, the term “derivative” in the context of a non-proteinaceous agent refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl, nitryl, or amine group. An organic molecule may also, for example, be esterified, alkylated and/or phosphorylated.

As used herein, the term “EphrinA1 polypeptide” refers to EphrinA1, an analog, derivative or a fragment thereof, or a fusion protein comprising EphrinA1, an analog, derivative or a fragment thereof. The EphrinA1 polypeptide may be from any species. In certain embodiments, the term “EphrinA1 polypeptide” refers to the mature, processed form of EphrinA1. In other embodiments, the term “EphrinA1 polypeptide” refers to an immature form of EphrinA1. In accordance with this embodiment, the antibodies of the invention immunospecifically bind to the portion of the immature form of EphrinA1 that corresponds to the mature, processed form of EphrinA1.

The nucleotide and/or amino acid sequences of EphrinA1 polypeptides can be found in the literature or public databases, or the nucleotide and/or amino acid sequences can be determined using cloning and sequencing techniques known to one of skill in the art. For example, the nucleotide sequence of human EphrinA1 can be found in the GenBank database (see, e.g., Accession No. BC032698). The amino acid sequence of human EphrinA1 can be found in the GenBank database (see, e.g., Accession No. AAH32698). Additional non-limiting examples of amino acid sequences of EphrinA1 are listed in Table 2, infra.

TABLE 2

| Species | GenBank Accession No. |
|---|---|
| Mouse | NP_034237 |
| Rat | NP_446051 |

In a specific embodiment, a EphrinA1 polypeptide is EphrinA1 from any species. In a preferred embodiment, an EphrinA1 polypeptide is human EphrinA1.

As used herein, the term “effective amount” refers to the amount of a therapy (e.g., a prophylactic or therapeutic agent) which is sufficient to reduce and/or ameliorate the severity and/or duration of a disorder (e.g., cancer, a non-neoplastic hyperproliferative cell disorder or a disorder associated with aberrant angiogenesis) or a symptom thereof, prevent the advancement of said disorder, cause regression of said disorder, prevent the recurrence, development, or onset of one or more symptoms associated with said disorder, or enhance or improve the prophylactic or therapeutic effect(s) of another therapy (e.g., prophylactic or therapeutic agent).

As used herein, the term “B cell epitope” refers to a portion of an EphA2 polypeptide having antigenic or immunogenic activity in an animal, preferably in a mammal, and most preferably in a mouse or a human. An epitope having immunogenic activity is a portion of an EphA2 polypeptide that elicits an antibody response in an animal. An epitope having antigenic activity is a portion of an EphA2 polypeptide to which an antibody immunospecifically binds as determined by any method well known in the art, for example, by immunoassays. Antigenic epitopes need not necessarily be immunogenic.

As used herein, the term “T cell epitope” refers to at least a portion of an EphA2 polypeptide, preferably an EphA2 polypeptide of SEQ ID NO:2, that is recognized by a T cell receptor. The term “T cell epitope” encompasses helper T cell (Th) epitopes and cytotoxic T cell (Tc) epitopes. The term “helper T cell epitopes” encompasses Th1 and Th2 epitopes.

As used herein, the term “fragments” in the context of EphA2 polypeptides include an EphA2 antigenic peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of an EphA2 polypeptide.

As used herein, the term “fusion protein” refers to a polypeptide or protein that comprises the amino acid sequence of a first polypeptide or protein or fragment, analog or derivative thereof, and the amino acid sequence of a heterologous polypeptide or protein. In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides, or peptides with immunomodulatory activity may be fused together to form a fusion protein. In a preferred embodiment, fusion proteins retain or have improved activity relative to the activity of the original polypeptide or protein prior to being fused to a heterologous protein, polypeptide, or peptide.

As used herein, the term “heterologous,” in the context of a nucleic acid sequence (e.g., a gene) or an amino acid sequence (e.g., a peptide, polypeptide or protein) refers a nucleic acid sequence or an amino acid sequence that is not found in nature to be associated with a second nucleic acid sequence or a second amino acid sequence (e.g., a nucleic acid sequence or an amino acid sequence derived from a different species).

As used herein, the terms “hyperproliferative cell disorder,” “hyperproliferative cell disease,” “hyperproliferative disorder,” and “hyperproliferative disease” and analogous terms refer to a disorder in which cellular hyperproliferation or any form of excessive cell accumulation causes or contributes to the pathological state or symptoms of the disorder. In some embodiments, the hyperproliferative cell disorder is characterized by hyperproliferating epithelial cells. In other embodiments, the hyperproliferative cell disorder is characterized by hyperproliferating endothelial cells. In other embodiments, the hyperproliferative cell disorder is characterized by hyperproliferating fibroblasts. In certain embodiments, the hyperproliferative cell disorder is not neoplastic. Exemplary non-neoplastic hyperproliferative cell disorders are asthma, chronic pulmonary obstructive disease, fibrosis (e.g., lung, liver, and kidney fibrosis), bronchial hyper responsiveness, psoriasis, and seborrheic dermatitis. In a preferred embodiment, the hyperproliferative cell disorder is characterized by hyperproliferating cells that express (preferably, overexpress) EphA2.

As used herein, the term “immunospecifically binds to EphA2” and analogous terms refers to peptides, polypeptides, proteins, fusion proteins, and antibodies or fragments thereof that specifically bind to an EphA2 receptor or one or more fragments thereof and do not specifically bind to other receptors or fragments thereof. The terms “immunospecifically binds to EphrinA1” and analogous terms refer to peptides, polypeptides, proteins, fusion proteins, and antibodies or fragments thereof that specifically bind to EphrinA1 or one or more fragments thereof and do not specifically bind to other ligands or fragments thereof. A peptide, polypeptide, protein, or antibody that immunospecifically binds to EphA2 or EphrinA1, or fragments thereof, may bind to other peptides, polypeptides, or proteins with lower affinity as determined by, e.g., immunoassays or other assays known in the art to detect binding affinity. Antibodies or fragments that immunospecifically bind to EphA2 or EphrinA1 may be cross-reactive with related antigens. Preferably, antibodies or fragments thereof that immunospecifically bind to EphA2 or EphrinA1 can be identified, for example, by immunoassays or other techniques known to those of skill in the art. An antibody or fragment thereof binds specifically to EphA2 or EphrinA1 when it binds to EphA2 or EphrinA1 with higher affinity than to any cross-reactive antigen as determined using experimental techniques, such as radioimmunoassays (RIAs) and enzyme-linked immunosorbent assays (ELISAs). See, e.g., Paul, ed., 1989, Fundamental Immunology, 2^{nd} ed., Raven Press, New York at pages 332-336 for a discussion regarding antibody specificity. In a preferred embodiment, an antibody that immunospecifically binds to EphA2 or EphrinA1 does not bind or cross-react with other antigens. In another embodiment, an antibody that binds to EphA2 or EphrinA1 that is a fusion protein specifically binds to the portion of the fusion protein that is EphA2 or EphrinA1.

Antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific and bi-specific, etc.), Fab fragments, F(ab′) fragments, disulfide-linked Fvs (sdFv), anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen-binding site that immunospecifically binds to an EphA2 antigen or an EphrinA1 antigen (e.g., one or more complementarity determining regions (CDRs) of an anti-EphA2 antibody or of an anti-EphrinA1 antibody). The antibodies of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

As used herein, the term “isolated” in the context of an organic or inorganic molecule (whether it be a small or large molecule), other than a proteinaceous agent or a nucleic acid, refers to an organic or inorganic molecule substantially free of a different organic or inorganic molecule. Preferably, an organic or inorganic molecule is 60%, 65%, 70%, 75%, 80%, 85%, 90%, 95%, or 99% free of a second, different organic or inorganic molecule. In a preferred embodiment, an organic and/or inorganic molecule is isolated.

As used herein, the term “isolated” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, peptide, or antibody (also referred to as a “contaminating protein”). When the proteinaceous agent is recombinantly produced, it is also preferably substantially free of culture medium, i.e., culture medium represents less than about 20%, 10%, or 5% of the volume of the proteinaceous agent preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. In a specific embodiment, proteinaceous agents disclosed herein are isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, is preferably substantially free of other cellular material, or culture medium when produced by recombinant techniques, or substantially free of chemical precursors or other chemicals when chemically synthesized. In a specific embodiment, nucleic acid molecules are isolated.

As used herein, the term “disease” and “disorder” are used interchangeably to refer to a condition.

As used herein, the term “in combination” refers to the use of more than one therapies (e.g., prophylactic and/or therapeutic agents). The use of the term “in combination” does not restrict the order in which therapies (e.g., prophylactic and/or therapeutic agents) are administered to a subject with a hyperproliferative cell disorder, especially cancer. A first therapy (e.g., prophylactic and/or therapeutic agent) can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., prophylactic and/or therapeutic agent) to a subject which had, has, or is susceptible to a hyperproliferative cell disorder, especially cancer. The therapies (e.g., prophylactic and/or therapeutic agents) are administered to a subject in a sequence and within a time interval such that the agent of the invention can act together with the other agent to provide an increased benefit than if they were administered otherwise. Any additional therapy (e.g., prophylactic and/or therapeutic agent) can be administered in any order with the other additional therapy (e.g., prophylactic and/or therapeutic agent).

As used herein, the phrase “low tolerance” refers to a state in which the patient suffers from side effects from treatment so that the patient does not benefit from and/or will not continue therapy because of the adverse effects and/or the harm from the side effects outweighs the benefit of the treatment.

As used herein, the terms “manage,” “managing” and “management” refer to the beneficial effects that a subject derives from administration of a therapy (e.g., prophylactic and/or therapeutic agent), which does not result in a cure of the disease. In certain embodiments, a subject is administered one or more therapies (e.g., prophylactic and/or therapeutic agents) to “manage” a disease so as to prevent the progression or worsening of the disease.

As used herein, the term “neoplastic” refers to a disease involving cells that have the potential to metastasize to distal sites and exhibit phenotypic traits that differ from those of non-neoplastic cells, for example, formation of colonies in a three-dimensional substrate such as soft agar or the formation of tubular networks or weblike matrices in a three-dimensional basement membrane or extracellular matrix preparation, such as MATRIGEL™. Non-neoplastic cells do not form colonies in soft agar and form distinct sphere-like structures in three-dimensional basement membrane or extracellular matrix preparations. Neoplastic cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless replicative potential, and sustained angiogenesis. Thus, “non-neoplastic” means that the condition, disease, or disorder does not involve cancer cells.

As used herein, the phrase “non-responsive/refractory” is used to describe patients treated with one or more currently available therapies (e.g., cancer therapies) such as chemotherapy, radiation therapy, surgery, hormonal therapy and/or biological therapy/immunotherapy, particularly a standard therapeutic regimen for the particular cancer, wherein the therapy is not clinically adequate to treat the patients such that these patients need additional effective therapy, e.g., remain unsusceptible to therapy. The phrase can also describe patients who respond to therapy yet suffer from side effects, relapse, develop resistance, etc. In various embodiments, “non-responsive/refractory” means that at least some significant portion of the cancer cells are not killed or their cell division arrested. The determination of whether the cancer cells are “non-responsive/refractory” can be made either in vivo or in vitro by any method known in the art for assaying the effectiveness of treatment on cancer cells, using the art-accepted meanings of “refractory” in such a context. In various embodiments, a cancer is “non-responsive/refractory” where the number of cancer cells has not been significantly reduced, or has increased during the treatment.

As used herein, the term “overexpress” in the context of EphA2 overexpression means that the gene encoding EphA2 is expressed at a level above that which is expressed by a normal human cell as assessed by an assay described herein or known to one of skill in the art (e.g., an immunoassay such as an ELISA or Western blot, a Northern blot, or RT-PCR).

As used herein, the term “potentiate” refers to an improvement in the efficacy of a therapy at its common or approved dose.

As used herein, the terms “prevent,” “preventing” and “prevention” refer to the prevention of the onset, recurrence, or spread of a disease in a subject resulting from the administration of a therapy (e.g., prophylactic or therapeutic agent), or a combination of therapies.

As used herein, the term “prophylactic agent” refers to any agent that can be used in the prevention of the onset, recurrence or spread of a disorder associated with EphA2 overexpression, a disorder associated with aberrant angiogenesis and/or a hyperproliferative cell disease, particularly cancer. In certain embodiments, the term “prophylactic agent” refers to a Listeria-based EphA2 vaccine of the invention. In certain other embodiments, the term “prophylactic agent” refers to a therapy other than a Listeria-based EphA2 vaccine, e.g., a cancer chemotherapeutic, radiation therapy, hormonal therapy, biological therapy (e.g., immunotherapy). In other embodiments, more than one prophylactic agent may be administered in combination.

As used herein, a “prophylactically effective amount” refers to that amount of a therapy (e.g., a prophylactic agent) sufficient to result in the prevention of the onset, recurrence or spread of a disorder (e.g., a disorder associated with aberrant angiogenesis and a hyperproliferative cell disease, preferably, cancer). A prophylactically effective amount may refer to the amount of therapy (e.g., a prophylactic agent) sufficient to prevent the onset, recurrence or spread of a disorder (e.g., a disorder associated with aberrant angiogenesis and a hyperproliferative cell disease, particularly cancer) in a subject including, but not limited to, subjects predisposed to a hyperproliferative cell disease, for example, those genetically predisposed to cancer or previously exposed to carcinogens. A prophylactically effective amount may also refer to the amount of a therapy (e.g., prophylactic agent) that provides a prophylactic benefit in the prevention of a disorder (e.g., a disorder associated with aberrant angiogenesis and a hyperproliferative cell disease). Further, a prophylactically effective amount with respect to a therapy (e.g., prophylactic agent) means that amount of a therapy (e.g., prophylactic agent) alone, or in combination with other therapies (e.g., agents), that provides a prophylactic benefit in the prevention of a disorder (e.g., a disorder associated with aberrant angiogenesis and a hyperproliferative cell disease). Used in connection with an amount of a Listeria-based EphA2 vaccine of the invention, the term can encompass an amount that improves overall prophylaxis or enhances the prophylactic efficacy of or synergies with another therapy (e.g., a prophylactic agent).

A used herein, a “protocol” includes dosing schedules and dosing regimens.

As used herein, the phrase “side effects” encompasses unwanted and adverse effects of a prophylactic or therapeutic agent. Adverse effects are always unwanted, but unwanted effects are not necessarily adverse. An adverse effect from a therapy (e.g., a prophylactic or therapeutic agent) might be harmful or uncomfortable or risky. Side effects from chemotherapy include, but are not limited to, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (56^{th} ed., 2002, 57^{th} ed., 2003 and 58^{th} ed., 2004).

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human. In a specific embodiment, the subject is a non-human animal. In another embodiment, the subject is a farm animal (e.g., a horse, a pig, a lamb or a cow) or a pet (e.g., a dog, a cat, a rabbit or a bird). In another embodiment, the subject is an animal other than a laboratory animal or animal model (e.g., a mouse, a rat, a guinea pig or a monkey). In a preferred embodiment, the subject is a human. In another preferred embodiment, the subject is a human that is not immunocompromised or immunosuppressed. In another preferred embodiment, the subject is a human with a mean absolute lymphocyte count of approximately 500 cells/mm³, approximately 600 cells/mm³, approximately 650 cells/mm³, approximately 700 cells/mm³, approximately 750 cells/mm³, approximately 800 cells/mm³, approximately 850 cells/mm³, approximately 900 cells/mm³, approximately 950 cells/mm³, approximately 1000 cells/mm³, approximately 1050 cells/mm³, approximately 1100 cells/mm³, or approximately 1150 cells/mm³ or approximately 1200 cells/mm³.

As used herein, the terms “treat,” “treating” and “treatment” refer to the eradication, reduction or amelioration of a disorder or a symptom thereof, particularly, the eradication, removal, modification, or control of primary, regional, or metastatic cancer tissue that results from the administration of one or more therapies (e.g., therapeutic agents). In certain embodiments, such terms refer to the minimizing or delaying the spread of cancer resulting from the administration of one or more therapies (e.g., therapeutic agents) to a subject with such a disease.

As used herein, the term “therapeutic agent” refers to any agent that can be used in the prevention, treatment, or management of a disease (e.g., a disorder associated with overexpression of EphA2 and/or hyperproliferative cell disorder, particularly, cancer). In certain embodiments, the term “therapeutic agent” refers to a Listeria-based EphA2 vaccine of the invention. In certain other embodiments, the term “therapeutic agent” refers to a therapy other than a Listeria-based EphA2 vaccine such as, e.g., a cancer chemotherapeutic, radiation therapy, hormonal therapy, and/or biological therapy/immunotherapy. In other embodiments, more than one therapy (e.g., a therapeutic agent) may be administered in combination.

As used herein, a “therapeutically effective amount” refers to that amount of a therapy (e.g., a therapeutic agent) sufficient to treat or manage a disorder (e.g., a disorder associated with EphA2 overexpression, a disorder associated with aberrant angiogenesis and/or hyperproliferative cell disease) and, preferably, the amount sufficient to destroy, modify, control or remove primary, regional or metastatic cancer tissue. A therapeutically effective amount may refer to the amount of a therapy (e.g., a therapeutic agent) sufficient to delay or minimize the onset of a disorder (e.g., hyperproliferative cell disease), e.g., delay or minimize the spread of cancer. A therapeutically effective amount may also refer to the amount of a therapy (e.g., a therapeutic agent) that provides a therapeutic benefit in the treatment or management of a disorder (e.g., cancer). Further, a therapeutically effective amount with respect to a therapy (e.g., a therapeutic agent) means that amount of a therapy (e.g., therapeutic agent) alone, or in combination with other therapies, that provides a therapeutic benefit in the treatment or management of a disorder (e.g., a hyperproliferative cell disease such as cancer). Used in connection with an amount of a Listeria-based EphA2 vaccine, the term can encompass an amount that improves overall therapy, reduces or avoids unwanted effects, or enhances the therapeutic efficacy of or synergies with another therapy (e.g., a therapeutic agent).

As used herein, the term “therapy” refers to any protocol, method and/or agent that can be used in the prevention, treatment or management of a disorder (e.g., a hyperproliferative cell disorder, a disorder associated with aberrant angiogenesis and/or a non-neoplastic hyperproliferative cell disorder) or a symptom thereof. In certain embodiments, the terms “therapies” and “therapy” refer to a biological therapy, supportive therapy, and/or other therapies useful in treatment, management, prevention, or amelioration of a disorder (e.g., a hyperproliferative cell disorder and/or a non-neoplastic hyperproliferative cell disorder) or one or more symptoms thereof known to one of skill in the art such as medical personnel.

As used herein, the term “synergistic” refers to a combination of therapies (e.g., prophylactic or therapeutic agents) which is more effective than the additive effects of any two or more single therapies (e.g., one or more prophylactic or therapeutic agents). A synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with a non-neoplastic hyperproliferative epithelial and/or endothelial cell disorder. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of a disorder (e.g., a hyperproliferative cell disorder). In addition, a synergistic effect can result in improved efficacy of therapies (e.g., prophylactic or therapeutic agents) in the prevention or treatment of a disorder (e.g., a disorder associated with aberrant angiogenesis and a hyperproliferative cell disorder). Finally, synergistic effect of a combination of therapies (e.g., prophylactic or therapeutic agents) may avoid or reduce adverse or unwanted side effects associated with the use of any single therapy.

As used herein, the terms “T cell malignancies” and “T cell malignancy” refer to any T cell lymphoproliferative disorder, including thymic and post-thymic malignancies. T cell malignancies include tumors of T cell origin. T cell malignancies refer to tumors of lymphoid progenitor cell, thymocyte, T cell, NK-cell, or antigen presenting cell origin. T cell malignancies include, but are not limited to, leukemias, including acute lymphoblastic leukemias, thymomas, acute lymphoblastic leukemias, and lymphomas, including Hodgkin's and non-Hodgkin's disease, with the proviso that T cell malignancies are not cutaneous T cell malignancies, in particular cutaneous-cell lymphomas. In a preferred embodiment, T cell malignancies are systemic, non-cutaneous T cell malignancies.

### 3.2. Sequences

Below is a brief summary of the sequences presented in the accompanying sequence listing, which is incorporated by reference herein in its entirety:

SEQ ID NO:1
Human EphA2 cDNA (full length)

SEQ ID NO:2
Human EphA2 polypeptide (full length)

SEQ ID NOs:3-18

Human EphA2 peptides

SEQ ID NO:19
Construct: LLOss-PEST-hEphA2
Native LLO signal peptide+PEST fused to full-length human EphA2
Not Codon optimized
No epitope tags (e.g., myc or FLAG used in this construct)
Fusion protein coding sequence shown

SEQ ID NO:20
Construct: LLOss-PEST-hEphA2
Native LLO signal peptide+PEST fused to full-length human EphA2
Not Codon optimized
No epitope tags (e.g., myc or FLAG used in this construct)
Predicted fusion protein shown

SEQ ID NO:21
EphA2 EX2 domain
Native nucleotide sequence

SEQ ID NO:22
EphA2 EX2 domain
Nucleotide sequence for optimal codon usage in Listeria

SEQ ID NO:23
EphA2 EX2 domain
Primary Amino Acid Sequence

SEQ ID NO:24
Construct: LLOss-PEST-EX2_hEphA2
Native LLO signal peptide+PEST fused to external domain of human EphA2
Not Codon optimized
No epitope tags (e.g., myc or FLAG used in this construct)

SEQ ID NO:25
Construct: LLOss-PEST-EX2_hEphA2
Native LLO signal peptide+PEST fused to external domain of human EphA2
Not Codon optimized
No epitope tags (e.g., myc or FLAG used in this construct)
Predicted fusion protein shown

SEQ ID NO:26
NativeLLOss-PEST-FLAG-EX2_EphA2-myc-CodonOp
(Native L. monocytogenes LLO signal peptide+PEST-Codon optimized-FLAG-EX-2 EphA2-Myc)
Nucleotide Sequence (including hly promoter)

SEQ ID NO:27
NativeLLOss-PEST-FLAG-EX2_EphA2-myc-CodonOp
(Native L. monocytogenes LLO signal peptide+PEST-Codon optimized-FLAG-EX-2 EphA2-Myc)
Primary Amino Acid Sequence

SEQ ID NO:28
Codon Optimized LLOss-PEST-FLAG-EX2_EphA2-myc-CodonOp
(Codon Optimized L. monocytogenes LLO signal peptide+PEST-Codon optimized-FLAG-EX-2 EphA2-Myc)
Nucleotide Sequence (including hly promoter)

SEQ ID NO:29
Codon Optimized LLOss-PEST-FLAG-EX2_EphA2-myc-CodonOp
(Codon Optimized L. monocytogenes LLO signal peptide+PEST-Codon optimized-FLAG-EX-2 EphA2-Myc)
Primary Amino Acid Sequence

SEQ ID NO:30
PhoD-FLAG-EX2_EphA2-myc-CodonOp
(Codon optimized B. subtilis phoD Tat signal peptide-FLAG-EX-2 EphA2-Myc)
Nucleotide Sequence (including hly promoter)

SEQ ID NO:31
PhoD-FLAG-EX2_EphA2-myc-CodonOp
(Codon optimized B. subtilis phoD Tat signal peptide-FLAG-EX-2 EphA2-Myc)
Amino acid sequence

SEQ ID NO:32
EphA2 CO domain
Native nucleotide sequence

SEQ ID NO:33
EphA2 CO domain
Nucleotide sequence for optimal codon usage in Listeria

SEQ ID NO:34
EphA2 CO domain
Primary Amino Acid Sequence

SEQ ID NO:35
Construct: LLOss-PEST-CO-huEphA2
Native LLO signal peptide+PEST fused to cytoplasmic domain of human EphA2
Not Codon optimized
No epitope tags (e.g., myc or FLAG used in this construct)
Fusion protein coding sequence shown

SEQ ID NO:36
Construct: LLOss-PEST-CO-huEphA2
Native LLO signal peptide+PEST fused to cytoplasmic domain of human EphA2
Not Codon optimized
No epitope tags (e.g., myc or FLAG used in this construct)
Predicted fusion protein shown

SEQ ID NO:37
NativeLLOss-PEST-FLAG-CO_EphA2-myc-CodonOp
(Native L. monocytogenes LLO signal peptide+PEST-Codon optimized-FLAG-CO_EphA2-Myc)
Nucleotide Sequence (including hly promoter)

SEQ ID NO:38
NativeLLOss-PEST-FLAG-CO_EphA2-myc-CodonOp
(Native L. monocytogenes LLO signal peptide+PEST-Codon optimized-FLAG-CO_EphA2-Myc)
Primary Amino Acid Sequence

SEQ ID NO:39
Codon Optimized LLOss-PEST-FLAG-CO_EphA2-myc-CodonOp
(Codon Optimized L. monocytogenes LLO signal peptide+PEST-Codon optimized-FLAG-CO_EphA2-Myc)
Nucleotide Sequence (including hly promoter)

SEQ ID NO:40
Codon Optimized LLOss-PEST-FLAG-CO_EphA2-myc-CodonOp
(Codon Optimized L. monocytogenes LLO signal peptide+PEST-Codon optimized-FLAG-CO_EphA2-Myc)
Primary Amino Acid Sequence

SEQ ID NO:41
PhoD-FLAG-CO_EphA2-myc-CodonOp
(Codon optimized B. subtilis phoD Tat signal peptide-FLAG-CO_EphA2-Myc)
Nucleotide Sequence (including hly promoter)

SEQ ID NO:42
PhoD-FLAG-CO_EphA2-myc-CodonOp
(Codon optimized B. subtilis phoD Tat signal peptide-FLAG-CO_EphA2-Myc)
Amino acid sequence

SEQ ID NO:43
Construct: pAM401-MCS
Plasmid pAM401 containing multiple cloning site (MCS) from pPL2 vector
Insertion of small Aat II MCS fragment from pPL2 inserted into pAM401 plasmid between blunted Xba I and Nru I sites.
Complete pAM401-MCS plasmid sequence shown

### 4. BRIEF DESCRIPTION OF THE FIGURES

FIG. 1. Listeria intracellular life cycle, antigen presenting cell activation, and antigen presentation.

FIG. 2. Western blot analysis of secreted protein from recombinant Listeria encoding native EphA2 CO domain sequence.

FIG. 3. Western blot analysis of secreted protein from recombinant Listeria encoding native or codon-optimized LLO secA1 signal peptide fused with codon-optimized EphA2 EX2 domain sequence signal peptide.

FIG. 4. Western blot analysis of secreted protein from recombinant Listeria encoding native or codon-optimized LLO secA2 signal peptide or codon-optimized Tat signal peptide fused with codon-optimized EphA2 CO domain sequence.

FIG. 5. Flow cytometry analysis of human EphA2 expression in CT2 murine carcinoma cells. Single cell FACS sorting assays were performed by standard techniques to identify CT26 cell clones expressing high levels of human EphA2.

FIG. 6. Western blot analysis of pooled populations CT26 murine colon carcinoma cells expressing high levels of human EphA2 protein.

FIG. 7. Flow Cytometry of B16F10 cells expressing huEphA2.

FIG. 8. Western blot analysis of lysate from 293 cells 48 hr. following transfection with pCDNA4 plasmid DNA encoding full-length native EphA2 sequence.

FIGS. 9A-9B. In the CT26 tumor model, therapeutic immunization with positive control Listeria expressing AH1-A5.

FIGS. 10A-10B. Preventative immunization with Listeria expressing ECD of hEphA2 suppresses CT26-hEphA2 tumor growth (FIG. 10A) and increases survival (FIG. 10B).

FIGS. 11A-11D. Preventive studies following i.v. administration of L4029EphA2-exFlag, Listeria control (L4029), or Listeria positive control containing the AH1 protein (L4029-AH1) (5×10⁵ cells in 100 μl volume) either subcutaneously or intravenously. FIG. 11A demonstrates tumor volume of mice inoculated with CT26 cells expressing the ECD of huEphA2, vehicle (HBSS), Listeria (L4029) or Listeria positive (L4029-AH1) controls. FIG. 11B demonstrates mean tumor volume of mice inoculated with CT26 cells expressing the ECD of huEphA2 (L4029-EphA2 exFlag) compared to the Listeria (L4029) control. FIG. 11C illustrates results of the prevention study in the s.c. model, measuring percent survival of the mice post CT26 tumor cell inoculation. FIG. 11D illustrates the results of the prevention study in the lung metastases model, measuring percent survival of the mice post tumor cell inoculation.

FIG. 12. Preventative immunization with Listeria expressing ECD of hEphA2 increases survival following RenCa-hEphA2 tumor challenge.

FIGS. 13A-13C. FIGS. 13A-13C illustrate results of a typical therapeutic study of animals inoculated with CT26 murine colon carcinoma cells transfected with human EphA2 (L4029-EphA2 exFlag), Listeria control (L4029-control) or vehicle (HBSS). In FIG. 13A, tumor volume was measured at several intervals post inoculation. FIG. 13B illustrates the mean tumor volume of mice inoculated with CT26 cells containing either Listeria control or the ECD of huEphA2. FIG. 13C represents the results of a therapeutic study using the lung metastases model, measuring percent survival of mice post inoculation with CT26 cells with either HBSS or Listeria control, or Listeria expressing the ECD of huEphA2.

FIGS. 14A-F. FIG. 14A. Therapeutic immunization in Balb/C mice with Listeria expressing ICD of hEphA2 suppresses established CT26-hEphA2 tumor growth. FIG. 14B. Immunization of Balb/C mice bearing CT26.24 (huEphA2+) lung tumors with recombinant Listeria encoding EphA2 CO domain confers long-term survival. FIG. 14C. Long-term survival of Balb/C mice bearing CT26.24 (huEphA2+) lung tumors immunized with recombinant Listeria encoding OVA.AH1 or OVA.AH1-A5. FIG. 14D. Increased survival of Balb/C mice bearing CT26.24 (huEphA2+) lung tumors when immunized with recombinant Listeria encoding codon-optimized or native EphA2 CO domain sequence. FIG. 14E. Increased survival of Balb/C mice bearing CT26.24 (huEphA2+) lung tumors when immunized with recombinant Listeria encoding codon-optimized secA1 signal peptide fused with codon-optimized EphA2 EX2 domain sequence. FIG. 14F. Immunization of Balb/C mice bearing CT26.24 (huEphA2+) lung tumors with recombinant Listeria encoding EphA2 CO domain but not with plasmid DNA encoding full-length EphA2 confers long-term survival.

FIG. 15. Long-term suppression of CT26-hEphA2 tumor growth upon rechallenge.

FIG. 16. Immunization with Listeria expressing hEphA2 elicits a specific CD8+ T cell response.

FIG. 17. Both CD4+ and CD8+ T cell responses are required for optimal hEphA2-directed anti-tumor efficacy.

FIGS. 18A-B. Therapeutic vaccination with Listeria expressing human EphA2 ICD enhances CD45+ tumor infiltrate. FIG. 18A depicts images of tumor sections stained with biotinylated rat anti-mouse CD45/B200. FIG. 18B is a bar graph normalizing the image data to tumor volume.

### 5. DETAILED DESCRIPTION OF THE INVENTION

The present invention is based, in part, on the inventors' discovery that a Listeria-based vaccine comprising Listeria engineered to express EphA2 antigenic peptides can confer beneficial therapeutic and prophylactic benefits against hyperproliferative diseases involving EphA2-expressing cells.

The present invention provides methods and compositions that provide for the prevention, treatment, inhibition, and management of disorders associated with overexpression of EphA2, disorders associated with aberrant angiogenesis and/or hyperproliferative cell disorders. A particular aspect of the invention relates to methods and compositions containing compounds that, when administered to a subject with a hyperproliferative cell disorder involving EphA2-expressing cells, either elicit or mediate an immune response against EphA2, resulting in a growth inhibition of the EphA2-expressing cells involved in the hyperproliferative cell disorder. The present invention further relates to methods and compositions for the treatment, inhibition, or management of metastases of cancers of epithelial cell origin, especially human cancers of the breast, ovarian, esophageal, lung, skin, prostate, bladder, and pancreas, and renal cell carcinomas and melanomas. The invention further relates to methods and compositions for the prevention, treatment, inhibition, or management of cancers of T cell origin, especially leukemias and lymphomas. Further, the compositions and methods of the invention include other types of active ingredients in combination with the Listeria-based EphA2 vaccines of the invention. In certain embodiments, the compositions of the invention are used to treat, prevent or manage other non-neoplastic hyperproliferative cell disorders, for example, but not limited to asthma, psoriasis, restenosis, COPD, etc.

The present invention also relates to methods for the treatment, inhibition, and management of cancer and other hyperproliferative cell disorders that have become partially or completely refractory to current or standard therapy (e.g., a cancer therapy, such as chemotherapy, radiation therapy, hormonal therapy, and biological/immunotherapy).

### 5.1. Listeria -Based Vaccines

The present invention provides Listeria bacteria engineered to express an EphA2 antigenic peptide and the use of such Listeria to manage, treat or prevent diseases associated with overexpression of EphA2 and/or hyperproliferative cell disorders.

A Listeria-based EphA2 vaccine may comprise one or more strains of Listeria that express an EphA2 antigenic peptide. In other embodiments, a Listeria-based EphA2 vaccine may comprise a Listeria strain that has been engineered to express one or more EphA2 antigenic peptides.

In a preferred embodiment, the Listeria-based EphA2 vaccine of the invention comprises the species Listeria monocytogenes.

5.1.1. Attenuation

To allow the safe use of Listeria in treatment of humans and animals, the bacteria are preferably attenuated in their virulence for causing disease. The end result is to reduce the risk of toxic shock or other side effects due to administration of the Listeria to the patient. Such attenuated Listeria can be isolated by a number of techniques. Such methods include use of antibiotic-sensitive strains of microorganisms, mutagenesis of the microorganisms, selection for microorganism mutants that lack virulence factors, and construction of new strains of microorganisms with altered cell wall lipopolysaccharides.

In certain embodiments, the Listeria can be attenuated by the deletion or disruption of DNA sequences which encode for virulence factors which insure survival of the Listeria in the host cell, especially macrophages and neutrophils, by, for example, homologous recombination techniques and chemical or transposon mutagenesis. Many, but not all, of these studied virulence factors are associated with survival in macrophages such that these factors are specifically expressed within macrophages due to stress, for example, acidification, or are used to induced specific host cell responses, for example, macropinocytosis, Fields et al., 1986, Proc. Natl. Acad. Sci. USA 83:5189-5193. Examples of virulence genes include, but are not limited to, hly, plcA, plcB, mpl, actA, inlA, and inlB. See also Autret et al., 2001, Infection and Immunity 69:2054-2065.

In a specific embodiment, the Listeria are attenuated in their tissue tropism (e.g., inlB mutant) and/or in their ability to spread from cell to cell (e.g., actA mutant). In another embodiment, the Listeria comprise a mutation (e.g., a deletion, addition or substitution) in one or more internalins (e.g., inlA and/or inlB). In another embodiment, the Listeria comprise a mutation (e.g., a deletion, addition or substitution) in actA.

As a method of insuring the attenuated phenotype and to avoid reversion to the non-attenuated phenotype, the Listeria may be engineered such that it is attenuated in more than one manner, e.g., a mutation affecting tissue tropism (e.g., inlB mutant) and a mutation affecting the ability to spread from cell to cell (e.g., actA mutant). In a preferred embodiment, the Listeria comprise a mutation (e.g., a deletion, addition or substitution) in internalin B and a mutation in actA.

5.1.2. Expression Systems

The EphA2 antigenic peptides are preferably expressed in Listeria using a heterologous gene expression cassette. A heterologous gene expression cassette is typically comprised of the following ordered elements: (1) prokaryotic promoter; (2) Shine-Dalgarno sequence; (3) secretion signal (signal peptide); and, (4) heterologous gene. Optionally, the heterologous gene expression cassette may also contain a transcription termination sequence, in constructs for stable integration within the bacterial chromosome. While not required, inclusion of a transcription termination sequence as the final ordered element in a heterologous gene expression cassette may prevent polar effects on the regulation of expression of adjacent genes, due to read-through transcription.

The expression vectors introduced into the Listeria-based EphA2 vaccine are preferably designed such that the Listeria-produced EphA2 peptides and, optionally, a second tumor antigen, are secreted by the Listeria. A number of bacterial secretion signals are well known in the art and may be used in the compositions and methods of the present invention. An exemplary secretion signals that can be used with Listeria is SecA, as described in Section 5.2.1.4, infra.

The promoters driving the expression of the EphA2 antigenic peptides may be either constitutive, in which the peptides are continually expressed; inducible, in which the peptides are expressed only upon the presence of an inducer molecule(s); or cell-type specific, in which the peptides or enzymes are expressed only in certain cell types.

Preferred embodiments of components of the EphA2 antigenic peptide expression system, to be used in conjunction with nucleic acids encoding EphA2 antigenic peptides described in Section 5.2, are provided below.

5.1.2.1. Construct Backbone

One of ordinary skill in the art will recognize that a variety of plasmid construct backbones are available which are suitable for use in the assembly of a heterologous gene expression cassette. A particular plasmid construct backbone is selected based on whether expression of the heterologous gene expression cassette from the bacterial chromosome or from an extra-chromosomal episome is desired.

Given as non-limiting examples, incorporation of the heterologous gene expression cassette into the bacterial chromosome of Listeria monocytogenes (Listeria) is accomplished with an integration vector that contains an expression cassette for a listeriophage integrase that catalyzes sequence-specific integration of the vector into the Listeria chromosome. For example, the integration vectors known as pPL1 and pPL2 program stable single-copy integration of a heterologous protein (e.g., EphA2-antigenic peptide) expression cassette within an innocuous region of the bacterial genome, and have been described in the literature (Lauer et al., 2002, J. Bacteriol. 184:4177-4178). The integration vectors are stable as plasmids in E. coli and are introduced via conjugation into the desired Listeria background. Each vector lacks a Listeria-specific origin of replication and encodes a phage integrase, such that the vectors are stable only upon integration into a chromosomal phage attachment site. Starting with a desired plasmid construct, the process of generating a recombinant Listeria strain expressing a desired protein(s) takes approximately one week. The pPL1 and pPL2 integration vectors are based, respectively, on the U153 and PSA listeriophages. The pPL1 vector integrates within the open reading frame of the comK gene, while pPL2 integrates within the tRNAArg gene in such a manner that the native sequence of the gene is restored upon successful integration, thus keeping its native expressed function intact. The pPL1 and pPL2 integration vectors contain a multiple cloning site sequence in order to facilitate construction of plasmids containing the heterologous protein (e.g., EphA2-antigenic peptide) expression cassette.

Alternatively, incorporation of the EphA2-antigenic peptide expression cassette into the Listeria chromosome can be accomplished through alleleic exchange methods, known to those skilled in the art. In particular, compositions in which it is desired to not incorporate a gene encoding an antibiotic resistance protein as part of the construct containing the heterologous gene expression cassette, methods of allelic exchange are desirable. For example, the pKSV7 vector (Camilli et al., 1993, Mol. Microbiol. 8:143-157), contains a temperature-sensitive Listeria Gram-positive replication origin which is exploited to select for recombinant clones at the non-permissive temperature that represent the pKSV7 plasmid recombined into the Listeria chromosome. The pKSV7 allelic exchange plasmid vector contains a multiple cloning site sequence in order to facilitate construction of plasmids containing the heterologous protein (e.g., EphA2-antigenic peptide) expression cassette, and also a chloramphenicol resistance gene. For insertion into the Listeria chromosome, the heterologous EphA2-antigenic peptide expression cassette construct is optimally flanked by approximately 1 kb of chromosomal DNA sequence that corresponds to the precise location of desired integration. The pKSV7-heterologous protein (e.g., EphA2-antigenic peptide) expression cassette plasmid is introduced optimally into a desired bacterial strain by electroporation, according to standard methods for electroporation of Gram positive bacteria. Briefly, bacteria electroporated with the pKSV7-heterologous protein (e.g., EphA2-antigenic peptide) expression cassette plasmid are selected by plating on BHI agar media containing chloramphenicol (10 μg/ml), and incubated at the permissive temperature of 30° C. Single cross-over integration into the bacterial chromosome is selected by passaging several individual colonies for multiple generations at the non-permissive temperature of 41° C. in media containing chloramphenicol. Finally, plasmid excision and curing (double cross-over) is achieved by passaging several individual colonies for multiple generations at the permissive temperature of 30° C. in BHI media not containing chloramphenicol. Verification of integration of the heterologous protein (e.g., EphA2-antigenic peptide) expression cassette into the bacteria chromosome can be accomplished by PCR, utilizing a primer pair that amplifies a region defined from within the heterologous protein (e.g., EphA2-antigenic peptide) expression cassette to the bacterial chromosome targeting sequence not contained in the pKSV7 plasmid vector construct.

In other compositions, it may be desired to express the heterologous protein (e.g., EphA2-antigenic peptide) from a stable plasmid episome. Maintenance of the plasmid episome through passaging for multiple generations requires the co-expression of a protein that confers a selective advantage for the plasmid-containing bacterium. As non-limiting examples, the protein co-expressed from the plasmid in combination with the heterologous protein (e.g., EphA2-antigenic peptide) may be an antibiotic resistance protein, for example chloramphenicol, or may be a bacterial protein (that is expressed from the chromosome in wild-type bacteria), that can also confer a selective advantage. Non-limiting examples of bacterial proteins include enzyme required for purine or amino acid biosynthesis (selection under defined media lacking relevant amino acids or other necessary precursor macromolecules), or a transcription factor required for the expression of genes that confer a selective advantage in vitro or in vivo (Gunn et al., 2001, J. Immuol. 167:6471-6479). As a non-limiting example, pAM401 is a suitable plasmid for episomal expression of a selected heterologous protein (e.g., EphA2-antigenic peptide) in diverse Gram-positive bacterial genera (Wirth et al., 1986, J. Bacteriol 165:831-836).

5.1.2.2. Shine-Dalgarno Sequence

At the 3′ end of the promoter is contained a poly-purine Shine-Dalgarno sequence, the element required for engagement of the 30S ribosomal subunit (via 16S rRNA) to the heterologous gene RNA transcript and initiation of translation. The Shine-Dalgarno sequence has typically the following consensus sequence (SEQ ID NO:66): 5′-NAGGAGGU-N5-10AUG (start codon)-3′. There are variations of the poly-purine Shine-Dalgarno sequence Notably, the Listeria hly gene that encodes listerolysin O (LLO) has the following Shine-Dalgarno sequence (SEQ ID NO:67): AAGGAGAGTGAAACCCATG (Shine-Dalgarno sequence is underlined, and the translation start codon is bolded).

5.1.2.3. Codon Optimization

In some embodiments, for optimal translation efficiency of a selected heterologous protein, it is desirable to utilize codons favored by Listeria. The preferred codon usage for bacterial expression can be determined as described in Nakamura et al., 2000, Nucl. Acids Res. 28:292. In some embodiments, codon-optimized expression of EphA2 antigenic peptides, from Listeria monocytogenes is desired.

The optimal codons utilized by Listeria monocytogenes for each amino acid are shown in Table 3 below.

TABLE 3
Listeria Codon Bias: Codons to be used for optimizing expression

| Amino Acid | One Letter Code | Optimal Listeria Codon |
|---|---|---|
| Alanine | A | GCA |
| Arginine | R | CGU |
| Asparagine | N | AAU |
| Aspartate | D | GAU |
| Cysteine | C | UGU |
| Glutamine | Q | CAA |
| Glutamate | E | GAA |
| Glycine | G | GGU |
| Histidine | H | CAU |
| Isoleucine | I | AUU |
| Leucine | L | UUA |
| Lysine | K | AAA |
| Methionine | M | AUG |
| Phenylalanine | F | UUU |
| Proline | P | CCA |
| Serine | S | AGU |
| Threonine | T | ACA |
| Tryptophan | W | UGG |
| Tyrosine | Y | UAU |
| Valine | V | GUU |

5.1.2.4. Signal Peptides

Bacteria utilize diverse pathways for protein secretion, including secA1 and Twin-Arg Translocation (Tat), which are located at the N-terminal end of the pre-protein. The majority of secreted proteins utilize the Sec pathway, in which the protein translocates through the bacterial membrane-embedded proteinaceous Sec pore in an unfolded conformation. In contrast, the proteins utilizing the Tat pathway are secreted in a folded conformation.

Nucleotide sequence encoding signal peptides corresponding to either of these protein secretion pathways (including, but not limited to, the signal peptides described in Section 5.1.2.4 and the signal and leader peptides described in Section 5.2.1) can be fused genetically in-frame to a desired heterologous protein coding sequence. The signal peptides optimally contain a signal peptidase at their carboxyl terminus for release of the authentic desired protein into the extra-cellular environment (Sharkov and Cai, 2002, J. Biol. Chem. 277:5796-5803; Nielsen et al., 1997, Protein Engineering 10:1-6). Signal peptide cleavage sites can be predicted using programs such as SignalP 3.0 (Bendtsen et al., 2004, J. Mol. Biol. 340:783-795. The signal peptides can be derived not only from diverse secretion pathways, but also from diverse bacterial genera. Signal peptides have a common structural organization, having a charged N-terminus (N-domain), a hydrophobic core region (H-domain) and a more polar C-terminal region (C-domain), however, they do not show sequence conservation. The C-domain of the signal peptide carries a type I signal peptidase (SPase I) cleavage site, having the consensus sequence A-X-A, at positions −1 and −3 relative to the cleavage site. Proteins secreted via the sec pathway have signal peptides that average 28 residues. Signal peptides related to proteins secreted by the Tat pathway have a tripartite organization similar to Sec signal peptides, but are characterized by having an RR-motif (R—R—X-#-#, where # is a hydrophobic residue), located at the N-domain/H-domain boundary. Bacterial Tat signal peptides average 14 amino acids longer than sec signal peptides. The Bacillus subtilis secretome may contain as many as 69 putative proteins that utilize the Tat secretion pathway, 14 of which contain a SPase I cleavage site (Jongbloed et al., 2002, J. Biol. Chem. 277:44068-44078; Thalsma et al., 2000, Microbiol. Mol. Biol. Rev. 64:515-547). Shown in Table 4 below are non-limiting examples of signal peptides that can be used in fusion compositions with a selected heterologous gene, resulting in secretion from the bacterium of the encoded protein.

TABLE 4
signal sequences useful for bacterial expression and secretion of EphA2.

| | | Signal pepidase | | | |
|---|---|---|---|---|---|
| Secretion | Signal Peptide Amino Acid | Site (cleavage site | | | |
| Pathway | Sequence (NH₂—CO₂) | represented by ‘) | Gene | Genus/species | SEQ ID NO: |
| secA1 | MKKIMLVFITLILVSLPI | TEA’KD | hly (LLO) | Listeria | 44 |
| | AQQTEAKD | (SEQ ID NO:70) | | monocytogene | |
| Tat | MTDKKSENQTEKTETK | DKA’LT | lmo0367 | Listeria | 45 |
| | ENKGMTRREMLKLSAV | (SEQ ID NO:71) | | monocytogenes | |
| | VVDQVDKALT | | | | |
| | MAYDSRFDEWVQKLK | VGA’FG | PhoD | Bacillus | 46 |
| | EESFQNNTFDRRKFIQG | (SEQ ID NO:72) | (alkaline | subtillis | |
| | AGKIAGLSLGLTIAQSV | | phosphate) | | |
| | GAFG | | | | |

There are a variety of proteins among diverse bacterial genera that are secreted via the Tat pathway. In some embodiments, selected Tat signal peptides corresponding to these proteins are fused genetically in-frame to a desired sequence encoding an EphA2 antigenic peptide, to facilitate secretion of the functionally linked Tat signal peptide-EphA2 protein chimera via the Tat pathway. Provided below are non-limiting examples of proteins from Bacillus subtilis and Listeria (innocua and monocytogenes) that are predicted to utilize Tat pathway secretion.

Putative Bacillus subtilis Proteins Secreted by Tat

>gi|2635523|emb|CAB15017.1| similar to two (component sensor histidine kinase (YtsA) (Bacillus subtilis)

>gi|2632548|emb|CAB12056.1| phosphodiesterase/alkaline phosphatase D (Bacillus subtilis)

>gi|2632573|emb|CAB12081.1| similar to hypothetical proteins (Bacillus subtilis)

>gi|2633776|emb|CAB13278.1| similar to hypothetical proteins (Bacillus subtilis)

>gi|2634674|emb|CAB14172.1| menaquinol:cytochrome c oxidoreductase (iron (sulfur subunit) (Bacillus subtilis)

>gi|2635595|emb|CAB15089.1| yubF (Bacillus subtilis)

>gi|2636361|emb|CAB15852.1| alternate gene name: ipa (29d˜similar to hypothetical proteins (Bacillus subtilis)

Putative Listeria Proteins Secreted by Tat

>gi|16799463|ref|NP_{—}469731.1| conserved hypothetical protein similar to B. subtilis YwbN protein (Listeria innocua)

>gi|16801368|ref|NP_{—}471636.1| similar to 3 (oxoacyl(acyl(carrier protein synthase (Listeria innocua)

Listeria monocytogenes EGD (e)

>gi|16802412|ref|NP_{—}463897.1| conserved hypothetical protein similar to B. subtilis YwbN protein (Listeria monocytogenes EGD (e)

Organisms utilize codon bias to regulate expression of particular endogenous genes. Thus, signal peptides utilized for secretion of selected heterologous proteins may not contain codons that utilize preferred codons, resulting in non-optimal levels of protein synthesis. In some some embodiments, the signal peptide sequence fused in frame with a gene encoding a selected heterologous protein is codon-optimized for codon usage in a selected bacterium. In some embodiments for expression and secretion from recombinant Listeria monocytogenes, a nucleotide sequence of a selected signal peptide is codon optimized for expression in Listeria monocytogenes, according to Table 4, supra.

5.1.2.5. Transcription Termination Sequence

In some embodiments, a transcription termination sequence can be inserted into the heterologous protein expression cassette, downstream from the C-terminus of the translational stop codon related to the heterologous protein. Appropriate sequence elements known to those who are skilled in the art that promote either rho-dependent or rho-independent transcription termination can be placed in the heterologous protein expression cassette.

### 5.2. EphA2 Antigenic Peptides

As discussed above, the present invention relates to the use of Listeria that have been engineered to express an EphA2 antigenic peptide. Without being bound by any mechanism, such Listeria are capable of eliciting an immune response to EphA2 upon administration to a subject with a disease involving overexpression of EphA2, resulting in a cellular or humoral immune response against endogenous EphA2.

In principle, an EphA2 antigenic peptide (sometimes referred to as an “EphA2 antigenic polypeptide”) for use in the methods and compositions of the present invention can be any EphA2 antigenic peptide that is capable of eliciting an immune response against EphA2-expressing cells involved in a hyperproliferative disorder. Thus, an EphA2 antigenic peptide can be an EphA2 polypeptide, preferably an EphA2 polypeptide of SEQ ID NO:2, or a fragment or derivative of an EphA2 polypeptide that (1) displays antigenicity of EphA2 (ability to bind or compete with EphA2 for binding to an anti-EphA2 antibody, (2) displays immunogenicity of EphA2 (ability to generate antibody which binds to EphA2), and/or (3) contains one or more T cell epitopes of EphA2.

In certain embodiments, the EphA2 antigenic peptide is a sequence encoded by the nucleotide sequence provided below or a fragment or derivative thereof:

| | |
|---|---|
| Genbank Accession No. NM_004431 | Human |
| Genbank Accession No. NM_010139 | Mouse |
| Genbank Accession No. AB038986 | Chicken (partial) |

In certain embodiments, the EphA2 antigenic peptide is full length human EphA2 (SEQ ID NO:2).

In other embodiments, the EphA2 antigenic peptide comprises the intracellular domain of EphA2 (residue 22 to 554 of SEQ ID NO:2).

In yet other embodiments, the EphA2 antigenic peptide comprises the instracellular domain EphA2 (residue 558 to 976 of SEQ ID NO:2).

In yet other embodiments, the EphA2 antigenic peptide comprises more than one domain of the full length human EphA2. In a specific embodiment, the EphA2 antigenic peptides comprises the extracellular domain and the intracellular cytoplasmic domain, joined together. In accordance with this embodiment, the transmembrane domain of EphA2 is deleted.

In certain embodiments of the invention, the tyrosine kinase activity of EphA2 is ablated. Thus, EphA2 may contain deletions, additions or substitutions of amino acid residues that result in the elimination of tyrosine kinase activity. In a preferred embodiment, a lysine to methione substitution at position 646 is present.

In a preferred embodiment, the EphA2 antigenic peptide comprises the extracellular and cytoplasmic domains of EphA2 resulting from a deletion of the transmembrane domain of EphA2 and has a lysine to methionine substitution as position 646.

In certain embodiments, the peptide corresponds to or comprises an EphA2 epitope that is exposed in a cancer cell but occluded in a non-cancer cell. In a preferred embodiment, the EphA2 antigenic peptides preferentially include epitopes on EphA2 that are selectively exposed or increased on cancer cells but not non-cancer cells (“exposed EphA2 epitope peptides”).

The present invention further encompasses the use of a plurality of EphA2 antigenic peptides, e.g., 2, 3, 4, 5, 6, or more EphA2 antigenic peptides, in the compositions and methods of the present invention.

Fragments of EphA2 that are useful in the methods and compositions present invention may contain deletions, additions or substitutions of amino acid residues within the amino acid sequence encoded by an EphA2 gene. Preferably mutations result in a silent change, thus producing a functionally equivalent EphA2 gene product. By “functionally equivalent”, it is meant that the mutated EphA2 gene product has the same function as the wild-type EphA2 gene product, e.g., contains one or more epitopes of EphA2.

An EphA2 antigenic peptide sequence preferably comprises an amino acid sequence that exhibits at least about 65% sequence similarity to human EphA2, more preferably exhibits at least 70% sequence similarity to human EphA2, yet more preferably exhibits at least about 75% sequence similarity human EphA2. In other embodiments, the EphA2 polypeptide sequence preferably comprises an amino acid sequence that exhibits at least 85% sequence similarity to human EphA2, yet more preferably exhibits at least 90% sequence similarity to human EphA2, and most preferably exhibits at least about 95% sequence similarity to human EphA2.

Additional polypeptides suitable in the present methods are those encoded by the nucleic acids described in Section 5.2 below.

The determination of percent identity between two sequences can be accomplished using a mathematical algorithm. A preferred, non-limiting example of a mathematical algorithm utilized for the comparison of two sequences is the algorithm of Karlin and Altschul, 1990, Proc Natl Acad Sci. USA 87:2264-2268, modified as in Karlin and Altschul, 1993, Proc Natl Acad Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used.

Another preferred, non limiting example of a mathematical algorithm utilized for the comparison of sequences is the algorithm of Myers and Miller, 1988, Comput Appl Biosci 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman,1988, Proc Natl Acad Sci USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2.

The percent identity between two sequences can be determined using techniques similar to those described above, with or without allowing gaps. In calculating percent identity, only exact matches are counted. However, conservative substitutions should be considered in evaluating sequences that have a low percent identity with the EphA2 sequences disclosed herein.

In a specific embodiment, EphA2 antigenic peptides comprising at least 10, 20, 30, 40, 50, 75, 100, or 200 amino acids of an EphA2 polypeptide, preferably of SEQ ID NO:2 are used in the present invention. In a preferred embodiment, EphA2 antigenic peptides comprising at least 10, 20, 30, 40, 50, 75, 100, or 200 continguous amino acids of an EphA2 polypeptide, preferably of SEQ ID NO:2 are used in the present invention. In a preferred embodiment, such a polypeptide comprises all or a portion of the extracellular domain of an EphA2 polypeptide of SEQ ID NO:2.

5.2.1. Fusion Proteins

In certain embodiments of the present invention, a Listeria-based EphA2 vaccine expresses an EphA2 antigenic peptide that is a fusion protein. Thus, the present invention encompasses compositions and methods in which the EphA2 antigenic peptides are fusion proteins comprising all or a fragment or derivative of EphA2 operatively associated to a heterologous component, e.g., a heterologous peptide. Heterologous components can include, but are not limited to sequences which facilitate isolation and purification of the fusion protein. Heterologous components can also include sequences which confer stability to EphA2 antigenic peptides. Such fusion partners are well known to those of skill in the art.

The present invention encompasses the use of fusion proteins comprising an EphA2 polypeptide (e.g., a polypeptide of SEQ ID NO:2 or a fragment thereof) and a heterologous polypeptide (i.e., a polypeptide or fragment thereof, preferably a fragment of at least 10, at least 20, at least 30, at least 40, at least 50, at least 60, at least 70, at least 80, at least 90 or at least 100 contiguous amino acids of the polypeptide). The fusion can be direct, but may occur through linker sequences. The heterologous polypeptide may be fused to the N-terminus or C-terminus of the EphA2 antigenic peptide. Alternatively, the heterologous polypeptide may be flanked by EphA2 polypeptide sequences

A fusion protein can comprise an EphA2 antigenic peptide fused to a heterologous signal sequence at its N-terminus. Various signal sequences are commercially available. In addition to the signal sequences described in Section 5.1.2.4 supra, prokaryotic heterologous signal sequences useful in the methods of the invention include, but are not limited to, the phoA secretory signal (Sambrook et al., eds., 1989, Molecular Cloning: A Laboratory Manual, 2nd ed., Cold Spring Harbor Laboratory, Cold Spring Harbor Laboratory Press, Cold Spring Harbor, N.Y.) and the protein A secretory signal (Pharmacia Biotech, Piscataway, N.J.).

The EphA2 antigenic peptide can be fused to tag sequences, e.g., a hexa-histidine peptide, such as the tag provided in a pQE vector (QIAGEN, Inc., Chatsworth, Calif.), among others, many of which are commercially available for use in the methods of the invention. As described in Gentz et al., 1989, Proc. Natl. Acad. Sci. USA, 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson et al., 1984, Cell, 37:767) and the “flag” tag (Knappik et al., 1994, Biotechniques, 17(4):754-761). These tags are especially useful for purification of recombinantly produced EphA2 antigenic peptides.

Any fusion protein may be readily purified by utilizing an antibody specific or selective for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972). In this system, the gene of interest is subcloned into a vaccinia recombination plasmid such that the open reading frame of the gene is translationally fused to an amino-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

An affinity label can also be fused at its amino terminal to the carboxyl terminal of the EphA2 antigenic peptide for use in the methods of the invention. The precise site at which the fusion is made in the carboxyl terminal is not critical. The optimal site can be determined by routine experimentation. An affinity label can also be fused at its carboxyl terminal to the amino terminal of the EphA2 antigenic peptide for use in the methods and compositions of the invention.

A variety of affinity labels known in the art may be used, such as, but not limited to, the immunoglobulin constant regions (see also Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels are recognized by specific binding partners and thus facilitate isolation by affinity binding to the binding partner which can be immobilized onto a solid support. Some affinity labels may afford the EphA2 antigenic peptide novel structural properties, such as the ability to form multimers. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or fragments of the immunoglobulin molecule containing sites for interchain disulfide bonds, could lead to the formation of multimers.

As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

Various leader sequences known in the art can be used for the efficient secretion of the EphA2 antigenic peptide from bacterial cells such as Listeria (von Heijne, 1985, J. Mol. Biol. 184:99-105). In addition to the signal sequences described above and in Section 5.1.2.4, suitable leader sequences for targeting EphA2 antigenic peptide expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (MacIntyre et al., 1990, Mol. Gen. Genet. 221:466-74; Kaiser et al., 1987, Science, 235:312-317).

In certain embodiments, the fusion partner comprises a non-EphA2 polypeptide corresponding to an antigen associated with the cell type against which a therapeutic or prophylactic immune is desired. For example, the non-EphA2 polypeptide can comprise an epitope of a tumor-associated antigen, such as, but not limited to, MAGE-1, MAGE-2, MAGE-3, gp100, TRP-2, tyrosinase, MART-1, β-HCG, CEA, Ras, β-catenin, gp43, GAGE-1, GAGE -2, N-acetylglucosaminyltransferase-V, p15, β-catenin, BAGE-1, PSA, MUM-1, CDK4, HER-2/neu, Human papillomavirus-E6, Human papillomavirus-E7, and MUC-1, 2, 3.

Polynucleotides encoding fusion proteins can be produced by standard recombinant DNA techniques. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence coding for a fusion protein can be inserted into an appropriate expression vector, i.e., a vector which contains the necessary elements for the transcription and translation of the inserted protein-coding sequence. The expression of a fusion protein may be regulated by a constitutive, inducible or tissue-specific or -selective promoter. It will be understood by the skilled artisan that fusion proteins, which can facilitate solubility and/or expression, and can increase the in vivo half-life of the EphA2 antigenic peptide and thus are useful in the methods of the invention. The EphA2 antigenic peptides or peptide fragments thereof, or fusion proteins can be used in any assay that detects or measures EphA2 antigenic peptides or in the calibration and standardization of such assay.

The methods of invention encompass the use of EphA2 antigenic peptides or peptide fragments thereof, which may be produced by recombinant DNA technology using techniques well known in the art. Thus, methods for preparing the EphA2 antigenic peptides of the invention by expressing nucleic acid containing EphA2 antigenic gene sequences are described herein. Methods which are well known to those skilled in the art can be used to construct expression vectors containing, e.g., EphA2 antigenic peptide coding sequences (including but not limited to nucleic acids encoding all or an antigenic portion of a polypeptide of SEQ ID NO:2) and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding EphA2 antigenic peptide sequences may be chemically synthesized using, for example, synthesizers (see, e.g., the techniques described in Oligonucleotide Synthesis, 1984, Gait, M. J. ed., IRL Press, Oxford).

In certain embodiments, the EphA2 antigenic peptide is functionally coupled to an internalization signal peptide, also referred to as a “protein transduction domain,” that would allow its uptake into the cell nucleus. In certain specific embodiments, the internalization signal is that of Antennapedia (reviewed by Prochiantz, 1996, Curr. Opin. Neurobiol. 6:629-634, Hox A5 (Chatelin et al., 1996, Mech. Dev. 55:111-117), HIV TAT protein (Vives et al., 1997, J. Biol. Chem. 272:16010-16017) or VP22 (Phelan et al., 1998, Nat. Biotechnol. 16:440-443).

5.2.2. Polynucleotides Encoding EphA2 Antigenic Peptides

The present invention also encompasses the use of Listeria-based vaccines that comprise or contain polynucleotides that hybridize under high stringency, intermediate or lower stringency hybridization conditions, e.g., as defined infra, to polynucleotides that encode an EphA2 antigenic peptide of the invention.

By way of example and not limitation, procedures using such conditions of low stringency for regions of hybridization of over 90 nucleotides are as follows (see also Shilo and Weinberg, 1981, Proc. Natl. Acad. Sci. USA 78:6789-6792). Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art (e.g., as employed for cross-species hybridizations).

Also, by way of example and not limitation, procedures using such conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography.

Other conditions of high stringency which may be used depend on the nature of the nucleic acid (e.g., length, GC content, etc.) and the purpose of the hybridization (detection, amplification, etc.) and are well known in the art. For example, stringent hybridization of a nucleic acid of approximately 15-40 bases to a complementary sequence in the polymerase chain reaction (PCR) is done under the following conditions: a salt concentration of 50 mM KCl, a buffer concentration of 10 mM Tris-HCl, a Mg²⁺ concentration of 1.5 mM, a pH of 7-7.5 and an annealing temperature of 55-60° C.

Selection of appropriate conditions for moderate stringencies is also well known in the art (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, © 1987-1997, Current Protocols, © 1994-1997 John Wiley and Sons, Inc.).

The nucleic acids useful in the present methods may be made by any method known in the art. For example, if the nucleotide sequence of the EphA2 antigenic peptide is known, a nucleic acid encoding the peptide may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the peptide, annealing and ligating of those oligonucleotides, and then amplification of the ligated oligonucleotides by PCR.

Alternatively, a polynucleotide encoding an EphA2 antigenic peptide may be generated from nucleic acid from a suitable source. If a clone containing a nucleic acid encoding a particular peptide is not available, but the sequence of the EphA2 antigenic peptide is known, a nucleic acid encoding the peptide may be chemically synthesized or obtained from a suitable source (e.g., a cDNA library, or a cDNA library generated from, or nucleic acid, preferably poly A+ RNA, isolated from, any tissue or cells expressing EphA2) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the peptide. Amplified nucleic acids generated by PCR may then be cloned into replicable cloning vectors using any method well known in the art.

Further, a nucleic acid that is useful in the present methods may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate EphA2 antigenic peptides having a different amino acid sequence from the amino acid sequence depicted in SEQ ID NO:2, for example to create amino acid substitutions, deletions, and/or insertions.

### 5.3. Assays for EphA2 Antigenic Peptides

The present invention provide Listeria-based EphA2 vaccines comprising Listeria bacteria engineered to express an EphA2 antigenic peptide. Any assay known in the art for determining whether a peptide is a T cell epitope or a B cell epitope may be employed in testing EphA2 peptides for suitability in the present methods and compositions.

For example, for determining whether a peptide is a T cell epitope, ELISPOT assays and methods for intracellular cytokine staining can be used for enumeration and characterization of antigen-specific CD4⁺ and CD8⁺ T cells. Lalvani et al. (1997) J. Exp. Med. 186:859-865; Waldrop et al. (1997) J. Clin Invest. 99:1739-1750.

EphA2 antigenic peptides can be determined by screening synthetic peptides corresponding to portions of EphA2. Candidate antigenic peptides can be identified on the basis of their sequence or predicted structure. A number of algorithms are available for this purpose.

Exemplary protocols for such assays are presented below.

5.3.1. Peptides that Display Immunogenicity of EphA2

The ability of EphA2 peptides to elicit EphA2-specific antibody responses in mammals can be examined, for example, by immunizing animals (e.g., mice, guinea pigs or rabbits) with individual EphA2 peptides emulsified in Freund's adjuvant.

After three injections (5 to 100 μg peptide per injection), IgG antibody responses are tested by peptide-specific ELISAs and immunoblotting against EphA2.

EphA2 peptides which produce antisera that react specifically with the EphA2 peptides and also recognized full length EphA2 protein in immunoblots are said to display the antigenicity of EphA2.

5.3.2. CD4⁺ T-Cell Proliferation Assay

For example, such assays include in vitro cell culture assays in which peripheral blood mononuclear cells (“PBMCs”) are obtained from fresh blood of a patient with a disease involving overexpression of EphA2, and purified by centrifugation using FICOLL-PLAQUE PLUS (Pharmacia, Upsalla, Sweden) essentially as described by Kruse and Sebald, 1992, EMBO J. 11:3237-3244. The peripheral blood mononuclear cells are indubated for 7-10 days with candidate EphA2 antigenic peptides. Antigen presenting cells may optionally be added to the culture 24 to 48 hours prior to the assay, in order to process and present the antigen. The cells are then harvested by centrifugation, and washed in RPMI 1640 media (GibcoBRL, Gaithersburg, Md.). 5×10⁴ activated T cells/well are in RPMI 1640 media containing 10% fetal bovine serum, 10 mM HEPES, ph 7.5, 2 mM L-glutamine, 100 units/ml penicillin G, and 100 μg/ml streptomycin sulphate in 96 well plates for 72 hrs at 37° C., pulsed with 1 μCi ³H-thymidine (DuPont NEN, Boston, Mass.)/well for 6 hrs, harvested, and radioactivity measured in a TOPCOUNT scintillation counter (Packard Instrument Col., Meriden, Conn.).

5.3.3. Intracellular Cytokine Staining (ICS)

Measurement of antigen-specific, intracellular cytokine responses of T cells can be performed essentially as described by Waldrop et al., 1997, J. Clin. Invest. 99:1739-1750; Openshaw et al., 1995, J. Exp. Med. 182:1357-1367; or Estcourt et al., 1997, Clin. Immunol. Immunopathol. 83:60-67. Purified PBMCs from patients with a disease involving EphA2-overexpressing cells are placed in 12×75 millimeter polystyrene tissue culture tubes (Becton Dickinson, Lincoln Park, N.J.) at a concentration of 1×10⁶ cells per tube. A solution comprising 0.5 milliliters of HL-1 serum free medium, 100 units per milliliter of penicillin, 100 units per milliliter streptomycin, 2 millimolar L glutamine (Gibco BRL), varying amounts of individual EphA2 antigenic candidate peptides, and 1 unit of anti-CD28 mAb (Becton-Dickinson, Lincoln Park, N.J.) is added to each tube. Anti-CD3 mAb is added to a duplicate set of normal PBMC cultures as positive control. Culture tubes are incubated for 1 hour. Brefeldin A is added to individual tubes at a concentration of 1 microgram per milliliter, and the tubes are incubated for an additional 17 hours.

PBMCs stimulated as described above are harvested by washing the cells twice with a solution comprising Dulbecco's phosphate-buffered saline (dPBS) and 10 units of Brefeldin A. These washed cells are fixed by incubation for 10 minutes in a solution comprising 0.5 milliliters of 4% paraformaldehyde and dPBS. The cells are washed with a solution comprising dPBS and 2% fetal calf serum (FCS). The cells are then either used immediately for intracellular cytokine and surface marker staining or are frozen for no more than three days in freezing medium, as described (Waldrop et al., 1997, J. Clin. Invest. 99:1739-1750).

The cell preparations were rapidly thawed in a 37° C. water bath and washed once with dPBS. Cells, either fresh or frozen, are resuspended in 0.5 milliliters of permeabilizing solution (Becton Dickinson Immunocytometry systems, San Jose, Calif.) and incubated for 10 minutes at room temperature with protection from light. Permeabilized cells are washed twice with dPBS and incubated with directly conjugated mAbs for 20 minutes at room temperature with protection from light. Optimal concentrations of antibodies are predetermined according to standard methods. After staining, the cells were washed, refixed by incubation in a solution comprising dPBS 1% paraformaldehyde, and stored away from light at 4° C. for flow cytometry analysis.

5.3.4. ELISPOT Assays

The ELISPOT assay measures Th1-cytokine specific induction in murine splenocytes following Listeria vaccination. ELISPOT assays are performed to determine the frequency of T lymphocytes in response to endogenous antigenic peptide stimulation, and are as described in Geginat et al., 2001, J. Immunol. 166:1877-1884. Balb/c mice (3 per group) are vaccinated with L. monocytogenes expressing candidate EphA2 antigenic peptides or HBSS as control. Whole mouse spleens are harvested and pooled five days after vaccination. Single cell suspensions of murine splenocytes are plated in the presence of various antigens overnight in a 37° C. incubator.

Assays are performed in nitrocellulose-backed 96-well microtiter plates coated with rat anti-mouse IFN-γ mAb. For the testing of the candidate EphA2 antigenic peptide, a 1×10⁻⁵ M peptide solution is prepared. In round-bottom 96-well microtiter plates per well 6×10⁵ unseparated splenocytes in 135 μl culture medium (a modification of Eagle's medium (Life Technologies, Eggenstein, Germany) supplemented with 10% FCS, 100 U/ml penicillin, 100 μg/ml streptomycin, 1×10⁻⁵ M 2-ME, and 2 mM glutamine) are mixed with 15 μl of the 1×10⁻⁵ M peptide solution to yield a final peptide concentration of 1×10⁻⁶M. After 6 h of incubation at 37° C., cells are resuspended by vigorous pipetting, and 100 μl or 10 μl of cell suspension (4×10⁵/well or 4×10⁴/well, respectively) is transferred to Ab-coated ELISPOT plates and incubated overnight at 37° C. In the ELISPOT plates, the final volume was adjusted to 150 μl to ensure homogenous distribution of cells.

Purified CD4⁺ or CD8⁺ T cells are tested in a modified assay as follows: 15 μl prediluted peptide (1×10⁻⁵ M) is directly added to Ab-coated ELISPOT plates and mixed with 4×10⁵ splenocytes from nonimmune animals as APC to yield a final volume of 100 μl. After 4 h of preincubation of APC at 37° C., 1×10⁵ CD4⁺ or CD8⁺ cells purified from L. monocytogenes-immune mice are added per well in a volume of 50 μl and plates are incubated overnight at 37° C. The ELISPOT-based ex vivo MHC restriction analysis is performed after loading of cell lines expressing specific MHC class I molecules with 1×10⁻⁶ M peptide for 2 h at 37° C. Subsequently, unbound peptides are washed off (four times) to prevent binding of peptides to responder splenocytes. Per well of the ELISPOT plate, 1×10⁵ peptide-loaded APC are mixed with 4×10⁵ or 4×10⁴ responder splenocytes in a final volume of 150 μl. After overnight incubation at 37° C., ELISPOT plates are developed with biotin-labeled rat anti-mouse IFN-γ mAb, HRP streptavidin conjugate, and aminoethylcarbazole dye of spots per splenocytes seeded. The specificity and sensitivity of the ELISPOT assay is controlled with IFN-γ secreting CD8 T cell lines specific for a control antigen.

### 5.4. Prophylactic/Therapeutic Methods

The present invention provides methods for treating, preventing, or managing a disorder associated with overexpression of EphA2 and/or hyperproliferative cell disorders, preferably cancer, comprising administering to a subject in need thereof one or more Listeria-based EphA2 vaccines of the invention.

The present invention encompasses methods for eliciting an immune response against an EphA2-expressing cell associated with a hyperproliferative cell disorder, comprising administering to a subject one or more Listeria-based EphA2 vaccines of the invention in an amount effective for eliciting an immune response against the EphA2-expressing cell.

In another specific embodiment, the disorder to be treated, prevented, or managed is a pre-cancerous condition associated with cells that overexpress EphA2. In more specific embodiments, the pre-cancerous condition is high-grade prostatic intraepithelial neoplasia (PIN), fibroadenoma of the breast, fibrocystic disease, or compound nevi.

The present invention provides methods for treating, preventing, or managing a disorder associated with overexpression of EphA2 and/or hyperproliferative cell disorders, preferably cancer, comprising administering to a subject in need thereof one or more Listeria-based EphA2 vaccines of the invention and one or more other therapies. Examples of other therapies include, but are not limited to, those listed below in Section 5.4.3, infra. In one embodiment, the Listeria-based EphA2 vaccine of the invention can be administered in combination with one or more other therapies (e.g., prophylactic or therapeutic agents) useful in the treatment, prevention or management of disorders associated with EphA2 overexpression and/or hyperproliferative cell disorders, such as cancer. In certain embodiments, one or more Listeria-based EphA2 vaccines are administered to a subject, preferably a human, concurrently with one or more other therapies (e.g., therapeutic agents) useful for the treatment or management of cancer. The term “concurrently” is not limited to the administration of therapies (e.g., prophylactic or therapeutic agents) at exactly the same time, but rather it is meant that a Listeria-based EphA2 vaccine of the invention and another therapy are administered to a subject in a sequence and within a time interval such that the Listeria-based EphA2 vaccine can act together with the other therapy to provide an increased benefit than if they were administered otherwise. For example, each therapy (e.g., prophylactic or therapeutic agent) may be administered at the same time or sequentially in any order at different points in time; however, if not administered at the same time, they should be administered sufficiently close in time so as to provide the desired therapeutic or prophylactic effect. Each therapy (e.g., prophylactic or therapeutic agent) can be administered separately, in any appropriate form and by any suitable route. In certain embodiments, the Listeria-based EphA2 vaccines of the invention are administered before, concurrently or after surgery. Preferably, the surgery completely removes localized tumors or reduces the size of large tumors. Surgery can also be done as a preventive measure or to relieve pain.

In various embodiments, the therapies (e.g., prophylactic or therapeutic agents) are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more therapies are administered within the same patient visit.

The dosage amounts and frequencies of administration provided herein are encompassed by the terms therapeutically effective and prophylactically effective. The dosage and frequency further will typically vary according to factors specific for each patient depending on the specific therapeutic or prophylactic agents administered, the severity and type of cancer, the route of administration, as well as age, body weight, response, and the past medical history of the patient. Suitable regimens can be selected by one skilled in the art by considering such factors and by following, for example, dosages reported in the literature and recommended in the Physician's Desk Reference (56^{th} ed., 2002, 57^{th} ed., 2003 and 58^{th} ed., 2004).

5.4.1.1. Patient Population

The invention provides methods for treating, preventing, and/or managing a disorder associated with EphA2 overexpression and/or hyperproliferative cell disease, particularly cancer, comprising administrating to a subject in need thereof one or more Listeria-based EphA2 vaccines of the invention in a therapeutically or prophylactically effective amount or an amount effective to elicit an immune response against EphA2-expressing cells associated with the hyperproliferative disorder. In another embodiment, an effective amount of a Listeria-based EphA2 vaccine of the invention is administered in combination with an effective amount of one or more other therapies (e.g., therapeutic or prophylactic agents) to treat, prevent, and/or manage a disorder associated with EphA2 overexpression and/or hyperproliferative cell disease, particularly cancer. The subject is preferably a mammal such as non-primate (e.g., cows, pigs, horses, cats, dogs, rats, etc.) and a primate (e.g., monkey, such as a cynomolgous monkey and a human). In a preferred embodiment, the subject is a human.

Specific examples of cancers that can be treated by the methods encompassed by the invention include, but are not limited to, cancers that overexpress EphA2. In one embodiment, the cancer is of an epithelial origin. Examples of such cancers are cancer of the lung, colon, prostate, breast, and skin. Other cancers include cancer of the bladder and pancreas and renal cell carcinoma and melanoma. In another embodiment, the cancer is a solid tumor. In another embodiment, the cancer is of a T cell origin. Examples of such cancers are leukemias and lymphomas. Additional cancers are listed by example and not by limitation in the following Section 5.4.1.1. In particular embodiments, methods of the invention can be used to treat and/or prevent metastasis from primary tumors.

The methods and compositions of the invention comprise the administration of one or more Listeria-based EphA2 vaccines of the invention to subjects/patients suffering from or expected to suffer from cancer, e.g., have a genetic predisposition for a particular type of cancer, have been exposed to a carcinogen, or are in remission from a particular cancer. As used herein, “cancer” refers to primary or metastatic cancers. Such patients may or may not have been previously treated for cancer. The methods and compositions of the invention may be used as any line of cancer therapy, e.g., a first line, second line or third line of cancer therapy. Included in the invention is also the treatment of patients undergoing other cancer therapies and the methods and compositions of the invention can be used before any adverse effects or intolerance of these other cancer therapies occurs. The invention also encompasses methods for administering one or more Listeria-based EphA2 vaccines of the invention to treat or ameliorate symptoms in refractory patients. In a certain embodiment, that a cancer is refractory to a therapy means that at least some significant portion of the cancer cells are not killed or their cell division arrested. The determination of whether the cancer cells are refractory can be made either in vivo or in vitro by any method known in the art for assaying the effectiveness of treatment on cancer cells, using the art-accepted meanings of “refractory” in such a context. In various embodiments, a cancer is refractory where the number of cancer cells has not been significantly reduced, or has increased. The invention also encompasses methods for administering one or more Listeria-based EphA2 vaccines to prevent the onset or recurrence of cancer in patients predisposed to having cancer.

In particular embodiments, the Listeria-based EphA2 vaccines of the invention are administered to reverse resistance or reduced sensitivity of cancer cells to certain hormonal, radiation and chemotherapeutic agents thereby resensitizing the cancer cells to one or more of these agents, which can then be administered (or continue to be administered) to treat or manage cancer, including to prevent metastasis. In a specific embodiment, the Listeria-based EphA2 vaccines of the invention are administered to patients with increased levels of the cytokine IL-6, which has been associated with the development of cancer cell resistance to different treatment regimens, such as chemotherapy and hormonal therapy. In another specific embodiment, the Listeria-based EphA2 vaccines of the invention are administered to patients suffering from breast cancer that have a decreased responsiveness or are refractory to tamoxifen treatment. In another specific embodiment, the Listeria-based EphA2 vaccines of the invention are administered to patients with increased levels of the cytokine IL-6, which has been associated with the development of cancer cell resistance to different treatment regimens, such as chemotherapy and hormonal therapy.

In alternate embodiments, the invention provides methods for treating or managing a patients' cancer comprising administering to the patient one or more Listeria-based EphA2 vaccines of the invention in combination with any other therapy or to patients who have proven refractory to other therapies but are no longer on these therapies. In certain embodiments, the patients being treated by the methods of the invention are patients already being treated with chemotherapy, radiation therapy, hormonal therapy, or biological therapy/immunotherapy. Among these patients are refractory patients and those with cancer despite treatment with existing cancer therapies. In other embodiments, the patients have been treated and have no disease activity and one or more Listeria-based EphA2 vaccines of the invention are administered to prevent the recurrence of cancer.

In preferred embodiments, the existing therapy is chemotherapy. In particular embodiments, the existing therapy includes administration of chemotherapies including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc. Among these patients are patients treated with radiation therapy, hormonal therapy and/or biological therapy/immunotherapy. Also among these patients are those who have undergone surgery for the treatment or management of cancer.

Alternatively, the invention also encompasses methods for treating or managing patients undergoing or having undergone radiation therapy. Among these are patients being treated or previously treated with chemotherapy, hormonal therapy and/or biological therapy/immunotherapy. Also among these patients are those who have undergone surgery for the treatment of cancer.

In other embodiments, the invention encompasses methods for treating patients undergoing or having undergone hormonal therapy and/or biological therapy/immunotherapy. Among these are patients being treated or having been treated with chemotherapy and/or radiation therapy. Also among these patients are those who have undergone surgery for the treatment of cancer.

Additionally, the invention also provides methods of treatment or management of cancer as an alternative to chemotherapy, radiation therapy, hormonal therapy, and/or biological therapy/immunotherapy where the therapy has proven or may prove too toxic, i.e., results in unacceptable or unbearable side effects, for the subject being treated. The subject being treated with the methods of the invention may, optionally, be treated with other cancer therapies such as surgery, chemotherapy, radiation therapy, hormonal therapy or biological therapy, depending on which therapy was found to be unacceptable or unbearable.

In other embodiments, the invention provides administration of one or more Listeria-based EphA2 vaccines of the invention without any other cancer therapies for the treatment of cancer, but who have proved refractory to such treatments. In specific embodiments, patients refractory to other cancer therapies are administered one or more EphA2 vaccines in the absence of cancer therapies.

In other embodiments, patients with a pre-cancerous condition associated with cells that overexpress EphA2 can be administered vaccines of the invention to treat the disorder and decrease the likelihood that it will progress to malignant cancer. In a specific embodiments, the pre-cancerous condition is high-grade prostatic intraepithelial neoplasia (PIN), fibroadenoma of the breast, fibrocystic disease, or compound nevi.

In yet other embodiments, the invention provides methods of treating, preventing and/or managing hyperproliferative cell disorders other than cancer, particularly those associated with overexpression of EphA2, including but not limited to, asthma, chromic obstructive pulmonary disorder (COPD), fibrosis (e.g., lung, kidney, heart and liver fibrosis), restenosis (smooth muscle and/or endothelial), psoriasis, etc. These methods include methods analogous to those described above for treating, preventing and managing cancer, for example, by administering the EphA2 vaccines of the invention, combination therapy (see, e.g., Section 5.4.3, infra, for examples of other therapies to administer in combination with the EphA2 vaccines to a subject to treat, prevent or manage a hyperproliferative disorder other than cancer), administration to patients refractory to particular treatments, etc.

5.4.1.2. Cancers

Cancers and related disorders that can be treated, prevented, or managed by methods and compositions of the present invention include but are not limited to cancers of an epithelial cell origin and/or an endothelial origin. Examples of such cancers include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fingating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America)

The methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscarama, and osteosarcoma; and other tumors, including melanoma, xeroderma pigmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the skin, lung, colon, breast, prostate, bladder, kidney, pancreas, ovary, or uterus. In other specific embodiments, sarcoma, melanoma, or leukemia is treated or prevented.

In some embodiments, the cancer is malignant and overexpresses EphA2. In other embodiments, the disorder to be treated is a pre-cancerous condition associated with cells that overexpress EphA2. In a specific embodiments, the pre-cancerous condition is high-grade prostatic intraepithelial neoplasia (PIN), fibroadenoma of the breast, fibrocystic disease, or compound nevi.

In preferred embodiments, the methods and compositions of the invention are used for the treatment and/or prevention of breast, ovarian, esophageal, colon, ovarian, lung, and prostate cancers and melanoma and are provided below by example rather than by limitation.

In another preferred embodiment, the methods and compositions of the invention are used for the treatment and/or prevention of cancers of T cell origin, including, but not limited to, leukemias and lymphomas.

5.4.1.3. Treatment of Breast Cancer

In specific embodiments, patients with breast cancer are administered an effective amount of one or more Listeria-based EphA2 vaccines of the invention. In another embodiment, the peptides of the invention can be administered in combination with an effective amount of one or more other agents useful for breast cancer therapy including but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), her-2 antibodies, e.g., herceptin, tamoxifen, the combination of tamoxifen and cytotoxic chemotherapy, taxanes (such as docetaxel and paclitaxel). In a further embodiment, peptides of the invention can be administered with taxanes plus standard doxorubicin and cyclophosphamide for adjuvant treatment of node-positive, localized breast cancer.

In a specific embodiment, patients with pre-cancerous fibroadenoma of the breast or fibrocystic disease are administered a Listeria-based EphA2 vaccine of the invention to treat the disorder and decrease the likelihood that it will progress to malignant breast cancer. In another specific embodiment, patients refractory to treatment, particularly hormonal therapy, more particularly tamoxifen therapy, are administered a Listeria-based EphA2 vaccine of the invention to treat the cancer and/or render the patient non-refractory or responsive.

5.4.1.4. Treatment of Colon Cancer

In specific embodiments, patients with colon cancer are administered an effective amount of one or more Listeria-based EphA2 vaccines of the invention. In another embodiment, the peptides of the invention can be administered in combination with an effective amount of one or more other agents useful for colon cancer therapy including but not limited to: AVASTIN™ (bevacizumab), the combination of 5-FU and leucovorin, the combination of 5-FU and levamisole, irinotecan (CPT-11) or the combination of irinotecan, 5-FU and leucovorin (IFL).

5.4.1.5. Treatment of Prostate Cancer

In specific embodiments, patients with prostate cancer are administered an effective amount of one or more Listeria-based EphA2 vaccines of the invention. In another embodiment, the peptides of the invention can be administered in combination with an effective amount of one or more other agents useful for prostate cancer therapy including but not limited to: external-beam radiation therapy, interstitial implantation of radioisotopes (i.e., I¹²⁵, palladium, iridium), leuprolide or other LHRH agonists, non-steroidal antiandrogens (flutamide, nilutamide, bicalutamide), steroidal antiandrogens (cyproterone acetate), the combination of leuprolide and flutamide, estrogens such as DES, chlorotrianisene, ethinyl estradiol, conjugated estrogens U.S.P., DES-diphosphate, radioisotopes, such as strontium-89, the combination of external-beam radiation therapy and strontium-89, second-line hormonal therapies such as aminoglutethimide, hydrocortisone, flutamide withdrawal, progesterone, and ketoconazole, low-dose prednisone, or other chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel.

In a specific embodiment, patients with pre-cancerous high-grade prostatic intraepithelial neoplasia (PIN) are administered an EphA2 vaccine of the invention to treat the disorder and decrease the likelihood that it will progress to malignant prostate cancer.

5.4.1.6. Treatment of Melanoma

In specific embodiments, patients with melanoma are administered an effective amount of one or more Listeria-based EphA2 vaccines of the invention. In another embodiment, the peptides of the invention can be administered in combination with an effective amount of one or more other agents useful for melanoma cancer therapy including but not limited to: dacarbazine (DTIC), nitrosoureas such as carmustine (BCNU) and lomustine (CCNU), agents with modest single agent activity including vinca alkaloids, platinum compounds, and taxanes, the Dartmouth regimen (cisplatin, BCNU, and DTIC), interferon alpha (IFN-α), and interleukin-2 (IL-2). In a specific embodiment, an effective amount of one or more EphA2 vaccines of the invention can be administered in combination with isolated hyperthermic limb perfusion (ILP) with melphalan (L-PAM), with or without tumor necrosis factor-alpha (TNF-α) to patients with multiple brain metastases, bone metastases, and spinal cord compression to achieve symptom relief and some shrinkage of the tumor with radiation therapy.

In a specific embodiment, patients with pre-cancerous compound nevi are administered a Listeria-based EphA2 vaccine of the invention to treat the disorder and decrease the likelihood that it will progress to malignant melanoma.

5.4.1.7. Treatment of Ovarian Cancer

In specific embodiments, patients with ovarian cancer are administered an effective amount of one or more Listeria-based EphA2 vaccines of the invention. In another embodiment, the peptides of the invention can be administered in combination with an effective amount of one or more other agents useful for ovarian cancer therapy including but not limited to: intraperitoneal radiation therapy, such as p³² therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. It is contemplated that an effective amount of one or more Listeria-based EphA2 vaccines of the invention is administered in combination with the administration Taxol for patients with platinum-refractory disease. Included is the treatment of patients with refractory ovarian cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors.

5.4.1.8. Treatment of Lung Cancers

In specific embodiments, patients with small lung cell cancer are administered an effective amount of one or more Listeria-based EphA2 vaccines of the invention. In another embodiment, the peptides of the invention can be administered in combination with an effective amount of one or more other agents useful for lung cancer therapy including but not limited to: thoracic radiation therapy, cisplatin, vincristine, doxorubicin, and etoposide, alone or in combination, the combination of cyclophosphamide, doxorubicin, vincristine/etoposide, and cisplatin (CAV/EP), local palliation with endobronchial laser therapy, endobronchial stents, and/or brachytherapy.

In other specific embodiments, patients with non-small lung cell cancer are administered an effective amount of one or more Listeria-based EphA2 vaccines of the invention in combination with an effective amount of one or more other agents useful for lung cancer therapy including but not limited to: palliative radiation therapy, the combination of cisplatin, vinblastine and mitomycin, the combination of cisplatin and vinorelbine, paclitaxel, docetaxel or gemcitabine, the combination of carboplatin and paclitaxel, interstitial radiation therapy for endobronchial lesions or stereotactic radio surgery.

5.4.1.9. Treatment of T Cell Malignancies

In specific embodiments, patients with T cell malignancies, such as leukemias and lymphomas (see, e.g., section 5.8.1.1), are administered an effective amount of one or more Listeria-based EphA2 vaccines of the invention. In another embodiment, the EphA2 vaccines of the invention can be administered in combination with an effective amount of one or more other agents useful for the prevention, treatment or amelioration of cancer, particularly T cell malignancies or one or more symptoms thereof, said combination therapies comprising administering to a subject in need thereof a prophylactically or therapeutically effective amount of one or more Listeria-based EphA2 vaccines of the invention and a prophylactically or therapeutically effective amount of one or more cancer therapies, including chemotherapies, hormonal therapies, biological therapies, immunotherapies, or radiation therapies.

In another specific embodiment, patients with T cell malignancies are administered an effective amount of one or more Listeria-based EphA2 vaccines of the invention in combination with one or more cancer chemotherapeutic agents, such as but not limited to: doxorubicin, epirubicin, cyclophosphamide, 5-fluorouracil, taxanes such as docetaxel and paclitaxel, leucovorin, levamisole, irinotecan, estramustine, etoposide, vinblastine, dacarbazine, nitrosoureas such as carmustine and lomustine, vinca alkaloids, platinum compounds, cisplatin, mitomycin, vinorelbine, gemcitabine, carboplatin, hexamethylmelamine and/or topotecan. Such methods can optionally further comprise the administration of other cancer therapies, such as but not limited to radiation therapy, biological therapies, hormonal therapies and/or surgery.

In yet another specific embodiment, patients with T cell malignancies are administered an effective amount of one or more Listeria-based EphA2 vaccines of the invention in combination with one or more types of radiation therapy, such as external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. Such methods can optionally further comprise the administration of other cancer therapies, such as but not limited to chemotherapies, biological therapies/immunotherapies, hormonal therapies and/or surgery.

In yet another specific embodiment, patients with T cell malignancies are administered an effective amount of one or more Listeria-based EphA2 vaccines of the invention in combination with one or more biological therapies/immunotherapies or hormonal therapies, such as tamoxifen, leuprolide or other LHRH agonists, non-steroidal antiandrogens (flutamide, nilutamide, bicalutamide), steroidal antiandrogens (cyproterone acetate), estrogens (DES, chlorotrianisene, ethinyl estradiol, congugated estrogens U.S.P., DES-diphosphate), aminoglutethimide, hydrocortisone, flutamide withdrawal, progesterone, ketoconazole, prednisone, interferon-α, interleukin-2, tumor necrosis factor-α, and/or melphalan. Biological therapies also included are cytokines such as but not limited to TNF ligand family members such as TRAIL anti-cancer agonists that induce apoptosis, TRAIL antibodies that bind to TRAIL receptors 1 and 2 otherwise known as DR4 and DR5 (Death Domain Containing Receptors 4 and 5), as well as DR4 and DR5. TRAIL and TRAIL antibodies, ligands and receptors are known in the art and described in U.S. Pat. Nos. 6,342,363, 6,284,236, 6,072,047 and 5,763,223. Such methods can optionally further comprise the administration of other cancer therapies, such as but not limited to radiation therapy, chemotherapies, and/or surgery.

In yet another specific embodiment, patients with T cell malignancies are administered an effective amount of one or more Listeria-based EphA2 vaccines of the invention in combination with standard and experimental therapies of T cell malignancies. Standard and experimental therapies of T cell malignancies that can be used in the methods and compositions of the invention include, but are not limited to, antibody therapy (e.g., Campath®, anti-Tac, HuM291 (humanized murine IgG2 monoclonal antibody against CD3), antibody drug conjugates (e.g., Mylotarg), radiolabeled monoclonal antibodies (e.g., Bexxar, Zevalin, Lym-1)), cytokine therapy, aggressive combination chemotherapy with or without cytotoxic agents, purine analogs, hematopoietic stem cell transplantation, and T cell mediated therapy (e.g., CD8+ T cells with anti-leukemic activity against target antigens including but not limited to leukemia specific proteins (e.g., bcr/abl, PML/RARa, EMV/AML-1), leukemia-associated proteins (e.g., proteinase 3, WT-1, h-TERT, hdm-2)). (See Riddell et el., 2002, Cancer Control, 9(2):114-122; Dearden et al., 2002, Medical Oncology, 19, Suppl. S27-32; Waldmann et al. 2000, Hematology (Am Soc Hematol Educ Program):394-408).

5.4.2. Treatment or Prevention of Disorders Associated with Aberrant Angiogenesis

EphA2 is as a marker of angiogenic blood vessels and plays a critical role in angiogenesis or neovascularization (see, e.g., Ogawa et al., 2000, Oncogene. 19(52):6043-52; Hess et al., 2001, Cancer Res. 61(8):3250-5). Angiogenesis is characterized by the invasion, migration and proliferation of smooth muscle and endothelial cells. The growth of new blood vessels, or angiogenesis, contributes to pathological conditions such as diabetic retinopathy (Adonis et al., 1994, Amer. J. Ophthal., 118:445), rheumatoid arthritis (Peacock et al., 1992, J. Exp. Med., 175:1135) and osteoarthritis (Ondrick et al., 1992, Clin.-Podiatr.-Med.-Surg. 9:185).

The Listeria-based compositions of the invention may therefore be administered to a subject in need thereof to prevent, manage, treat or ameliorate a disorder associated with aberrant angiogenesis or one or more symptoms thereof.

Disorders that are associated with or characterized by aberrant angiogenesis and may be prevented, treated, managed, or ameliorated with the Listeria-based compositions of the invention include, but are not limited to, neoplastic diseases (non-limiting examples are metastases of tumors and leukemia); diseases of ocular neovascularization (non-limiting examples are age-related macular degeneration, diabetic retinopathy, and retinopathy of prematurity, vascular restenosis); skin diseases (non-limiting examples are infantile hemangiomas, verruca vulgaris, psoriasis, basal cell and squamous cell carcinomas, cutaneous melanoma, Kaposi's sarcoma, neurofibromatosis, recessive dystrophic epidermolysis bullosa); arthritis (non-limiting examples are rheumatoid arthritis, ankylosing spondylitis, systemic lupus, psoriatic arthropathy, Reiter's syndrome, and Sjogren's syndrome); gynecologic diseases (non-limiting examples are endometriosis, preeclampsia during pregnancy, carcinoma of the ovary, endometrium and cervix); and cardiovascular diseases (non-limiting examples are formation of atherosclerotic plaques, atherosclerosis and coronary artery disease).

In specific embodiments, the disorders that are associated with or characterized by aberrant angiogenesis and that may be prevented, treated, managed, or ameliorated with the Listeria-based compositions of the invention include chronic articular rheumatism, psoriasis, diabetic retinopathy, neovascular glaucoma, macular degeneration, capillary proliferation in atherosclerotic plaques as well as cancers in which EphA2 is expressed in the vasculature. Such cancer disorders can include, for example, solid tumors, tumor metastasis, angiofibromas, retrolental, fibroplasia, hemangiomas, Kaposi's sarcoma.

In certain embodiments, the Listeria-based compositions are employed in combination therapy regimens involving other therapies. Non-limiting examples of such therapies include analgesics, angiogenesis inhibitors, anti-cancer therapies and anti-inflammatory agents, in particular analgesics and angiogenesis inhibitors.

5.4.2.1. Patient Population

The present invention encompasses methods for treating, managing, or preventing a disorder associated with aberrant angiogenesis or a symptom thereof, in a subject comprising administering one or more Listeria-based EphA2 vaccines. The methods of the invention comprise the administration of one or more Listeria-based EphA2 vaccines to patients suffering from or expected to suffer from (e.g., patients with a genetic predisposition for or patients that have previously suffered from) a disorder associated with aberrant angiogenesis. Such patients may have been previously treated or are currently being treated for the disorder. In accordance with the invention, a Listeria-based EphA2 vaccine may be used as any line of therapy, including, but not limited to, a first, second, third and fourth line of therapy. Further, in accordance with the invention, a Listeria-based EphA2 vaccine can be used before any adverse effects or intolerance of the Listeria-based EphA2 vaccine therapies occurs. The invention encompasses methods for administering one or more Listeria-based EphA2 vaccines of the invention to prevent the onset or recurrence of a disorder associated with aberrant angiogenesis.

In one embodiment, the invention also provides methods of treatment or management of a disorder associated with aberrant angiogenesis as alternatives to current therapies. In a specific embodiment, the current therapy has proven or may prove too toxic (i.e., results in unacceptable or unbearable side effects) for the patient. In another embodiment, the patient has proven refractory to a current therapy. In such embodiments, the invention provides for the administration of one or more Listeria-based EphA2 vaccines of the invention without any other therapies for treating or managing the disorder associated with aberrant angiogenesis. In certain embodiments, one or more Listeria-based EphA2 vaccines of the invention can be administered to a patient in need thereof instead of another therapy to treat or manage a disorder associated with aberrant angiogenesis.

The present invention also encompasses methods for administering one or more Listeria-based EphA2 vaccines of the invention to treat or ameliorate symptoms of a disorder associated with aberrant angiogenesis in patients that are or have become refractory to non-Listeria-based EphA2 vaccine therapies. The determination of whether the symptoms are refractory can be made either in vivo or in vitro by any method known in the art for assaying the effectiveness of a therapy on affected cells in the disorder associated with aberrant angiogenesis, or in patients that are or have become refractory to non-Listeria-based EphA2 vaccine therapies.

5.4.3. Other Therapies

In some embodiments, therapy by administration of one or more Listeria-based EphA2 vaccines is combined with the administration of one or more therapies such as, but not limited to, chemotherapies, radiation therapies, hormonal therapies, and/or biological therapies/immunotherapies. Prophylactic/therapeutic agents include, but are not limited to, proteinaceous molecules, including, but not limited to, peptides, polypeptides, proteins, including post-translationally modified proteins, peptides etc.; or small molecules (less than 1000 daltons), inorganic or organic compounds; or nucleic acid molecules including, but not limited to, double-stranded or single-stranded DNA, or double-stranded or single-stranded RNA, as well as triple helix nucleic acid molecules. Prophylactic/therapeutic agents can be derived from any known organism (including, but not limited to, animals, plants, bacteria, fungi, and protista, or viruses) or from a library of synthetic molecules.

In a specific embodiment, the methods of the invention encompass administration of a Listeria-based EphA2 vaccine of the invention in combination with the administration of one or more prophylactic/therapeutic agents, including antibodies, that are inhibitors of kinases such as, but not limited to, ABL, ACK, AFK, AKT (e.g., AKT-1, AKT-2, and AKT-3), ALK, AMP-PK, ATM, Auroral, Aurora2, bARK1, bArk2, BLK, BMX, BTK, CAK, CaM kinase, CDC2, CDK, CK, COT, CTD, DNA-PK, EGF-R, ErbB-1, ErbB-2, ErbB-3, ErbB-4, ERK (e.g., ERK1, ERK2, ERK3, ERK4, ERK5, ERK6, ERK7), ERT-PK, FAK, FGR (e.g., FGF1R, FGF2R), FLT (e.g., FLT-1, FLT-2, FLT-3, FLT-4), FRK, FYN, GSK (e.g., GSK1, GSK2, GSK3-alpha, GSK3-beta, GSK4, GSK5), G-protein coupled receptor kinases (GRKs), HCK, HER2, HKII, JAK (e.g., JAK1, JAK2, JAK3, JAK4), JNK (e.g., JNK1, JNK2, JNK3), KDR, KIT, IGF-1 receptor, IKK-1, IKK-2, INSR (insulin receptor), IRAK1, IRAK2, IRK, ITK, LCK, LOK, LYN, MAPK, MAPKAPK-1, MAPKAPK-2, MEK, MET, MFPK, MHCK, MLCK, MLK3, NEU, NIK, PDGF receptor alpha, PDGF receptor beta, PHK, PI-3 kinase, PKA, PKB, PKC, PKG, PRK1, PYK2, p38 kinases, p135tyk2, p34cdc2, p42cdc2, p42mapk, p44mpk, RAF, RET, RIP, RIP-2, RK, RON, RS kinase, SRC, SYK, S6K, TAK1, TEC, TIE1, TIE2, TRKA, TXK, TYK2, UL13, VEGF, VEGFR1, VEGFR2, YES, YRK, ZAP-70, and all subtypes of these kinases (see e.g., Hardie and Hanks (1995) The Protein Kinase Facts Book, I and II, Academic Press, San Diego, Calif.). In preferred embodiments, a Listeria-based EphA2 vaccine of the invention is administered in combination with the administration of one or more prophylactic/therapeutic agents that are inhibitors of Eph receptor kinases (e.g., EphA2, EphA4). In a most preferred embodiment, an EphA2 vaccine of the invention is administered in combination with the administration of one or more prophylactic/therapeutic agents that are inhibitors of EphA2.

In a specific embodiment, the methods of the invention encompass administration of a Listeria-based EphA2 vaccine of the invention in combination with the administration of one or more therapeutic antibodies. Examples of therapeutic antibodies that can be used in methods of the invention include but are not limited to AVASTIN® which is an anti-VEGF antibody; antibodies that immunospecifically bind to EphA2 induce signal transduction (i.e., EphA2 agonistic antibodies); antibodies that immunospecifically bind to EphrinA1; HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αᵥβ₃ integrin antibody (Applied Molecular Evolution/MedImmune); Campath 1H/LDP-03 which is a humanized anti CD52 IgG1 antibody (Leukosite); Smart M195 which is a humanized anti-CD33 IgG1 antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-alpha antibody (CAT/BASF); CDP870 is a humanized anti-TNF-alpha Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-alpha IgG4 antibody (Celltech); LDP-02 is a humanized anti-αᵥβ₇antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-beta₂ antibody (Cambridge Ab Tech).

In another specific embodiment, the methods of the invention encompass administration of a Listeria-based EphA2 vaccine of the invention in combination with the administration of one or more prophylactic/therapeutic agents that are angiogenesis inhibitors such as, but not limited to: Angiostatin (plasminogen fragment); antiangiogenic antithrombin III; Angiozyme; ABT-627; Bay 12-9566; Benefin; Bevacizumab (AVASTIN™); BMS-275291; cartilage-derived inhibitor (CDI); CAI; CD59 complement fragment; CEP-7055; Col 3; Combretastatin A-4; Endostatin (collagen XVIII fragment); fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methowyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16 kD fragment; -Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-β); Vasculostatin; Vasostatin (calreticulin fragment); ZD6126; ZD6474; famesyl transferase inhibitors (FTI); and bisphosphonates.

In another specific embodiment, the methods of the invention encompass administration of an EphA2 vaccine of the invention in combination with the administration of one or more prophylactic/therapeutic agents that are anti-cancer agents such as, but not limited to: acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decarbazine, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, soluble EphrinA1, EphrinA1-Fc polypeptides, EphA2-Fc polypeptides, EphA2 antisense, EphrinA1 antisense, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin 2 (including recombinant interleukin 2, or rIL2), interferon alpha-2a, interferon alpha-2b, interferon alpha-n1, interferon alpha-n3, interferon beta-I a, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nitrosoureas, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trastuzumab (HERCEPTIN™), trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride. Other anti-cancer drugs include, but are not limited to: 20-epi-1,25 dihydroxyvitamin D3, 5-ethynyluracil, abiraterone, aclarubicin, acylfulvene, adecypenol, adozelesin, aldesleukin, ALL-TK antagonists, altretamine, ambamustine, amidox, amifostine, aminolevulinic acid, amrubicin, amsacrine, anagrelide, anastrozole, andrographolide, angiogenesis inhibitors, antagonist D, antagonist G, antarelix, anti-dorsalizing morphogenetic protein-1, antiandrogens, antiestrogens, antineoplaston, aphidicolin glycinate, apoptosis gene modulators, apoptosis regulators, apurinic acid, ara-CDP-DL-PTBA, arginine deaminase, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitor, bicalutamide, bisantrene, bisaziridinylspermine, bisnafide, bistratene A, bizelesin, breflate, bropirimine, budotitane, buthionine sulfoximine, calcipotriol, calphostin C, camptothecin derivatives, canarypox IL-2, capecitabine, carboxamide-amino-triazole, carboxyamidotriazole, CaRest M3, CARN 700, cartilage derived inhibitor, carzelesin, casein kinase inhibitors (ICOS), castanospermine, cecropin B, cetrorelix, chloroquinoxaline sulfonamide, cicaprost, cis-porphyrin, cladribine, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, deslorelin, dexamethasone, dexifosfamide, dexrazoxane, dexverapamil, diaziquone, didemnin B, didox, diethylnorspermine, dihydro-5-azacytidine, dihydrotaxol, dioxamycin, diphenyl spiromustine, docetaxel, docosanol, dolasetron, doxifluridine, droloxifene, dronabinol, duocarmycin SA, ebselen, ecomustine, edelfosine, edrecolomab, eflomithine, elemene, emitefur, epirubicin, epristeride, estramustine analogue, estrogen agonists, estrogen antagonists, etanidazole, etoposide phosphate, exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, heregulin, hexamethylene bisacetamide, hypericin, ibandronic acid, idarubicin, idoxifene, idramantone, ilmofosine, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, letrozole, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, losoxantrone, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, menogaril, merbarone, meterelin, methioninase, metoclopramide, MIF inhibitor, mifepristone, miltefosine, mirimostim, mismatched double stranded RNA, mitoguazone, mitolactol, mitomycin analogues, mitonafide, mitotoxin fibroblast growth factor-saporin, mitoxantrone, mofarotene, molgramostim, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, N-substituted benzamides, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, O6-benzylguanine, octreotide, okicenone, oligonucleotides, onapristone, ondansetron, ondansetron, oracin, oral cytokine inducer, ormaplatin, osaterone, oxaliplatin, oxaunomycin, paclitaxel, paclitaxel analogues, paclitaxel derivatives, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, porfimer sodium, porfiromycin, prednisone, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyrazoloacridine, pyridoxylated hemoglobin polyoxyethylene conjugate, raf antagonists, raltitrexed, ramosetron, ras famesyl protein transferase inhibitors, ras inhibitors, ras-GAP inhibitor, retelliptine demethylated, rhenium Re 186 etidronate, rhizoxin, ribozymes, RII retinamide, rogletimide, rohitukine, romurtide, roquinimex, rubiginone B1, ruboxyl, safingol, saintopin, SarCNU, sarcophytol A, sargramostim, Sdi 1 mimetics, semustine, senescence derived inhibitor 1, sense oligonucleotides, signal transduction inhibitors, signal transduction modulators, single chain antigen binding protein, sizofiran, sobuzoxane, sodium borocaptate, sodium phenylacetate, solverol, somatomedin binding protein, sonermin, sparfosic acid, spicamycin D, spiromustine, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, taxol, tazarotene, tecogalan sodium, tegafur, tellurapyrylium, telomerase inhibitors, temoporfin, temozolomide, teniposide, tetrachlorodecaoxide, tetrazomine, thaliblastine, thalidomide, thiocoraline, thioguanine, thrombopoietin, thrombopoietin mimetic, thymalfasin, thymopoietin receptor agonist, thymotrinan, thyroid stimulating hormone, tin ethyl etiopurpurin, tirapazamine, titanocene bichloride, topsentin, toremifene, totipotent stem cell factor, translation inhibitors, tretinoin, triacetyluridine, triciribine, trimetrexate, triptorelin, tropisetron, turosteride, tyrosine kinase inhibitors, tyrphostins, UBC inhibitors, ubenimex, urogenital sinus-derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozole, zanoterone, zeniplatin, zilascorb, and zinostatin stimalamer. Preferred additional anti-cancer drugs are 5-fluorouracil and leucovorin.

In more particular embodiments, the present invention also comprises the administration of one or more Listeria-based EphA2 vaccines of the invention in combination with the administration of one or more therapies such as, but not limited to anti-cancer agents such as those disclosed in Table 5 below, preferably for the treatment of breast, ovary, melanoma, prostate, colon and lung cancers as described above.

TABLE 5

| Therapeutic Agent | Administration | Dose | Intervals |
|---|---|---|---|
| doxorubicin | Intravenous | 60-75 mg/m² on Day 1 | 21 day intervals |
| hydrochloride | | | |
| (Adriamycin RDF ® | | | |
| and Adriamycin | | | |
| PFS ®) | | | |
| epirubicin | Intravenous | 100-120 mg/m² on Day 1 of | 3-4 week cycles |
| hydrochloride | | each cycle or divided equally | |
| (Ellence ™) | | and given on Days 1-8 of the | |
| | | cycle | |
| fluorousacil | Intravenous | How supplied: | |
| | | 5 ml and 10 ml vials | |
| | | (containing 250 and 500 mg | |
| | | flourouracil respectively) | |
| docetaxel | Intravenous | 60-100 mg/m² over 1 hour | Once every 3 weeks |
| (Taxotere ®) | | | |
| paclitaxel | Intravenous | 175 mg/m² over 3 hours | Every 3 weeks for 4 courses |
| (Taxol ®) | | | (administered sequentially to |
| | | | doxorubicin-containing |
| | | | combination chemotherapy) |
| tamoxifen citrate | Oral | 20-40 mg | Daily |
| (Nolvadex ®) | (tablet) | Dosages greater than 20 mg | |
| | | should be given in divided | |
| | | doses (morning and evening) | |
| leucovorin calcium | Intravenous or | How supplied: | Dosage is unclear from text. |
| for injection | intramuscular | 350 mg vial | PDR 3610 |
| | injection | | |
| luprolide acetate | Single | 1 mg (0.2 ml or 20 unit mark) | Once a day |
| (Lupron ®) | subcutaneous | | |
| | injection | | |
| flutamide | Oral (capsule) | 250 mg | 3 times a day at 8 hour |
| (Eulexin ®) | | (capsules contain 125 mg | intervals (total daily dosage |
| | | flutamide each) | 750 mg) |
| nilutamide | Oral | 300 mg or 150 mg | 300 mg once a day for 30 |
| (Nilandron ®) | (tablet) | (tablets contain 50 or 150 mg | days followed by 150 mg |
| | | nilutamide each) | once a day |
| bicalutamide | Oral | 50 mg | Once a day |
| (Casodex ®) | (tablet) | (tablets contain 50 mg | |
| | | bicalutamide each) | |
| progesterone | Injection | USP in sesame oil 50 mg/ml | |
| ketoconazole | Cream | 2% cream applied once or | |
| (Nizoral ®) | | twice daily depending on | |
| | | symptoms | |
| prednisone | Oral | Initial dosage may vary from | |
| | (tablet) | 5 mg to 60 mg per day | |
| | | depending on the specific | |
| | | disease entity being treated. | |
| estramustine | Oral | 14 mg/kg of body weight | Daily given in 3 or 4 divided |
| phosphate sodium | (capsule) | (i.e. one 140 mg capsule for | doses |
| (Emcyt ®) | | each 10 kg or 22 lb of body | |
| | | weight) | |
| etoposide or VP-16 | Intravenous | 5 ml of 20 mg/ml solution | |
| | | (100 mg) | |
| dacarbazine | Intravenous | 2-4.5 mg/knowing | Once a day for 10 days. |
| (DTIC-Dome ®) | | | May be repeated at 4 week |
| | | | intervals |
| polifeprosan 20 with | wafer placed in | 8 wafers, each containing 7.7 mg | |
| carmustine implant | resection cavity | of carmustine, for a total | |
| (BCNU) (nitrosourea) | | of 61.6 mg, if size and shape | |
| (Gliadel ®) | | of resection cavity allows | |
| cisplatin | Injection | How supplied: | |
| | | solution of 1 mg/ml in multidose | |
| | | vials of 50 mL and | |
| | | 100 mL | |
| mitomycin | Injection | supplied in 5 mg and 20 mg | |
| | | vials (containing 5 mg and 20 mg | |
| | | mitomycin) | |
| gemcitabine HCl | Intravenous | For NSCLC-2 schedules | 4 week schedule- |
| (Gemzar ®) | | have been investigated and | Days 1, 8 and 15 of each 28- |
| | | the optimum schedule has not | day cycle. Cisplatin |
| | | been determined | intravenously at 100 mg/m² |
| | | 4 week schedule- | on day 1 after the infusion of |
| | | administration intravenously | Gemzar. |
| | | at 1000 mg/m² over 30 | 3 week schedule- |
| | | minutes on 3 week schedule- | Days 1 and 8 of each 21 day |
| | | Gemzar administered | cycle. Cisplatin at dosage of |
| | | intravenously at 1250 mg/m² | 100 mg/m² administered |
| | | over 30 minutes | intravenously after |
| | | | administration of Gemzar on |
| | | | day 1. |
| carboplatin | Intravenous | Single agent therapy: | Every 4 weeks |
| (Paraplatin ®) | | 360 mg/m² I.V. on day 1 | |
| | | (infusion lasting 15 minutes | |
| | | or longer) | |
| | | Other dosage calculations: | |
| | | Combination therapy with | |
| | | cyclophosphamide, Dose | |
| | | adjustment recommendations, | |
| | | Formula dosing, etc. | |
| Ifosamide | Intravenous | 1.2 g/m² daily | 5 consecutive days |
| (Ifex ®) | | | Repeat every 3 weeks or |
| | | | after recovery from |
| | | | hematologic toxicity |
| Topotecan | Intravenous | 1.5 mg/m² by intravenous | 5 consecutive days, starting |
| hydrochloride | | infusion over 30 minutes | on day 1 of 21 day course |
| (Hycamtin ®) | | daily | |

The invention also encompasses administration of the Listeria-based EphA2 vaccines of the invention in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

In a specific embodiment, the methods of the invention encompass administration of a Listeria-based EphA2 vaccine of the invention in combination with the administration of one or more anti-inflammatory agents. Any anti-inflammatory agent, including agents useful in therapies for inflammatory disorders, well-known to one of skill in the art can be used in the compositions and methods of the invention. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™) metaproterenol (ALUPENT™), pirbuterol (MAXAIR™), terbutlaine (BRETHAIRE™ and BRETHINE™), albuterol (PROVENTIL™, RPETABS™, and VOLMAX™), formoterol (FORADIL AEROLIZER™), and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes (see Table 6, infra, for non-limiting examples of leukotriene and typical dosages of such agents)).

In certain embodiments, the anti-inflammatory agent is an agent useful in the prevention, management, treatment, and/or amelioration of asthma or one or more symptoms thereof. Non-limiting examples of such agents include adrenergic stimulants (e.g., catecholamines (e.g., epinephrine, isoproterenol, and isoetharine), resorcinols (e.g., metaproterenol, terbutaline, and fenoterol), and saligenins (e.g., salbutamol)), adrenocorticoids, blucocorticoids, corticosteroids (e.g., beclomethadonse, budesonide, flunisolide, fluticasone, triamcinolone, methylprednisolone, prednisolone, and prednisone), other steroids, beta2-agonists (e.g., albtuerol, bitolterol, fenoterol, isoetharine, metaproterenol, pirbuterol, salbutamol, terbutaline, formoterol, salmeterol, and albutamol terbutaline), anti-cholinergics (e.g., ipratropium bromide and oxitropium bromide), IL-4 antagonists (including antibodies), IL-5 antagonists (including antibodies), IL-13 antagonists (including antibodies), PDE4-inhibitor, NF-Kappa-β inhibitor, VLA-4 inhibitor, CpG, anti-CD23, selectin antagonists (TBC 1269), mast cell protease inhibitors (e.g., tryptase kinase inhibitors (e.g., GW-45, GW-58, and genisteine), phosphatidylinositide-3′ (PI3)-kinase inhibitors (e.g., calphostin C), and other kinase inhibitors (e.g., staurosporine) (see Temkin et al., 2002 J Immunol 169(5):2662-2669; Vosseller et al., 1997 Mol. Biol. Cell 8(5):909-922; and Nagai et al., 1995 Biochem Biophys Res Commun 208(2):576-581)), a C3 receptor antagonists (including antibodies), immunosuppressant agents (e.g., methotrexate and gold salts), mast cell modulators (e.g., cromolyn sodium (INTAL™) and nedocromil sodium (TILADE™)), and mucolytic agents (e.g., acetylcysteine)). In a specific embodiment, the anti-inflammatory agent is a leukotriene inhibitor (e.g., montelukast (SINGULAIR™), zafirlukast (ACCOLATE™), pranlukast (ONON™), or zileuton (ZYFLO™) (see Table 6)).

TABLE 6
Leukotriene Inhibitors for Asthma Therapy

| Leukotriene Modifier | Usual Daily Dosage |
|---|---|
| Montelukast | 4 mg for 2-5 years old |
| (SINGULAIR ™) | 5 mg for 6 to 15 years old |
| | 10 mg for 15 years and older |
| Zafirlukast | 10 mg b.i.d. for 5 to 12 years old twice daily |
| (ACCOLATE ™) | 20 mg b.i.d. for 12 years or older twice daily |
| Pranlukast (ONON ™) | Only avialable in Asia |
| Zyleuton (ZYFLO ™) | 600 mg four times a day for 12 years and |
| | older |

TABLE 7
H₁ Antihistamines

| Chemical class and | |
|---|---|
| representative drugs | Usual daily dosage |
| Ethanolamine | |
| Diphehydramine | 25-50 mg every 4-6 hours |
| Clemastine | 0.34-2.68 mg every 12 hours |
| Ethylenediamine | |
| Tripelennamine | 25-50 mg every 4-6 hours |
| Alkylamine | |
| Brompheniramine | 4 mg every 4-6 hours; or 8-12 mg of SR |
| | form every 8-12 hour |
| Chlorpheniramine | 4 mg every 4-6 hours; or 8-12 mg of SR |
| | form every 8-12 hour |
| Triprolidine (1.25 mg/5 ml) | 2.5 mg every 4-6 hours |
| Phenothiazine | |
| Promethazine | 25 mg at bedtime |
| Piperazine | |
| Hydroxyzine | 25 mg every 6-8 hours |
| Piperidines | |
| Astemizole (nonsedating) | 10 mg/day |
| Azatadine | 1-2 mg every 12 hours |
| Cetirzine | 10 mg/day |
| Cyproheptadine | 4 mg every 6-8 hour |
| Fexofenadine (nonsedating) | 60 mg every 12 hours |
| Loratidine (nonsedating) | 10 mg every 24 hours |

Cancer therapies as well as therapies for hyperproliferative cell disorders other than cancer and their dosages, routes of administration and recommended usage are known in the art and have been described in such literature as the Physician's Desk Reference (56th ed., 2002, 57th ed., 2003, and 58th ed., 2004).

### 5.5. Biological Activity

Toxicity and efficacy of the prophylactic and/or therapeutic protocols of the instant invention can be determined by standard pharmaceutical procedures in experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the vaccine or test compound that achieves a half-maximal inhibition of symptoms) as determined in animal studies. Such information can be used to more accurately determine useful doses in humans. Levels in plasma may be measured, for example, by high performance liquid chromatography.

The anti-cancer activity of the therapies used in accordance with the present invention also can be determined by using various experimental animal models for the study of cancer, such as an immunocompetent mouse model, e.g., Balb/c or C57/B1/6, or transgenic mice where a mouse EphA2 is replaced with the human EphA2, mouse models to which murine tumor cell lines engineered to express human EphA2 are administered, animal models described in Section 6 infra, or any animal model (including hamsters, rabbits, etc.) known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

Compounds for use in therapy can be tested in other suitable animal model systems prior to testing in humans, including but not limited to in rats, mice, chicken, cows, monkeys, rabbits, hamsters, etc., for example, the animal models described above. The compounds can then be used in the appropriate clinical trials.

Further, any assays known to those skilled in the art can be used to evaluate the prophylactic and/or therapeutic utility of the combinatorial therapies disclosed herein for treatment or prevention of cancer.

### 5.6. Vaccine Compositions

The compositions of the invention include bulk drug compositions useful in the manufacture of non-pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of one or more Listeria-based EphA2 vaccines of the invention. The Listeria-based EphA2 vaccines of the invention may comprise one or more EphA2 antigenic peptide-expressing Listeria and a pharmaceutically acceptable carrier.

In a specific embodiment, a composition of the invention comprises a Listeria-based EphA2 vaccine and an additional prophylactic or therapeutic, e.g., anti-cancer, agent. In accordance with this embodiment, the composition may further comprise a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete) or, more preferably, MF59C.1 adjuvant available from Chiron, Emeryville, Calif.), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer a Listeria-based EphA2 vaccine of the invention or the combination of a Listeria-based EphA2 vaccine of the invention and a prophylactic agent or therapeutic agent useful for preventing or treating cancer, e.g., encapsulation in liposomes, microparticles, microcapsules, recombinant cells capable of expressing the EphA2 antigenic peptide, receptor-mediated endocytosis (see, e.g., Wu and Wu, 1987, J. Biol. Chem. 262:4429-4432), construction of a nucleic acid as part of a retroviral or other vector, etc. Methods of administering a Listeria-based EphA2 vaccine or the combination of a Listeria-based EphA2 vaccine of the invention and prophylactic or therapeutic agent, but are not limited to, parenteral administration (e.g., intradermal, intramuscular, intraperitoneal, intravenous and subcutaneous), epidural, and mucosal (e.g., intranasal, inhaled, and oral routes). In a specific embodiment, a Listeria-based EphA2 vaccine of the invention or the combination of a Listeria-based EphA2 vaccine of the invention and prophylactic or therapeutic agent are administered intramuscularly, intravenously, or subcutaneously. The Listeria-based EphA2 vaccine of the invention or the combination of a Listeria-based EphA2 vaccine of the invention and prophylactic or therapeutic agent may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the Listeria-based EphA2 vaccine of the invention or the combination of a Listeria-based EphA2 vaccine of the invention and prophylactic or therapeutic agents of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

In yet another embodiment, the Listeria-based EphA2 vaccine of the invention or the combination of a Listeria-based EphA2 vaccine of the invention and prophylactic or therapeutic agent can be delivered in a controlled release or sustained release system. In one embodiment, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the EphA2 antigenic peptide-expressing Listeria of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; International Publication Nos. WO 99/15154 and WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

5.6.1. Formulations

Pharmaceutical compositions for use in accordance with the present invention may be formulated in conventional manner using one or more physiologically acceptable carriers or excipients.

Thus, the EphA2 antigenic peptide-expressing Listeria of the invention and their physiologically acceptable salts and solvates be formulated for administration by inhalation or insufflation (either through the mouth or the nose) or oral, parenteral or mucosal (such as buccal, vaginal, rectal, sublingual) administration. In a preferred embodiment, local or systemic parenteral administration is used.

For oral administration, the Listeria-based EphA2 vaccine may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the prophylactic or therapeutic agents for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The Listeria-based EphA2 vaccine may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The vaccines of the invention may also be formulated in rectal compositions such as suppositories or retention enemas, e.g., containing conventional suppository bases such as cocoa butter or other glycerides.

In addition to the formulations described previously, the prophylactic or therapeutic agents may also be formulated as a depot preparation. Such long acting formulations may be administered by implantation (for example subcutaneously or intramuscularly) or by intramuscular injection. Thus, for example, the prophylactic or therapeutic agents may be formulated with suitable polymeric or hydrophobic materials (for example as an emulsion in an acceptable oil) or ion exchange resins, or as sparingly soluble derivatives, for example, as a sparingly soluble salt.

The invention also provides that a Listeria-based EphA2 vaccine of the invention is packaged in a hermetically sealed container such as an ampoule or sachette indicating the quantity. In one embodiment, the vaccine is supplied as a dry sterilized lyophilized powder or water free concentrate in a hermetically sealed container and can be reconstituted, e.g., with water or saline to the appropriate concentration for administration to a subject.

In a preferred embodiment of the invention, the formulation and administration of various chemotherapeutic, biological/immunotherapeutic and hormonal therapeutic agents for use in combination with the vaccine of the invention are known in the art and often described in the Physician's Desk Reference, (56^{th ed.} 2002). For instance, in certain specific embodiments of the invention, the agents can be formulated and supplied as provided in Table 3.

In other embodiments of the invention, radiation therapy agents such as radioactive isotopes can be given orally as liquids in capsules or as a drink. Radioactive isotopes can also be formulated for intravenous injections. The skilled oncologist can determine the preferred formulation and route of administration.

In certain embodiments, the EphA2 antigenic peptide-expressing Listeria of the invention are formulated at 1 mg/ml, 5 mg/ml, 10 mg/ml, and 25 mg/ml for intravenous injections and at 5 mg/ml, 10 mg/ml, and 80 mg/ml for repeated subcutaneous administration and intramuscular injection. In other embodiments, the EphA2 antigenic peptide-expressing Listeria of the invention are formulated at amounts ranging between approximately 1×10² CFU/ml to approximately 1×10¹² CFU/ml, for example at 1×10² CFU/ml, 5×10² CFU/ml, 1×10³ CFU/ml, 5×10³ CFU/ml, 1×10⁴ CFU/ml, 5×10⁴ CFU/ml, 1×10⁵ CFU/ml, 5×10⁵ CFU/ml, 1×10⁶ CFU/ml, 5×10⁶ CFU/ml, 1×10⁷ CFU/ml, 5×10⁷ CFU/ml, 1×10⁸ CFU/ml, 5×10⁸ CFU/ml, 1×10⁹ CFU/ml, 5×10⁹ CFU/ml, 1×10¹⁰ CFU/ml, 5×10¹⁰ CFU/ml, 1×10¹¹ CFU/ml, 5×10¹¹ CFU/ml, or 1×10¹² CFU/ml.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

5.6.2. Dosages

The amount of the composition of the invention which will be effective in the treatment, prevention or management of cancer can be determined by standard research techniques. For example, the dosage of the Listeria-based EphA2 vaccine of the invention which will be effective in the treatment, prevention or management of cancer can be determined by administering the composition to an animal model such as, e.g., the animal models disclosed herein or known to those skilled in the art. In addition, in vitro assays may optionally be employed to help identify optimal dosage ranges.

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the cancer, and should be decided according to the judgment of the practitioner and each patient's circumstances. Effective doses may be extrapolated from dose-response curves derived from in vitro or animal model test systems.

With respect to the dosage of Listeria in the Listeria-based EphA2 vaccines of the invention, the dosage is based on the amount colony forming units (c.f.u.). Generally, in various embodiments, the dosage ranges are from about 1.0 c.f.u./kg to about 1×10¹⁰ c.f.u./kg; from about 1.0 c.f.u./kg to about 1×10⁸ c.f.u./kg; from about 1×10² c.f.u./kg to about 1×10⁸ c.f.u./kg; and from about 1×10⁴ c.f.u./kg to about 1×10⁸ c.f.u./kg. Effective doses may be extrapolated from dose-response curves derived animal model test systems. In certain exemplary embodiments, the dosage ranges are 0.001-fold to 10,000-fold of the murine LD₅₀, 0.01-fold to 1,000-fold of the murine LD₅₀, 0.1-fold to 500-fold of the murine LD₅₀, 0.5-fold to 250-fold of the murine LD₅₀, 1-fold to 100-fold of the murine LD₅₀, and 5-fold to 50-fold of the murine LD₅₀. In certain specific embodiments, the dosage ranges are 0.00.1-fold, 0.01-fold, 0.1-fold, 0.5-fold, 1-fold, 5-fold, 10-fold, 50-fold, 100-fold, 200-fold, 500-fold, 1,000-fold, 5,000-fold or 10,000-fold of the murine LD₅₀.

For other cancer therapeutic agents administered to a patient, the typical doses of various cancer therapeutics known in the art are provided in Table 3. Given the invention, certain preferred embodiments will encompass the administration of lower dosages in combination treatment regimens than dosages recommended for the administration of single agents.

The invention provides for any method of administrating lower doses of known prophylactic or therapeutic agents than previously thought to be effective for the prevention, treatment, management or amelioration of cancer. Preferably, lower doses of known anti-cancer therapies are administered in combination with lower doses of Listeria-based EphA2 vaccines of the invention.

### 5.7. Kits

The invention provides a pack or kit comprising one or more containers filled with a Listeria-based EphA2 vaccine of the invention or a component of a Listeria-based EphA2 vaccine of the invention. Additionally, one or more other prophylactic or therapeutic agents useful for the treatment of a cancer or other hyperproliferative disorder can also be included in the pack or kit. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises one or more a Listeria-based EphA2 vaccines of the invention. In another embodiment, a kit further comprises one or more other prophylactic or therapeutic agents useful for the treatment of cancer or another hyperproliferative disorder, in one or more containers. In other embodiments, the prophylactic or therapeutic agent is a biological or hormonal therapeutic.

### 6. EXAMPLES

### Listeria-Based EphA2 Vaccines Provide Therapeutic and Prophylactic Benefits Against EphA2-Expressing Cancers

The receptor tyrosine kinase EphA2 is selectively over-expressed in a variety of malignant cell types and tumors. Additionally, recent studies have identified patient-derived T lymphocytes that recognize EphA2. As such, EphA2 provides a much-needed target for active immunotherapy. Here, we show that ectopic expression of human EphA2 in the Gram-positive facultative intracellular bacterium Listeria monocytogenes (Listeria) can provide antigen-specific anti-tumor responses in vaccinated animals. Listeria infects critical antigen presenting cells and thereby provides efficacy as a cancer therapy based its ability to induce potent and robust CD4+ and CD8+ T cell responses against encoded antigens. Attenuated Listeria mutant strains, which retain the antigen delivery potency of wild-type bacteria, yet are nearly 10,000-fold less pathogenic in mice, were employed. To demonstrate the efficacy of a Listeria-based EphA2 vaccine, Listeria actA⁻ strains were engineered to express the extracellular (ECD) or intracellular (ICD) domain of human EphA2 (actA-hEphA2-ECD or actA-hEphA2-ICD). Expression and secretion of hEphA2-EX and —CO from Listeria was confirmed by Western blot analysis. Protective immunization with actA-hEphA2EX significantly inhibited the subcutaneous growth of CT26 cells that express full-length hEphA2 (p=0.0037). As controls, mice vaccinated with the parental actA strain developed tumors that were comparable to vehicle-treated control mice. Protective immunization with actA-hEphA2CO significantly increased the survival rate in mice challenged with RenCA-hEphA2. Subsequently, the therapeutic efficacy of actA-hEphA2-ECD or actA-hEphA2-ICD was evaluated using the experimental CT26-hEphA2 lung tumor model. Following intravenous implantation of tumor cells, Balb/c mice were immunized with actA, actA-hEphA2EX or actA-hEphA2-ICD. Immunization with either actA-hEphA2-ECD or actA-hEphA2-ICD significantly prolonged survival (median survival >43 days, p=0.0035), as compared to matched controls (vehicle or actA median survival time was 19 and 20 days, respectively). Importantly, 80% of the huEphA2 immunized mice survived until Day 43 following tumor implantation. Together, these data demonstrate that Listeria-mediated vaccination targeting the EphA2 tumor antigen can provide both preventative and therapeutic efficacy against a variety of malignancies.

### 6.1. Example 1

### Listeria Life Cycle

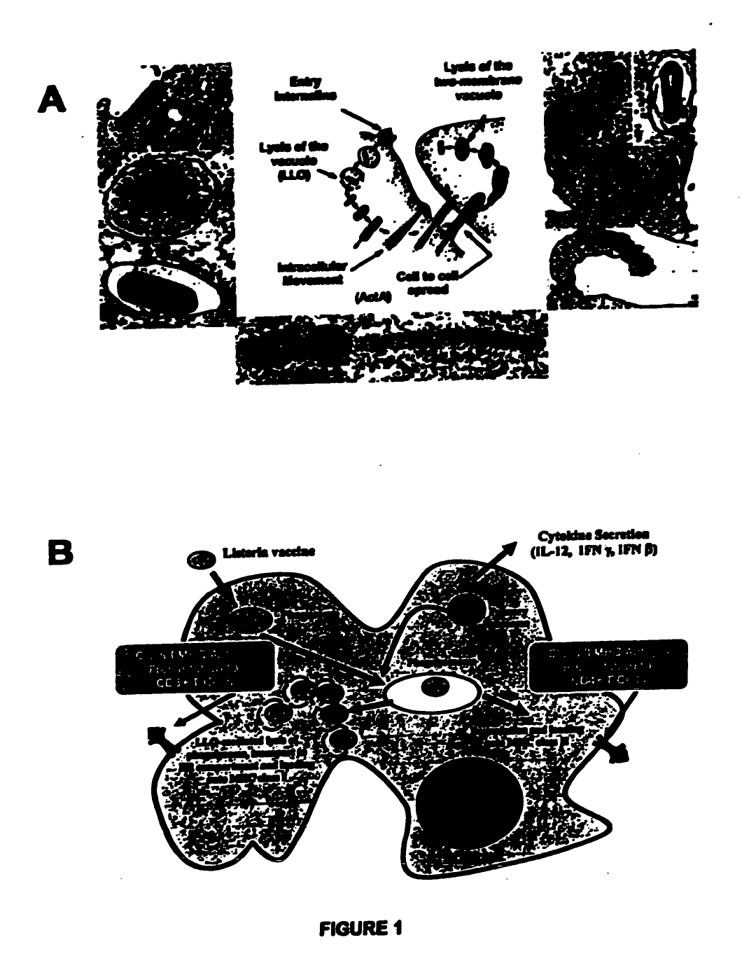

The life cycle of Listeria monocytogenes, encompassing the steps of endocytosis, phagolysosomal lysis, and cell to cell spread, are shown in FIG. 1A-1B.

### 6.2. Example 2

### Construction of EphA2-Expressing and Control Listeria Strains

6.2.1. Background

Given the mechanisms by which Listeria programs the presentation of heterologous antigens via the MHC class I pathway, the efficiency of both expression of heterologous genes and secretion of the newly synthesized protein from the bacterium into the cytoplasm of the infected (antigen presenting) cell is related directly to the potency of CD8+ T cell priming and/or activation. As the level of Ag-specific T cell priming is related directly to vaccine efficacy, the efficiency of heterologous protein expression and secretion is linked directly to vaccine potency. Thus, the efficiency of EphA2 expression and secretion was optimized to maximize the potency of Listeria-based vaccines, in terms of priming and/or activating CD8+ T cell responses specific for the encoded EphA2 protein.

6.2.2. Preparation of Mutant Listeria Strains.

Listeria strains were derived from 10403S (Bishop et al., J. Immunol. 139:2005 (1987)). Listeria strains with in-frame deletions of the indicated genes were generated by SOE-PCR and allelic exchange with established methods (Camilli et al., Mol. Microbiol. 8:143 (1993)). The mutant strain LLO L461T (DP-L4017) was described in Glomski, et al., J. Cell. Biol. 156: 1029 (2002), incorporated by reference herein. The actA⁻ mutant (DP-L4029) is the DP-L3078 strain described in Skoble et al., J. of Cell Biology, 150: 527-537 (2000), incorporated by reference herein in its entirety, which has been cured of its prophage. (Prophage curing is described in (Lauer et al., J. Bacteriol. 184:4177 (2002)); U.S. patent Publication No. 2003/0203472.)

In some vaccines, mutant strains of Listeria that are deficient with respect to internalin B (Genbank accession number AL591975 (Listeria monocytogenes strain EGD, complete genome, segment 3/12; inlB gene region: nts. 97008-98963), incorporated by reference herein in its entirety, and/or the sequence listed as Genbank accession number NC_{—}003210 (Listeria monocytogenes strain EGD, complete genome, inlB gene region: nts. 457008-458963), incorporated by reference herein in its entirety) are used. One particular actA⁻inlB⁻ strain (DP-L4029inlB) was deposited with the American Type Culture Collection (ATCC) on Oct. 3, 2003, and designated with accession number PTA-5562).

6.2.3. Cloning Vectors

Selected heterologous antigen expression cassette molecular constructs were inserted into pPL2 (Lauer et. al. J. Bacteriol. 2002), or pAM401 (Wirth et. al., J. Bacteriol. 165:831-836), modified to contain the multiple cloning sequence of pPL2 (Aat II smal1 fragment, 171 bps), inserted between blunted Xba I and Nru I recognition sites, within the tetracycline resistance gene (pAM401-MCS). In general, the hly promoter and (selected) signal peptide sequence was inserted between the unique Kpn I and Bam HI sites in the pPL2 or pAM401-MCS plasmid vectors. Selected EphA2 genes (sometimes modified to contain N-terminal and C-terminal epitope tags; see description below) were cloned subsequently into these constructs between unique Bam HI and Sac I sites. Molecular constructs based on the pAM401-MCS plasmid vector were introduced by electroporation into selected Listeria monocytogenes strains also treated with lysozyme, utilizing methods common to those skilled in the art. The expected plasmid structure in Listeria-transfectants was verified by isolating DNA from colonies that formed on chloramphenicol-containing BHI agar plates (10 μg/ml) by restriction enzyme analysis. Recombinant Listeria transformed with various pAM401-MCS based heterologous protein expression cassette constructs were utilized to measure heterologous protein expression and secretion, as described below.

The pPL2 based heterologous protein expression cassette constructs were incorporated into the tRNAArg gene in the genome of selected Listeria strains, according to the methods as described previously (Lauer et al., 2002, J. Bacteriol. 184:4177-4186). Briefly, the pPL2 heterologous protein expression cassette constructs plasmid was first introduced into the E. coli host strain SM10 (Simon et al., 1983, Bio/Technology 1:784-791) by electroporation or by chemical means. Subsequently, the pPL2-based plasmid was transferred from transformed SM10 to the selected Listeria strains by conjugation. Following incubation on drug-selective BHI agar plates containing 7.5 μg of chloramphenicol per ml and 200 μg of streptomycin per ml as described, selected colonies are purified by passaging 3 times on plates with the same composition. To verify integration of the pPL2 vector at the phage attachment site, individual colonies are picked and screened by PCR using the primer pair of forward primer NC16 (5′-gtcaaaacatacgctcttatc-3′) (SEQ ID NO:47) and reverse primer PL95 (5′-acataatcagtccaaagtagatgc-3′) (SEQ ID NO:48). Selected colonies having the pPL2-based plasmid incorporated into the tRNAArg gene in the genome of selected Listeria strains yielded a diagnostic DNA amplicon of 499 bps.

6.2.4. Promoter

Heterologous protein expression cassettes contained the prfA-dependent hly promoter, which drives the transcription of the gene encoding Listeriolysin O (LLO), and is activated within the microenvironment of the infected cell. Nucleotides 205586-206000 (414 bps) were amplified by PCR from Listeria monocytogenes, strain DP-L4056, using the primer pair shown below. The region amplified includes the hly promoter and also the first 28 amino acids of LLO, comprising the secA1 signal peptide (ibid) and PEST domain. The expected sequence of this region for Listeria monocytogenes, strain EGD can be found in GenBank (Accession number: gi|168020481|ref|NC_{—}003210.1|[16802048]).

| | |
|---|---|
| Primer Pair | |
| Forward (KpnI-LLO nts. 1257-1276): | |
| 5′-CTCTGGTACCTCCTTTGATTAGTATATTC | (SEQ ID NO:49) |
| Reverse (Bam HI-LLO nts. X-x): | |
| 5′-CTCTGGATCCATCCGCGTGTTTCTTTTCG | (SEQ ID NO:50) |

(Restriction endonuclease recognition sites are underlined)

The 422 bp PCR amplicon was cloned into the plasmid vector pCR-XL-TOPO (Invitrogen, Carlsbad, Calif.), according to the manufacturer's specifications. The nucleotide sequences of Listeria-specific bases in the pCR-XL-TOPO-hly promoter plasmid clone was determined. Listeria monocytogenes strain DP-L4056 contained eight nucleotide base changes flanking the prfA box in the hly promoter, as compared to the EGD strain. The hly promoter alignment for the Listeria monocytogenes DP-L4056 and EGD strains is shown in the Figure below (SEQ ID NOs: 68 and 69, respectively).

Listeria hly DP--L4056 and EGD Alignment

| Query: | Listeria EGD | |
|---|---|---|
| Subject: | DP-L4056 (wild-type, Portnoy strain) | |
| prfA Box | | |
| Query: 1 | ggtacctcctttgattagtatattcctatcttaaagtgacttttatgttgaggcattaac | 60 |
| | \|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\| \|\|\|\|\|\|\|\|\|\|\| | |
| Sbjct: 1 | ggtacctcctttgattagtatattcctatcttaaagttacttttatgtggaggcattaac | 60 |
| Query: 61 | atttgttaacgacgataaagggacagcaggactagaataaagctataaagcaagcatata | 120 |
| | \|\|\|\|\|\|\|\|\| \|\|\|\| \|\|\| \|\|\| \|\|\|\| \|\|\|\|\|\|\|\|\|\|\|\|\|\|\|\|\|\|\|\|\|\|\|\|\|\|\|\|\|\|\| | |
| | atttgttaatgacgtcaaaaggatagcaagactagaataaagctataaagcaagcatata | |
| Query: 121 | atattgcgtttcatctttagaagcgaatttcgccaatattataattatcaaaagagaggg | 180 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| | atattgcgtttcatctttagaagcgaatttcgccaatattataattatcaaaagagaggg | 180 |
| Shine-Delgarno LLO start | | |
| Query: 181 | gtggcaaacggtatttggcattattaggttaaaaaatgtagaaggagagtgaaacccatg | 240 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| Sbjct: 181 | gtggcaaacggtatttggcattattaggttaaaaaatgtagaaggagagtgaaacccatg | 240 |

The 422 bp DNA corresponding to the hly promoter and secA1 LLO signal peptide were liberated from the pCR-XL-TOPO-hly promoter plasmid clone by digestion with Kpn I and Bam HI, and cloned into the pPL2 plasmid vector (Lauer et al., 2002, J. Bact.), according to conventional methods well-known to those skilled in the art. This plasmid is known as pPL2-hlyP (native).

6.2.5. Cloning and Insertion of EphA2 into pPL2 Vectors for Expression in Selected Recombinant Listeria monocytogenes Strains

The external (EX2) and cytoplasmic (CO) domains of EphA2 which flank the EphA2 transmembrane helix were cloned separately for insertion into various pPL2-signal peptide expression constructs. Genes corresponding to the native mammalian sequence or codon-optimized for expression in Listeria monocytogenes of EphA2 EX2 and CO domains were used. The optimal codons in Listeria (see table, ibid) for each of the 20 amino acids were utilized for codon-optimized EphA2 EX2 and EphA2 CO. The codon-optimized EphA2 EX2 and CO domains were synthesized by extension of overlapping oligonucleotides, using techniques common to those skilled in the art. The expected sequence of all synthesized EphA2 constructs was verified by nucleotide sequencing.

SEQ ID NOS:23, 21 and 22 represent the primary amino acid sequences, together with the native and codon-optimized nucleotide sequences, respectively, for the EX2 domain of EphA2.

SEQ ID NOS: 34, 32 and 33 represent the primary amino acid sequences, together with the native and codon-optimized nucleotide sequences, respectivley, for the CO domain of EphA2.

Additonally, FLAG (Stratagene, La Jolla, Calif.) and myc epitope tags were inserted, respectively, in-frame at the amino and carboxy termini of synthesized EphA2 EX2 and CO genes for detection of expressed and secreted EphA2 by Western blot analysis using antibodies specific for the FLAG or proteins. Thus, the expressed protein had the following ordered elements: NH₂-Signal Peptide-FLAG-EphA2-myc-CO₂. Shown below are the FLAG and myc epitope tag amino acid and codon-optimized nucleotide sequences.

| | |
|---|---|
| FLAG | |
| 5′-GATTATAAAGATGATGATGATAAA | (SEQ ID NO:51) |
| NH₂-DYKDDDDK-CO₂ | (SEQ ID NO:52) |
| Myc | |
| 5′-GAACAAAAATTAATTAGTGAAGAAGATTTA | (SEQ ID NO:53) |
| NH₂-EQKLISEEDL-CO₂ | (SEQ ID NO:54) |

6.2.6. Detection of Synthesized and Secreted Heterologous Proteins by Western Blot Analysis

Synthesis of EphA2 protein and secretion from various selected recombinant Listeria-EphA2 strains was determined by Western blot analysis of trichloroacetic acid (TCA) precipitated bacterial culture fluids. Briefly, mid-log phase cultures of Listeria grown in BHI media were collected in a 50 mL conical centrifuge tube, the bacteria were pelleted, and ice-cold TCA was added to a final [6%] concentration to the bacterial culture supernatant and incubated on ice minimally for 90 min or overnight. The TCA-precipitated proteins were collected by centrifugation at 2400×g for 20 min at 4° C. The pellet was then resuspended in 300-600 μl volume of TE, pH 8.0 containing 15 μg/ml phenol red. Sample dissolution was facilitated by vortexing. Sample pH was adjusted by NH₄OH addition if necessary until color was pink. All samples were prepared for electrophoresis by addition of 100 μl of 4× SDS loading buffer and incubating for 10 min. at 90° C. The samples were then centrifuged from 5 min at 14,000 rpm in a micro-centrifuge, and the supernatants collected and stored at −20° C. For Western bolt analysis, 20 μl of prepared fractions (the equivalent of culture fluids from of 1-4×10⁹ bacteria), were loaded on the 4-12% SDS-PAGE gel, electrophoresed, and the proteins were transferred to PDDF membrane, according to common methods used by those skilled in the art. Transferred membranes were prepared s for incubation with antibody, by incubating in 5% dry milk in PBS for 2 hr. at room temperature with agitation. Antibodies were used under the following dilutions in PBST buffer (0.1% Tween 20 in PBS): (1) Rabbit anti-Myc polyclonal antibody (ICL laboratories, Newberg, Oreg.) at 1:10,000; (2) murine anti-FLAG monoclonal antibody (Stratagene, ibid) at 1:2,000; and, (3) Rabbit anti-EphA2 (carboxy terminus-specific) polyclonal antibody (sc-924, Santa Cruz Biotechnology, Inc., Santa Cruz, Calif.). Specific binding of antibody to protein targets was evaluated by secondary incubation with goat anti-rabbit or anti-mouse antibody conjugated with horseradish peroxidase and detection with the ECL chemilumenescence assay kit (Amersham), and exposure to film.

6.2.7. Secretion of EphA2 Protein by Recombinant Listeria Encoding Various Forms of EphA2

6.2.7.1. Listeria: [Strains DP-L4029 (actA) or DP-L4017 (LLO L461T)]

Expression cassette construct: LLOss-PEST-CO-EphA2 (SEQ ID NO:35)

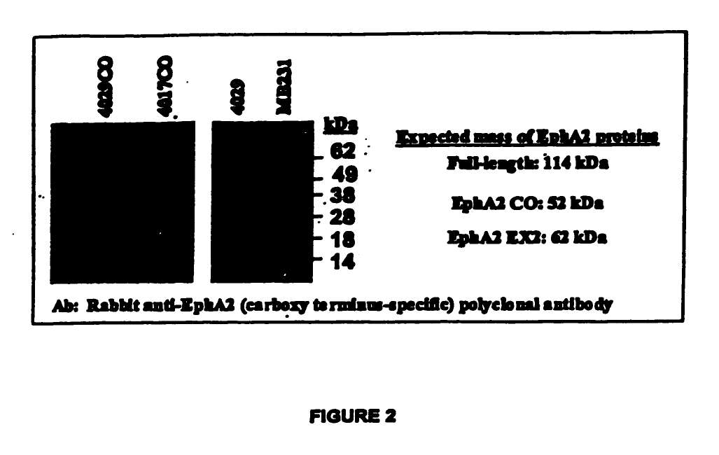

The native sequence of the EphA2 CO domain was genetically fused to the native secA1 LLO sequence, and the heterologous antigen expression cassette under control of the Listeria hly promoter was inserted into the pPL2 plasmid between the Kpn I and Sac I sites as described (ibid). The pPL2-EphA2 plasmid constructs were introduced by conjugation into the Listeria strains DP-L4029 (actA) and DP-L4017 (L461T LLO) as described (ibid). FIG. 2 shows the results of a Western blot analysis of TCA-precipitated bacterial culture fluids of 4029-EphA2 CO and 4017-EphA2 CO. This analysis demonstrated that recombinant Listeria engineered to contain a heterologous protein expression cassette comprised of native sequences corresponding to the secA1 and EphA2 CO fusion protein secreted multiple EphA2-specific fragments that were lower than the 52 kDa expected molecular weight, demonstrating the need for modification of the expression cassette.

6.2.7.2. Listeria: [DP-L4029 (actA)]

Expression cassette constructs:

(SEQ ID NO:26)

Native LLOss-PEST-FLAG-EX2_EphA2-myc-CodonOp

(SEQ ID NO:28)

(CodonOp) LLOss-PEST-(CodonOp)FLAG-EX2_EphA2-myc

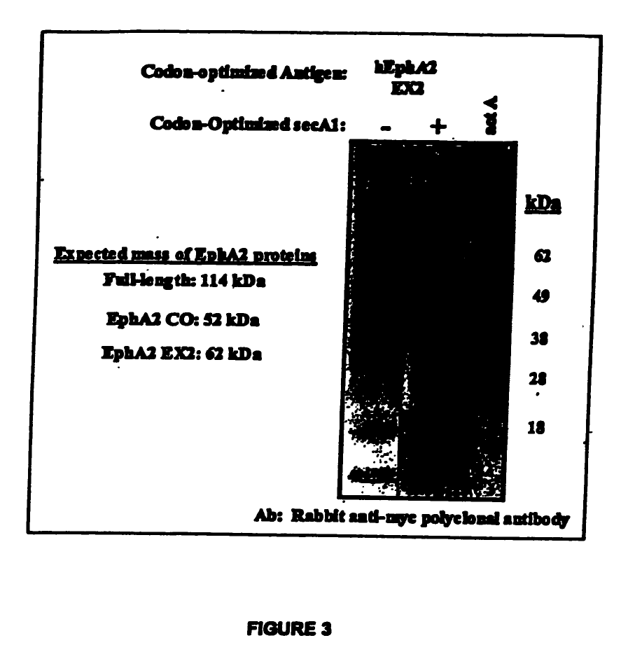

The native secA1 LLO signal peptide sequence or secA1 LLO signal peptide sequence codon-optimized for expression in Listeria was fused genetically with the EphA2 EX2 domain sequence codon-optimized for expression in Listeria , and the heterologous antigen expression cassette under control of the Listeria hly promoter was inserted into the pPL2 plasmid between the Kpn I and Sac I sites as described (ibid). The pPL2-EphA2 plasmid constructs were introduced by conjugation into the Listeria strain DP-L4029 (actA) as described (ibid). FIG. 3 shows the results of a Western blot analysis of TCA-precipitated bacterial culture fluids of Listeria actA encoding either the native or codon-optimized secA1 LLO signal peptide fused with the codon-optimized EphA2 EX2 domain. This analysis demonstrated that the combination of utilizing sequence for both signal peptide and heterologous protein optimized for the preferred codon usage in Listeria monocytogenes resulted in expression of the expected full-length EphA2 EX2 domain protein. Expression of full-length EphA2 EX2 domain protein was poor with codon-optimization of the EphA2 coding sequence alone. The level of heterologous protein expression (fragmented or full-length) was highest when utilizing the Listeria monocytogenes LLO secA1 signal peptide, codon-optimized for expression in Listeria monocytogenes.

6.2.7.3. Listeria: [DP-L4029 (actA)]

Expression cassette constructs:

(SEQ ID NO:37)

Native LLOss-PEST-(CodonOp) FLAG-EphA2_CO-myc

(SEQ ID NO:39)

CodonOp LLOss-PEST-(CodonOp) FLAG-EphA2_CO-myc

(SEQ ID NO:41)

CodonOp PhoD-(CodonOp) FLAG-EphA2_CO-myc

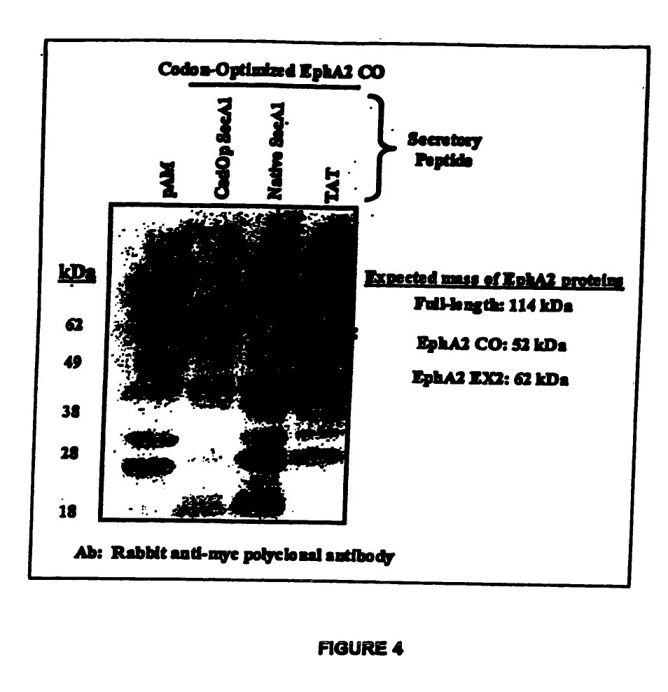

The native secA1 LLO signal peptide sequence or the secA1 LLO signal peptide sequence codon-optimized for expression in Listeria, or, alternatively, the Tat signal peptide of the phoD gene from Bacillus subtilis codon-optimized for expression in Listeria, was fused genetically with the EphA2 CO domain sequence codon-optimized for expression in Listeria, and the heterologous antigen expression cassette under control of the Listeria hly promoter was inserted into the pAM401 -MCS plasmid between the Kpn I and Sac I sites as described (ibid). The pAM401-EphA2 plasmid constructs were introduced by electroporation into the Listeria strain DP-L4029 (actA) as described (ibid). FIG. 4 shows the results of a Western blot analysis of TCA-precipitated bacterial culture fluids of Listeria actA encoding either the native or codon-optimized secA1 LLO signal peptide, or codon-optimized Bacillus subtilis phoD Tat signal peptide fused with the codon-optimized EphA2 CO domain. This analysis demonstrated once again that the combination of utilizing sequence for both signal peptide and heterologous protein optimized for the preferred codon usage in Listeria monocytogenes resulted in expression of the expected full-length EphA2 CO domain protein. Furthermore, expression and secretion of the expected full-length EphA2 CO domain protein resulted from recombinant Listeria encoding codon-optimized Bacillus subtilis phoD Tat signal peptide fused with the codon-optimized EphA2 CO domain. This result demonstrates the novel and unexpected finding that signal peptides from distinct bacterial species can be utilized to program the secretion of heterologous proteins from recombinant Listeria. Expression of full-length EphA2 CO domain protein was poor with codon-optimization of just the EphA2 sequence. The level of heterologous protein expression was highest when utilizing signal peptides codon-optimized for expression in Listeria monocytogenes.

6.2.8. Construction of Listeria Strains Expressing AH1/OVA or AH1-A5/OVA

Mutant Listeria strains expressing a truncated form of a model antigen ovalbumin (OVA), the immunodominant epitope from mouse colorectal cancer (CT26) known as AH1 (SPSYVYHQF) (SEQ ID NO:55), and the altered epitope AH1-A5 (SPSYAYHQF (SEQ ID NO:56); Slansky et al., 2000, Immunity, 13:529-538) were prepared. The pPL2 integrational vector (Lauer et al., J. Bacteriol. 184:4177 (2002); U.S. patent Publication No. 2003/0203472) was used to derive OVA and AH1-A5/OVA recombinant Listeria strains containing a single copy integrated into an innocuous site of the Listeria genome.

6.2.9. Construction of OVA-Expressing Listeria (DP-L4056)

An antigen expression cassette consisting of hemolysin-deleted LLO fused with truncated OVA and contained in the pPL2 integration vector (pPL2/LLO-OVA) is first prepared. The Listeria-OVA vaccine strain is derived by introducing pPL2/LLO-OVA into the phage-cured L. monocytogenes strain DP-L4056 at the PSA (Phage from ScottA) attachment site tRNA^{Arg}-attBB′.

PCR is used to amplify the hemolysin-deleted LLO using the following template and primers:

Source: DP-L4056 genomic DNA

Primers:

Forward (KpnI-LLO nts. 1257-1276):

(SEQ ID NO:57)

5′-CTCTGGTACCTCCTTTGATTAGTATATTC
(Tₘ: LLO-spec: 52° C. Overall: 80° C.)
Reverse (BamHI-XhoI-LLO nts. 2811-2792):

(SEQ ID NO:58)

5′-CAATGGATCCCTCGAGATCATAATTTACTTCATCCC
(Tₘ: LLO-spec: 52° C. Overall: 102° C.)

PCR is also used to amplify the truncated OVA using the following template and primers:

Source: pDP3616 plasmid DNA from DP-E3616 E. coli (Higgins et al., Mol. Molbiol. 31:1631-1641 (1999)).

Primers:

Forward (XhoI-NcoI OVA cDNA nts. 174-186):

(SEQ ID NO:59)

5′-ATTTCTCGAGTCCATGGGGGGTTCTCATCATC
(Tₘ: OVA-spec: 60° C. Overall: 88° C.)
Reverse (XhoI-NotI-HindIII):

(SEQ ID NO:60)

5′-GGTGCTCGAGTGCGGCCGCAAGCTT
(Tₘ: Overall: 82° C.)

One protocol for completing the construction process involves first cutting the LLO amplicon with KpnI and BamHI and inserting the KpnI/BamHI vector into the pPL2 vector (pPL2-LLO). The OVA amplicon is then cut with XhoI and NotI and inserted into the pPL2-LLO which has been cut with XhoI/NotI. (Note: The pPL2 vector does not contain any XhoI sites; pDP-3616 contains one XhoI site, that is exploited in the OVA reverse primer design.) The construct pPL2/LLO-OVA is verified by restriction analysis (KpnI-LLO-XhoI-OVA-NotI) and sequencing. The plasmid pPL2/LLO-OVA is introduced into E. coli by transformation, followed by introduction and integration into Listeria (DP-L4056) by conjugation, exactly as described by Lauer et al. (or into another desired strain of Listeria).

6.2.10. Construction of Listeria Strains Expressing AH1/OVA or AH1-A5/OVA

To prepare Listeria expressing either the AH1/OVA or the AH1-A5/OVA antigen sequences, inserts bearing the antigen are first prepared from oligonucleotides and then ligated into the vector pPL2-LLO-OVA (prepared as described above).

The following oligonucleotides are used in preparation of the AH1 or AH1-A5 insert:

AH1 epitope insert (ClaI-Pst1 compatible ends):

Top strand oligo (AH1 Top):

(SEQ ID NO:61)

5′-CGATTCCCCTAGTTATGTTTACCACCAATTTGCTGCA
Bottom strand oligo (AH1 Bottom):

(SEQ ID NO:62)

5′-GCAAATTGGTGGTAAACATAACTAGGGGAAT

AH1-A5 epitope insert (ClaI-AvaII compatible ends):

The sequence of the AH1-A5 epitope is SPSYAYHQF (SEQ ID NO:56) (5′-AGT CCA AGT Tat GCA Tat CAT CAA TTT-3′) (SEQ ID NO:63).

Top:

(SEQ ID NO:64)

5′-CGATAGTCCAAGTTATGCATATCATCAATTTGC
Bottom:

(SEQ ID NO:65)

5′-GTCGAAATTGATGATATGCATAACTTGGACTAT

The oligonucletide pair for a given epitope are mixed together at an equimolar ratio, heated at 95° C. for 5 min. The oligonucleotide mixture is then allowed to slowly cool. The annealed oligonucleotide pairs are then ligated at a 200 to 1 molar ratio with pPL2-LLO/OVA plasmid prepared by digestion with the relevant restriction enzymes. The identity of the new construct can be verified by restriction analysis and/or sequencing.

The plasmid can then be introduced into E. coli by transformation, followed by introduction and integration into Listeria (DP-L4056) by conjugation, exactly as described by Lauer et al., or into another desired strain of Listeria, such as an actA⁻ mutant strain (DP-L0429), LLO L461T strain (DP-L4017), or actA⁻/inlB⁻ strain (DP-L4029inlB).

### 6.3. Example 3

### Generation of Murine Tumor Cell Lines That Express Human EphA2

6.3.1. Background

A mouse immunotherapy model was created for testing the Listeria-based vaccines of the invention. Three murine tumor cell lines, the CT26 murine colon carcinoma cell line, the B16F10 murine melanoma cell line, and the RenCa murine renal cell carcinoma cell line were created to express high levels of the huEphA2 protein. FACS cell sorting assays were performed to identify CT26, B16F10, and RenCa tumor cells expressing high levels of huEphA2, which were pooled and analyzed by Western blot analysis. Clones were further pooled by FACS cell sorting to generate subclones expressing the highest levels of huEphA2.

6.3.2. Selection of CT26 Murine Colon Carcinoma Cells Expressing High Levels of huEphA2

6.3.2.1. Transfection Assays With Lipofectamine™

CT26 cells were transfected with constructs containing huEphA2 using standard transfection techniques and commercially available Lipofectamine™ according to the manufacturer's instructions.

6.3.2.2. Flow Cytometry (FACS) Analysis

Single cell FACS sorting assays were performed by standard techniques to identify CT26 murine carcinoma tumor cell expressing high levels of human EphA2.

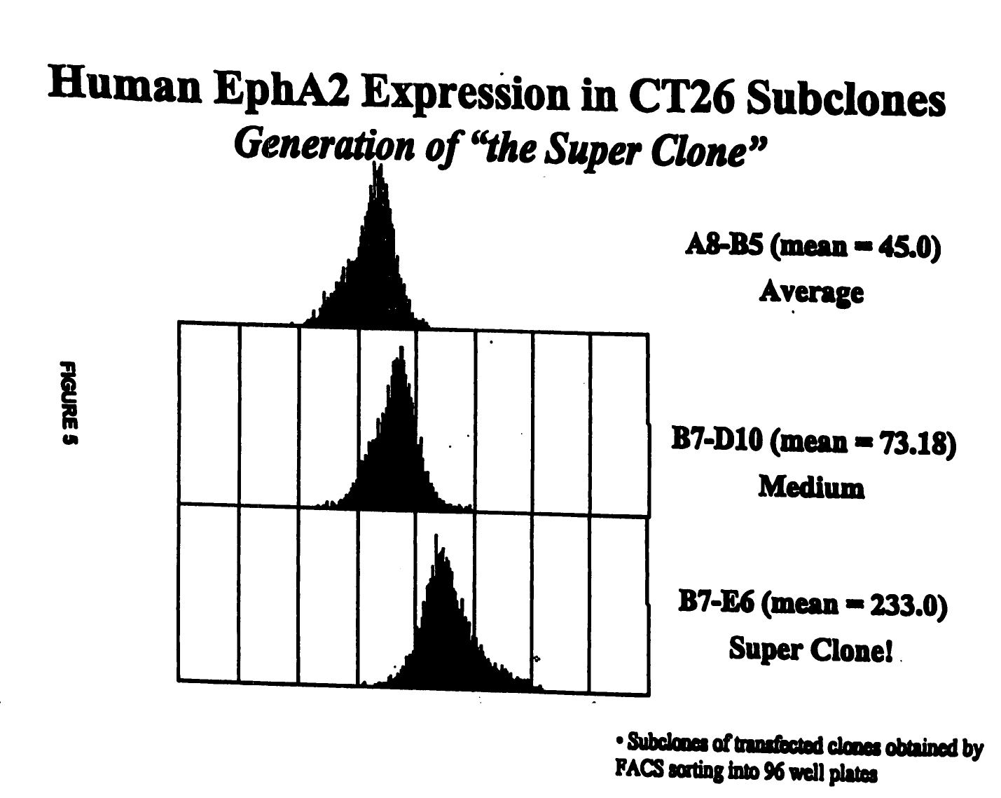

FIG. 5 illustrates a representative experiment, showing that the EphA2-3 clone expressed the highest levels of human EphA2 protein.

6.3.2.3. Western Blot of Pooled Populations Expressing High Levels of huEphA2

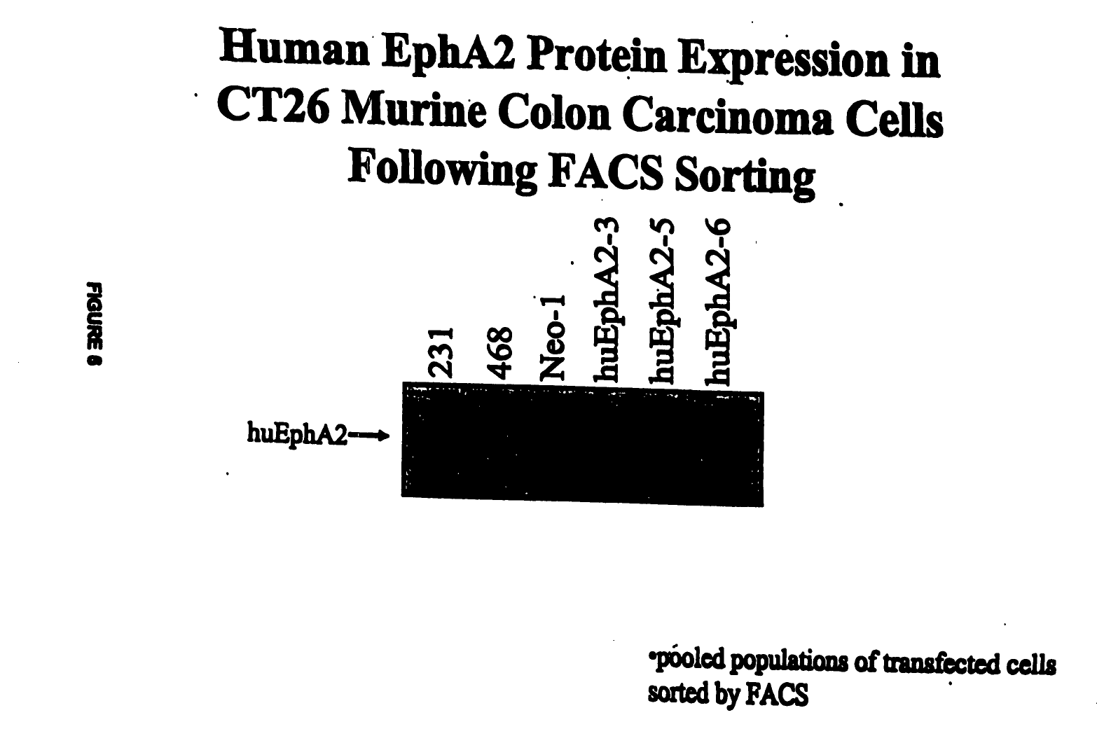

Western blotting was also performed using standard techniques to determine the levels of human EphA2 protein expression in CT26 cells following FACS sorting of pooled populations of cells transfected with various constructs containing the huEphA2 gene. FIG. 6 depicts results of a representative experiment. Compared to various clones tested, the huEphA2-3 clone expressed the highest levels of human EphA2 protein and was selected for the in vivo efficacy studies described below. Cells were further pooled to generate subclones expressing the highest levels of huEphA2.

6.3.3. Selection of B16F10 Murine Melanoma Cells Expressing High Levels of huEphA2

6.3.3.1. Retroviral Transduction

Human EphA2 was introduced into B16F10 murine melanoma cells by a retroviral transduction method to create clones expressing high levels of the protein.

6.3.3.2. Flow Cytometry (FACS) Analysis

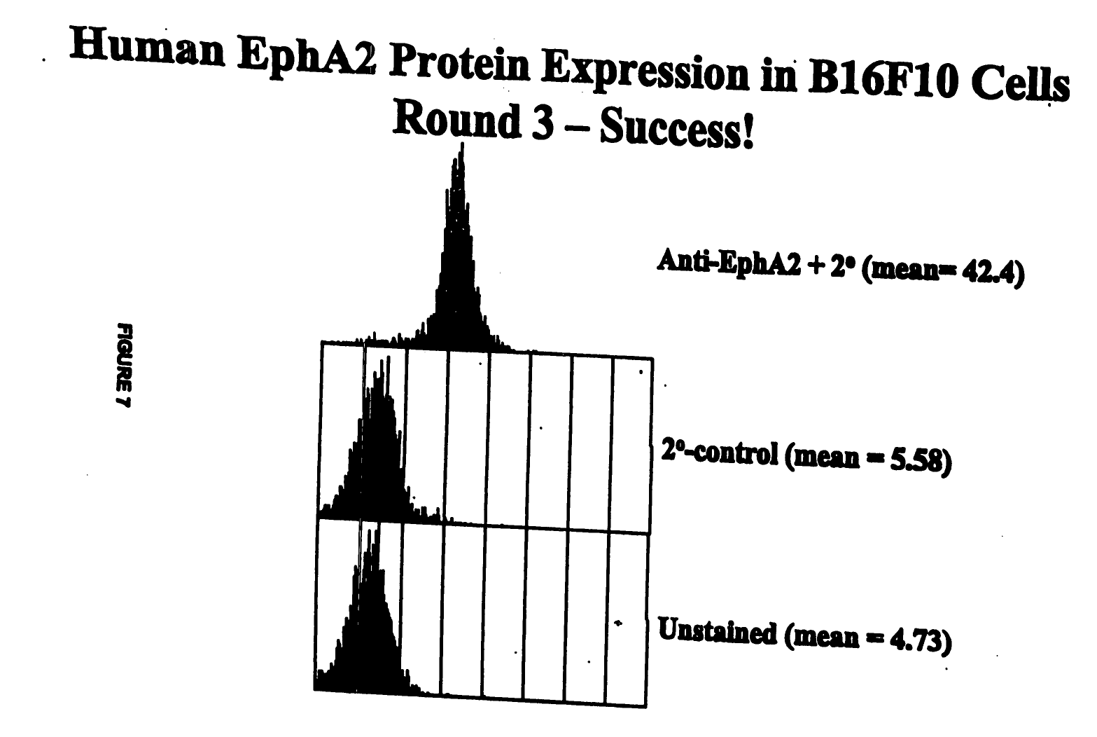

As was performed on the CT26 cells, single cell FACS sorting assays were performed by standard techniques on B16F10 cells expressing huEphA2 to generate clones expressing high levels of huEphA2. Clones expressing the highest levels of huEphA2 were pooled and further examined by Western blot analysis. A representative FACS experiment is depicted in FIG. 7, showing a B16F10 subclone expressing high levels of huEphA2.

6.3.3.3. Western Blot of Pooled Populations Expressing High Levels of huEphA2

Western blotting was also performed as described above to determine levels of huEphA2 protein expression in B16F10 cells following FACS sorting of pooled populations of cells containing the huEphA2 gene introduced by retroviral transduction. Cells were further pooled to generate subclones expressing the highest levels of huEphA2.

6.3.4. Selection of RenCa Murine Renal Cell Carcinoma Cells Expressing High Levels of huEphA2

6.3.4.1. Transfection Assays With Lipofectamine™

RenCa cells were transfected with constructs containing huEphA2 using standard transfection techniques and commercially available Lipofectamine™ according to the manufacturer's instructions.

6.3.4.2. Flow Cytometry (FACS) Analysis

Single cell FACS sorting assays were performed by standard techniques to identify RenCa renal cell carcinoma tumor cells expressing high levels of human EphA2.

6.3.4.3. Western Blot of Pooled Populations Expressing High Levels of huEphA2

Western blotting was also performed using standard techniques to determine the levels of human EphA2 protein expression in RenCa cells following FACS sorting of pooled populations of cells transfected with various constructs containing the huEphA2 gene. Cells were further pooled to generate subclones expressing the highest levels of huEphA2.

6.3.5. Transfection of 293 Cells with pCDNA4 Plasmids Encoding Full-Length EphA2

Expression cassette constructs:
pCDNA4-EphA2

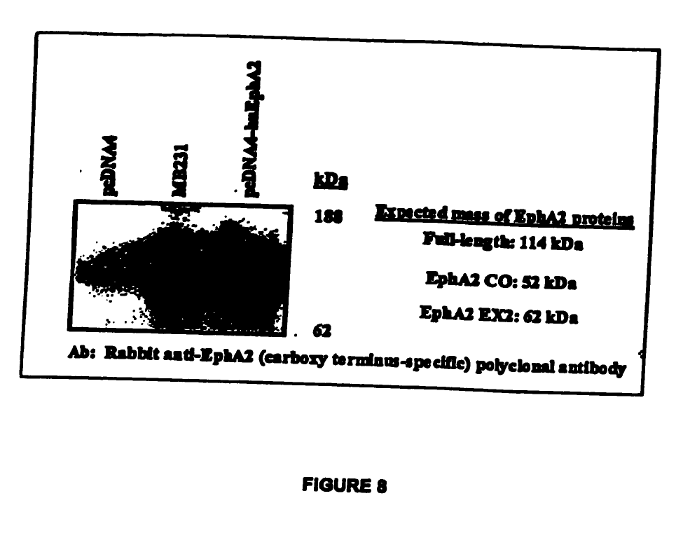

The native full-length EphA2 gene was cloned into the eukaryotic CMV promoter-based expression plasmid pCDNA4 (Invitrogen, Carlsbad, Calif.). FIG. 8 shows the results of a Western blot analysis of lystates prepared from 293 cells transfected with the pCDNA4-EphA2 plasmid, and demonstrates the abundant expression in mammalian cells of full-length EphA2 protein.

### 6.4. Example 4

### Assessment of Antigen-Specific Immune Responses After Vaccination

The vaccines of the present invention can be assessed using a variety of in vitro and in vivo methods. Some assays involve the analysis of antigen-specific T cells from the spleens of mice that have been vaccinated. For example C57Bl/6 or Balb/c are vaccinated by intravenous injection of 0.1 LD₅₀ of a Listeria strain expressing OVA (or other appropriate antigen). Seven days after the vaccination, the spleen cells of the mice are harvested (typically 3 mice per group) by placing the spleens into ice cooled RPMI 1640 medium and preparing a single cell suspension from this. As an alternative, the lymph nodes of the mice could be similarly harvested, prepared as a single cell suspension and substituted for the spleen cells in the assays described below. Typically, spleen cells are assessed for intraveneous or intraperitoneal administration of the vaccine while spleen cells and cells from lymph nodes are assessed for intramuscular, subcutaneous or intradermal administration of the vaccine.

Unless otherwise noted, all antibodies used in these examples can be obtained from Pharmingen, San Diego, Calif.

6.4.1. ELISPOT Assay:

Using a Listeria strain having an OVA antigen as an example, the quantitative frequency of antigen-specific T cells generated upon immunization in a mouse model is assessed using an ELISPOT assay. The antigen-specific T cells evaluated are OVA specific CD8+ or LLO specific CD8+ or CD4+ T cells. This OVA antigen model assesses the immune response to a heterologous tumor antigen inserted into the vaccine and could be substituted with any antigen of interest. The LLO antigen is specific to Listeria. The specific T cells are assessed by detection of cytokine release (e.g. IFN-γ) upon recognition of the specific antigen. PVDF-based 96 well plates (BD Biosciences, San Jose, Calif.) are coated overnight at 4° C. with an anti-murine IFN-γ monoclonal antibody (mAb R4; 5 μg/ml). The plates are washed and blocked for 2 hours at room temperature with 200 μL of complete RPMI. Spleen cells from vaccinated mice (or non vaccinated control mice) are added at 2×10⁵ cells per well and incubated for 20 to 22 hours at 37° C. in the presence of various concentrations of peptides ranging from 0.01 to 10 μM. The peptides used for OVA and LLO are either SL8, an MHC class I epitope for OVA, LLO₁₉₀ (NEKYAQAYPNVS, Invitrogen) an MHC class II epitope for listeriolysin O (Listeria antigen), LLO₂₉₆ (VAYGRQVYL), an MHC class I epitope for listeriolysin O, or LLO₉₁ (GYKDGNEYI), an MHC class I epitope for listeriolysin O. LLO₁₉₀ and LLO₂₉₆ are used in a C57Bl/6 model, while LLO₉₁ is used in a Balb/c model. After washing, the plates are incubated with secondary biotinylated antibodies specific for IFN-γ (XMG1.2) diluted in PBS to 0.5 μg/ml. After incubation at room temperature for 2 hours, the plates are washed and incubated for 1 hour at 37° C. with a 1 nm gold goat anti-biotin conjugate (GAB-1; 1:200 dilution; Ted Pella, Redding, Calif.) diluted in PBS containing 1% BSA. After thorough washing, the plates are incubated at room temperature for 2 to 10 minutes with substrate (Silver Enhancing Kit; 30 ml/well; Ted Pella) for spot development. The plates are then rinsed with distilled water to stop the substrate reaction. After the plates have been air-dried, spots in each well are counted using an automated ELISPOT plate reader (CTL, Cleveland, Ohio). The cytokine response is expressed as the number of IFN-γ spot-forming cells (SFCS) per 2×10⁵ spleen cells for either the OVA specific T cells or the Listeria specific T cells.

6.4.2. Intracellular Cytokine Staining Assay (ICS):

In order to further assess the number of antigen-specific CD8+ or CD4+ T cells and correlate the results with those obtained from ELISPOT assays, ICS is performed and the cells evaluated by flow cytometry analysis. Spleen cells from vaccinated and control groups of mice are incubated with SL8 (stimulates OVA specific CD8+ cells) or LLO₁₉₀ (stimulates LLO specific CD4+ cells) for 5 hours in the presence of Brefeldin A (Pharmingen). The Brefeldin A inhibits secretion of the cytokines produced upon stimulation of the T cells. Spleen cells incubated with an irrelevant MHC class I peptide are used as controls. PMA (phorbol-12-myristate-13-acetate, Sigma) 20 ng/ml and ionomycin (Sigma) 2 μg/ml stimulated spleen cells are used as a positive control for IFN-γ and TNF-α intracellular cytokine staining. For detection of cytoplasmic cytokine expression, cells are stained with FITC-anti-CD4 mAb (RM 4-5) and PerCP-anti-CD8 mAb (53-6.7), fixed and permeabilized with Cytofix/CytoPerm solution (Pharmingen), and stained with PE-conjugated anti-TNF-α mAb (MP6-XT22) and APC-conjugated anti-IFN-γ mAb (XMG1.2) for 30 minutes on ice. The percentage of cells expressing intracellular IFN-γ and/or TNF-α was determined by flow cytometry (FACScalibur, Becton Dickinson, Mountain View, Calif.) and data analyzed using CELLQuest software (Becton Dickinson Immunocytometry System). As the fluorescent labels on the various antibodies can all be distinguished by the FACScalibur, the appropriate cells are identified by gating for those CD8+ and CD4+ that are stained with either or both of the anti-IFN-γ or anti-TNF-α.

6.4.3. Cytokine Expression of Stimulated Spleen Cells:

The level of cytokine secretion by the spleen cells of mice can also be assessed for control and vaccinated C57Bl/6 mice. Spleen cells are stimulated for 24 hours with SL8 or LLO₁₉₀. Stimulation with irrelevant peptide HSV-gB² (Invitrogen, SSIEFARL) is used as a control. The supernatants of the stimulated cells are collected and the levels of T helper-1 and T helper 2 cytokines are determined using an ELISA assay (eBiosciences, CO) or a Cytometric Bead Array Kit (Pharmingen).

6.4.4. Assessment of Cytotoxic T cell Activity:

The OVA specific CD8+ T cells can be further evaluated by assessing their cytotoxic activity, either in vitro or directly in C57Bl/6 mouse in vivo. The CD8+ T cells recognize and lyse their respective target cells in an antigen-specific manner. In vitro cytotoxicity is determined using a chromium release assay. Spleen cells of naive and Listeria-OVA (internal) vaccinated mice are stimulated at a 10:1 ratio with either irradiated EG7.OVA cells (EL-4 tumor cell line transfected to express OVA, ATCC, Manassas, Va.) or with 100 nM SL8, in order to expand the OVA specific T cells in the spleen cell population. After 7 days of culture, the cytotoxic activity of the effector cells is determined in a standard 4-hour ⁵¹Cr-release assay using EG7.OVA or SL8 pulsed EL-4 cells (ATCC, Manassas, Va.) as target cells and EL-4 cells alone as negative control. The YAC-1 cell line (ATCC, Manassas, Va.) is used as targets to determine NK cell activity, in order to distinguish the activity due to T cells from that due to NK cells. The percentage of specific cytotoxicity is calculated as 100×(experimental release−spontaneous release)/(maximal release−spontaneous release). Spontaneous release is determined by incubation of target cells without effector cells. Maximal release is determined by lysing cells with 0.1% Triton X-100. Experiments are considered valid for analysis if spontaneous release is <20% of maximal release.

For the assessment of cytotoxic activity of OVA-specific CD8+ T cells in vivo, spleen cells from naive C57Bl/6 mice are split into two equivalent aliquots. Each group is pulsed with a specific peptide, either target (SL8) or control (HSV-gB²), at 0.5 μg/ml for 90 minutes at 37° C. Cells are then washed 3 times in medium, and twice in PBS+0.1% BSA. Cells are resuspended at 1×10⁷ per ml in warm PBS +0.1% BSA (10 ml or less) for labeling with carboxyfluorescein diacetate succinimidyl ester (CFSE, Molecular Probes, Eugene, Oreg.). To the target cell suspension, 1.25 μL of a 5 mM stock of CFSE is added and the sample mixed by vortexing. To the control cell suspension, a ten-fold dilution of the CFSE stock is added and the sample mixed by vortexing. The cells are incubated at 37° C. for 10 minutes. Staining is stopped by addition of a large volume (>40 ml) of ice-cold PBS. The cells are washed twice at room temperature with PBS, then resuspended and counted. Each cell suspension is diluted to 50×10⁶ per ml, and 100 μL of each population is mixed and injected via the tail vein of either naive or vaccinated mice. After 12-24 hours, the spleens are harvested and a total of 5'10⁶ cells are analyzed by flow cytometry. The high (target) and low (control) fluorescent peaks are enumerated, and the ratio of the two is used to establish the percentage of target cell lysis. The in vivo cytotoxicity assay permits the assessment of lytic activity of antigen-specific T cells without the need of in vitro re-stimulation. Furthermore, this assays assesses the T cell function in their native environment.

### 6.5. Example 5

### In Vivo EphA2 Efficacy Studies

6.5.1. Background

Efficacy studies were performed in mice inoculated with CT26 tumor cells expressing the extracellular domain (ED) of human EphA2 in order to characterize the anti-tumor effect of huEphA2. Endpoints measured were tumor volume and percent survival of the mice after tumor inoculation. The routes of inoculation were subcutaneous (s.c.) and intravenous (i.v.). HBSS and Listeria were administered as controls.

6.5.2. Control Vaccinations With AH1-A5-Expressing Listeria

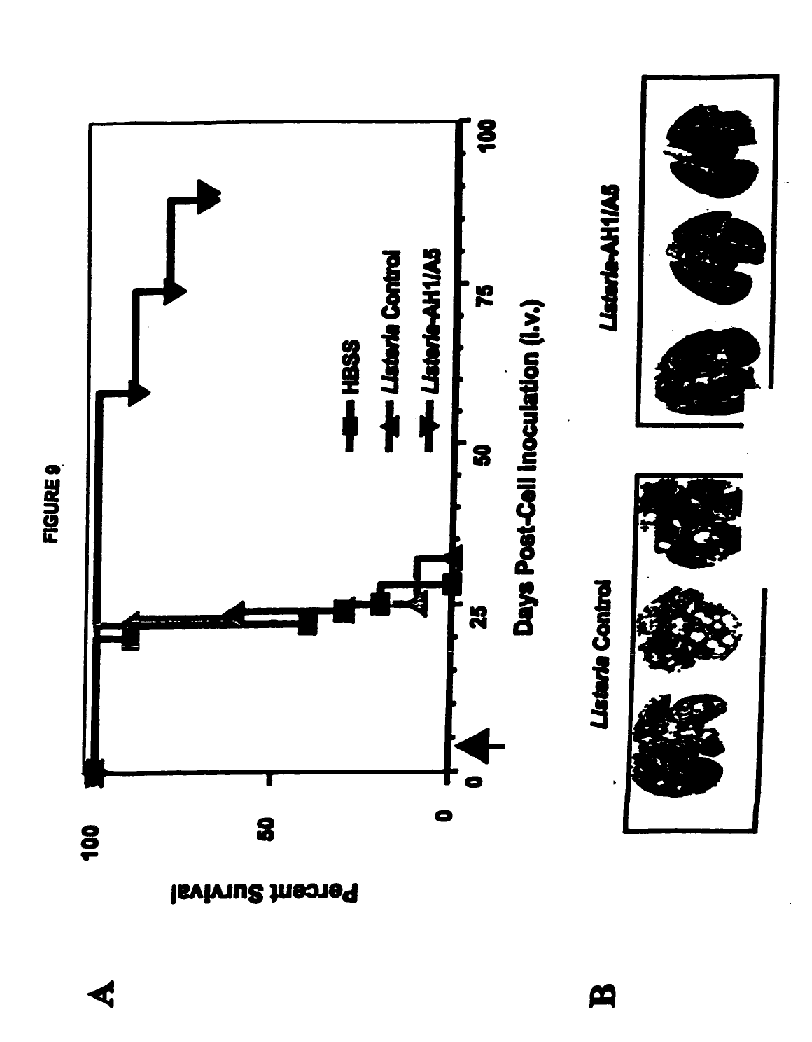

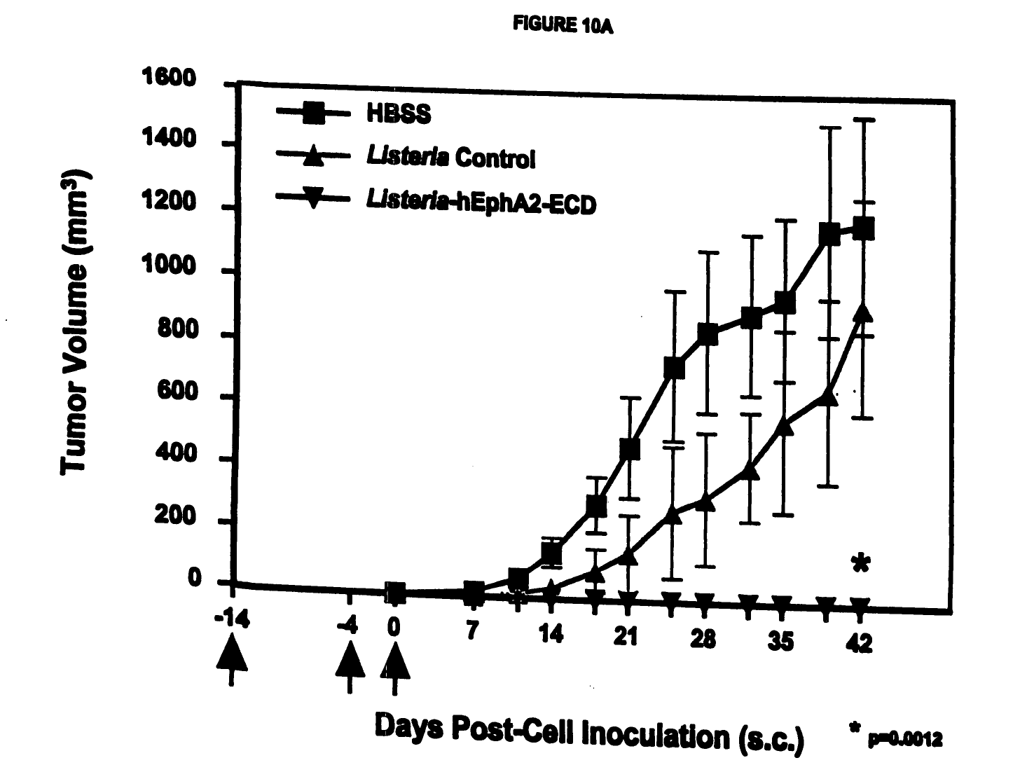

Balb/c mice (n=5) were immunized with 0.1 LD₅₀ Listeria 3 days post-i.v. inoculation of 1×10⁵ CT26 cells. FIG. 9A demonstrates that therapeutic immunization with Listeria expressing AH1-A5 increases survival of the inoculated animals. FIG. 9B shows the result of a separate but otherwise equivalent experiment in which lungs of the experimental mice were harvested on Day 19 following cell inoculation and fixed. Gross assessment of lung nodules was also performed, demonstrating the absence of tumors in the lungs of test animals receiving Listeria-AH1/A5 as compared to control animals receiving a Listeria control.

6.5.3. Prophylactic EphA2 Vaccinations

6.5.3.1. Effect of Immunization with Listeria Expressing ECD of huEphA2 on CT26-hEphA2 Tumor Growth and Survival

Preventive studies were performed utilizing a pool of CT26 cells expressing huEphA2 generated by the single cell FACS assays described above. Groups of ten Balb/c mice per group were inoculated s.c. and groups of five mice per group were inoculated i.v. with CT26 colon carcinoma cells transfected with human EphA2 (“CT26-hEphA2”). The mice were immunized with 0.1 LD50 Listeria control or Listeria expressing the ECD of hEphA2 in a 200 μl bolus. For the studies entailing s.c. inoculations with CD26, AH1/A5 Listeria were used as a positive control. The immunizations were performed 14 and 4 days prior to CT26-hEphA2 tumor challenge. Tumor volume measurements were obtained twice weekly for the course of the study to determine an anti-tumor effect of the vaccinations.

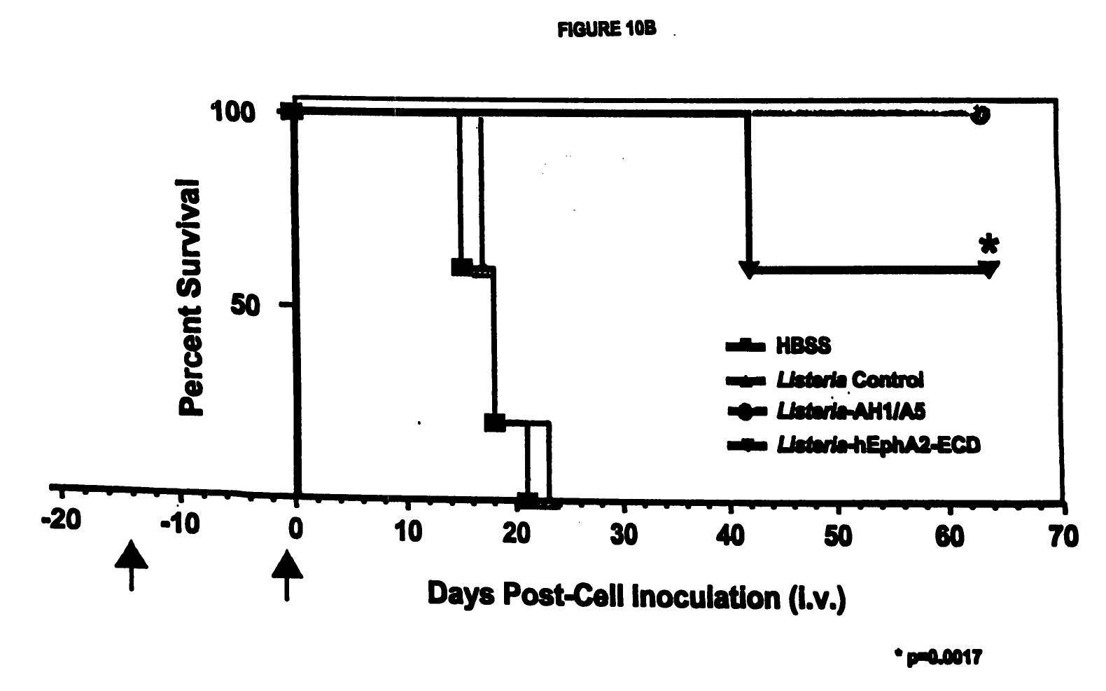

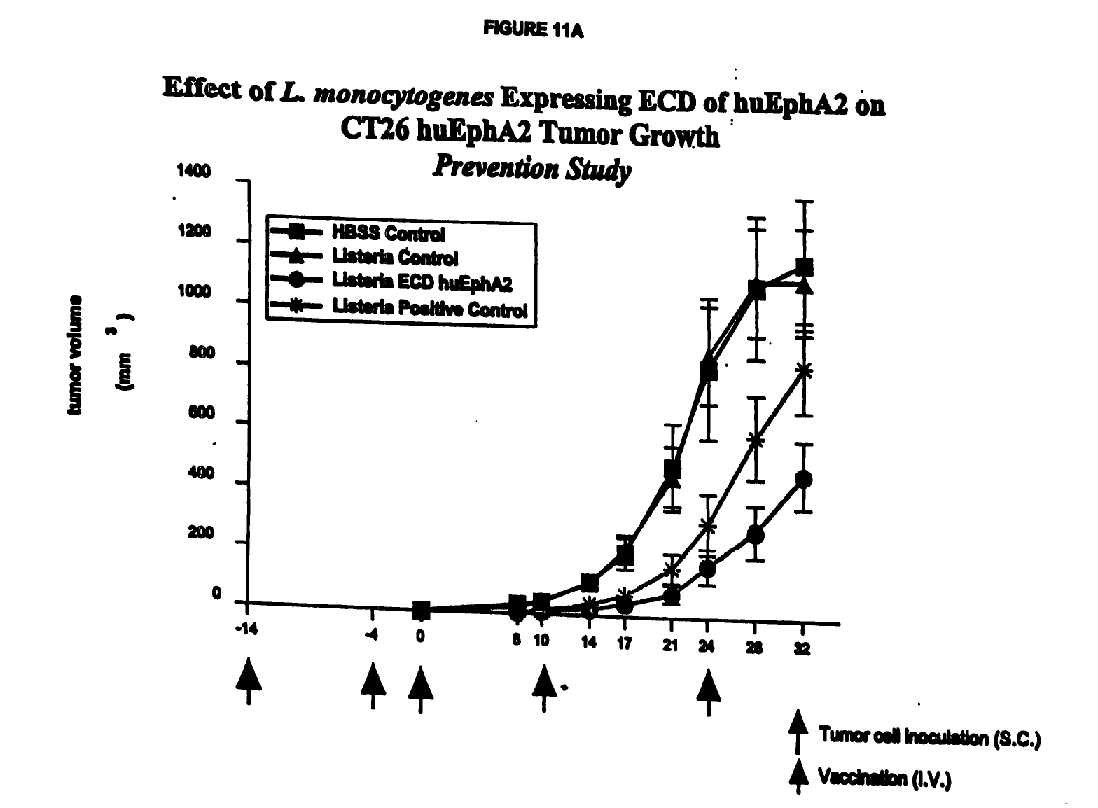

FIG. 10A demonstrates the anti-tumor efficacy of Listeria expressing the ECD of hEphA2 against s.c. inoculations of huEphA2-expressing CT26 cells as compared to the negative controls (*p=0.0012). The data are summarized in Table 8 below:

TABLE 8

| | Tumor Volume | | P vs. |
|---|---|---|---|
| | (mm³ ± s.e.m.) | P vs. | Listeria |
| Vaccination Group | (Day 42) | HBSS | Control |
| HBSS | 1202.9 (±321) | — | 0.5528 |
| Listeria Control | 945.5 (±338) | 0.5528 | — |
| Listeria-AH1/A5 | 392.5 (±225) | 0.0471 | 0.1895 |
| Listeria-hEphA2-ECD | 0.0 (±0.0) | 0.0012 | 0.0118 |

FIG. 10B demonstrates the anti-tumor efficacy of Listeria expressing the ECD of hEphA2 against i.v. inoculations of huEphA2-expressing CT26 cells as compared to the negative controls *p=0.0017). The data are summarized in Table 9 below:

TABLE 9

| | Median | | |
|---|---|---|---|
| | Survival | | # Survivors |
| Vaccination Group | (Days) | P vs. HBSS | (Day 65) |
| HBSS | 18 | — | 0 |
| Listeria Control | 18 | 0.754 | 0 |
| Listeria-AH1/A5 | >65 | 0.0017 | 5 |
| Listeria-hEphA2-ECD | >65 | 0.0017 | 3 |

Preventive studies were performed according to the schedule described below. These studies utilized a pool of CT26 cells expressing huEphA2 generated by the single cell FACS assays described above.

Groups: Eight groups of ten mice per group. Groups 1-4 were inoculated s.c. and groups 5-8 were inoculated i.v. with CT26 colon carcinoma cells transfected with human EphA2, as shown in Table 10 below:

TABLE 10

| | Number of Mice per |
|---|---|
| Treatment Group | Groups |
| 1. Control - HBSS | 10 |
| 2. L4029 - control Listeria monocytogenes | 10 |
| 3. L4029-EphA2 exFlag - Listeria monocytogenes | 10 |
| expressing extracellular domain of human EphA2 | |
| 4. L4029 - AH1 Listeria monocytogenes | 10 |
| 5. Control - HBSS | 10 |
| 6. L4029 - control Listeria monocytogenes | 10 |
| 7. L4029-EphA2 exFlag - Listeria monocytogenes | 10 |
| expressing extracellular domain of human EphA2 | |
| 8. L4029 - AH1 Listeria monocytogenes | 10 |

Schedule: Animals received i.v. administrations of the agents listed above in 200 μl bolus on Day 0 and Day 10. On Day 14, animals were inoculated with CT26 colon carcinoma cells transfected with human EphA2 (L4029EphA2-exFlag), Listeria control (L4029), or Listeria positive control containing the AH1 protein (L4029-AH1) (5×10⁵ cells in 100 μl volume) either subcutaneously or intravenously (experimental lung metastases model). Tumor volume was measured bi-weekly (s.c inoculation only) and animal weights assessed on a weekly basis. Any animals possessing tumors greater than 2000 mm³ or demonstrating signs of morbidity (hunched posture, impaired breathing, decreases mobility, greater than 20% weight loss, etc.) were humanely euthanized. The experimental schedule is summarized in Table 11 below:

TABLE 11

| | Cell Inoculation | | |
|---|---|---|---|
| | Route | Primary | Boost |
| | (5 × 10⁵ cell in 100 μl) | Vaccination | Vaccination |
| Group | (Day 14) | (Day 0) | (Day 10) |
| 1. Control | s.c. | HBSS | HBSS |
| 2. L4029 | s.c. | 2 × 10⁷ CFU | 2 × 10⁷ CFU |
| 3. L4029 EphA2- | s.c. | 2 × 10⁷ CFU | 2 × 10⁷ CFU |
| exFlag | | | |
| 4. L4029 - AH1 | s.c. | 2 × 10⁷ CFU | 2 × 10⁷ CFU |
| 5. Control | i.v. | HBSS | HBSS |
| 6. L4029 | i.v. | 2 × 10⁷ CFU | 2 × 10⁷ CFU |
| 7. L4029 EphA2- | i.v. | 2 × 10⁷ CFU | 2 × 10⁷ CFU |
| exFlag | | | |
| 8. L4029 - AH1 | i.v. | 2 × 10⁷ CFU | 2 × 10⁷ CFU |

In this study, vaccination with Listeria-huEphA2 exFlag demonstrated a significant anti-tumor effect in both the s.c. and experimental lung metastases models (i.v.).

In the s.c. model, a significant reduction in tumor growth was achieved with 3 mice remaining tumor-free. This response was also specific compared to the control Listeria and vehicle treated animals. In the experimental lung metastases model, vaccination with Listeria huEphA2-exFlag also demonstrated efficacy compared to the vehicle treated group.

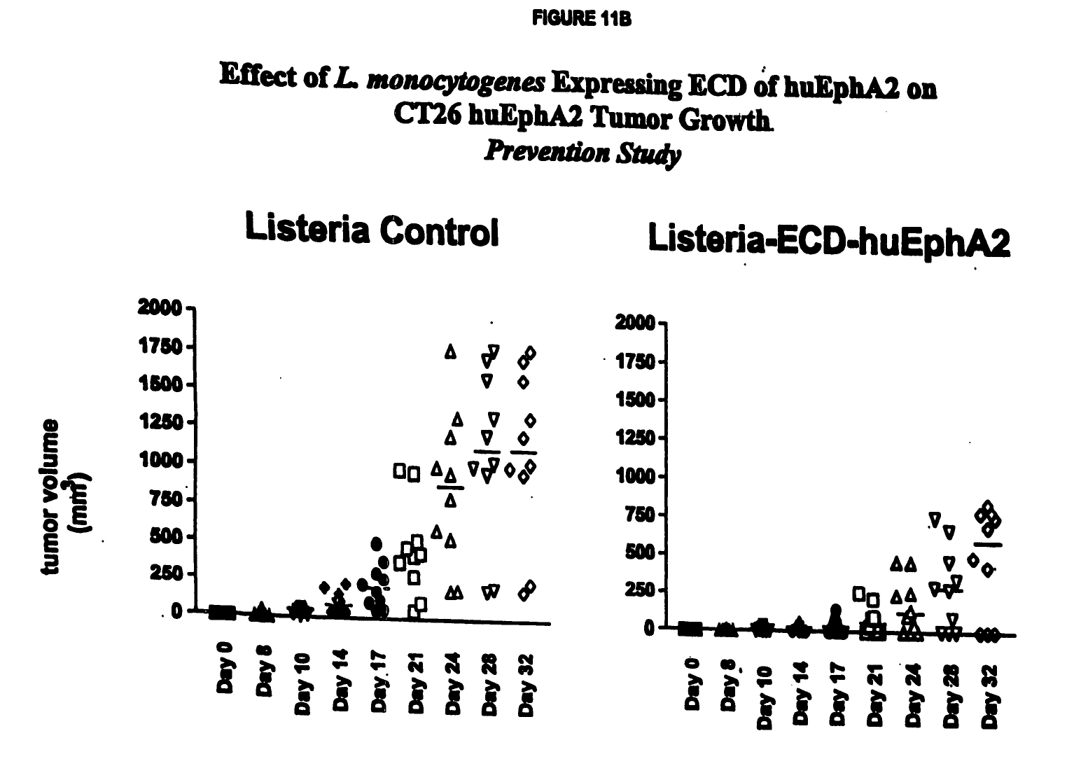

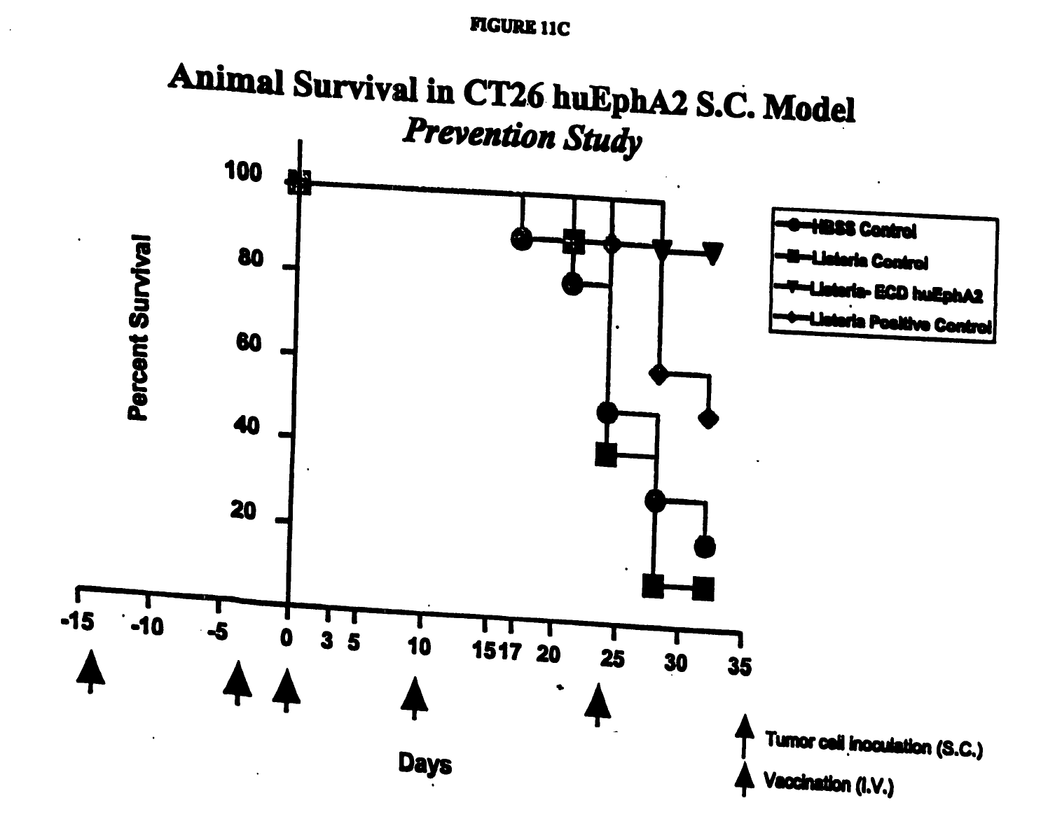

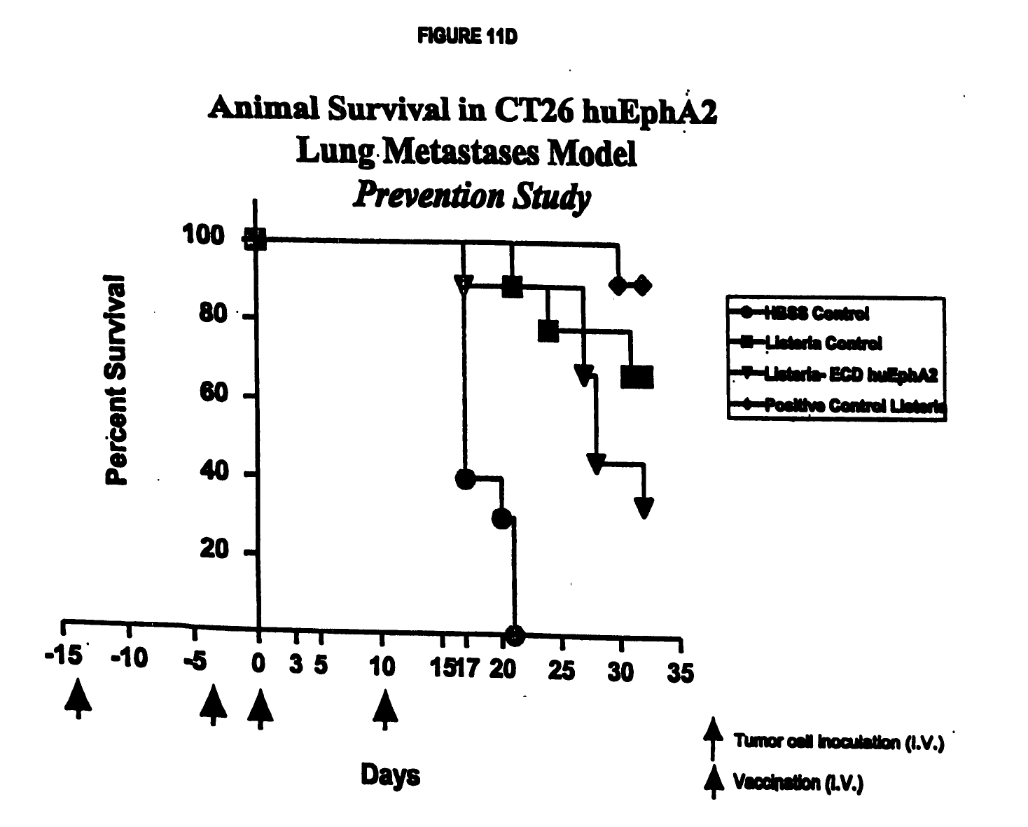

FIGS. 11A-11D illustrate results of the preventive experiments. FIG. 11A shows that the tumor volume of mice inoculated with CT26 cells expressing the ECD of huEphA2 was significantly reduced when compared to vehicle (HBSS), Listeria (L4029) and Listeria positive (L4029-AH1) controls starting at day 21 and continued until day 32 post inoculation. FIG. 11B also depicts results of the preventive experiments, showing again that the tumor volume of mice inoculated with CT26 cells expressing the ECD of huEphA2 (L4029-EphA2 exFlag) was significantly reduced when compared to the Listeria (L4029) control starting at day 21 and continued until day 32 post inoculation. FIG. 11C illustrates the results of the prevention study in the s.c. model, measuring percent survival of the mice post CT26 tumor cell inoculation. Compared to all control groups, the L4029-EphA2 exFlag group had the most significant survival rate (indicated by triangles). FIG. 11D illustrates the results of the prevention study in the lung metastases model, measuring the percent survival of the mice post tumor cell inoculation. Compared to all control groups, the L4029-EphA2 exFlag group had the most significant survival rate.

The foregoing data demonstrate that preventative immunization with Listeria expressing the ECD of hEphA2 suppresses CT26-hEphA2 tumor growth and increases survival.

6.5.3.2. Effect of Immunization with Listeria Expressing ICD of huEphA2 on the Survival of Mice Inoculated with RenCa-hEphA2

Preventive studies were performed utilizing a pool of RenCa cells (American Type Culture Collection, Manassas, Va.) expressing huEphA2 generated and screened by the methods described above. Groups of ten Balb/c mice per group were inoculated subcutaneously with RenCa renal cell carcinoma cells expressing human EphA2 (“RenCa-hEphA2 cells”). The mice were immunized with 0.1 LD50 Listeria control or Listeria expressing the ICD of hEphA2 in a 200 ml bolus. The immunizations were performed 18 and 4 days prior to RenCa-hEphA2 cell tumor challenge. Tumor volume measurements were obtained twice weekly for the course of the study to determine an anti-tumor effect of the vaccinations.

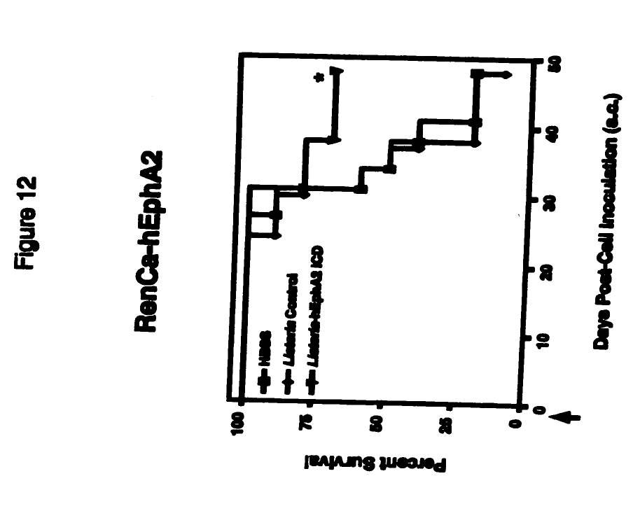

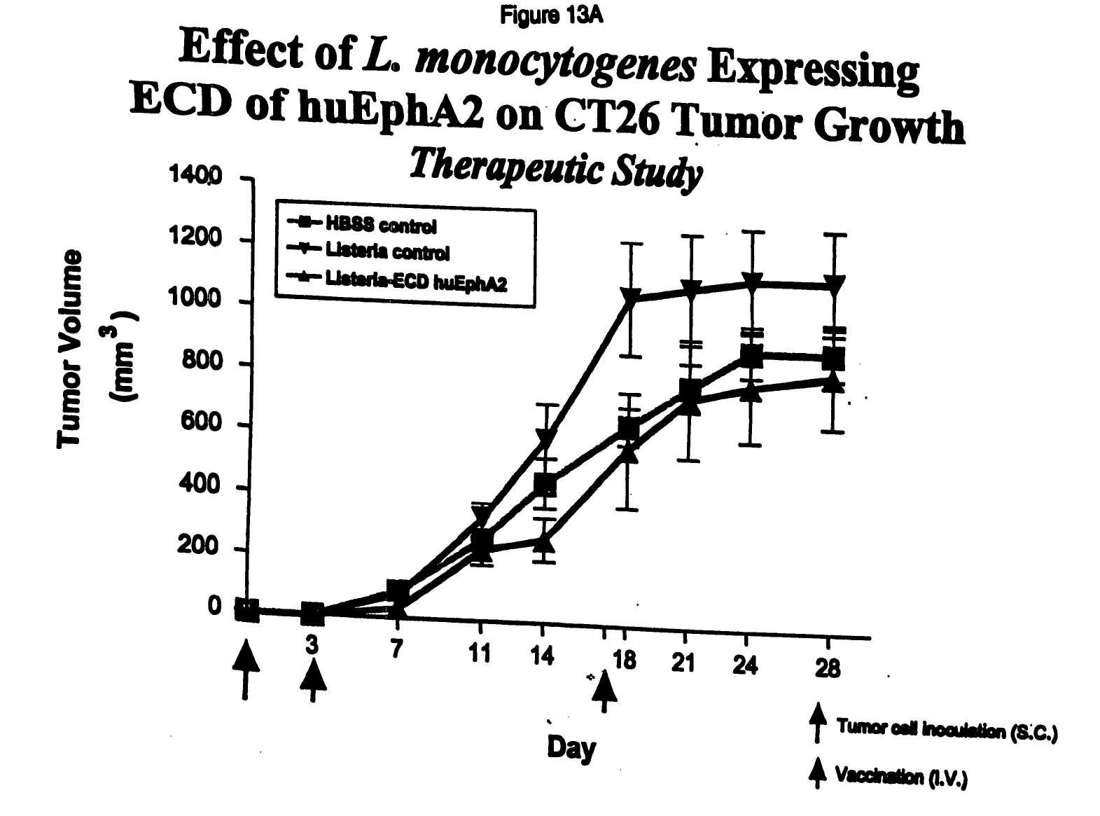

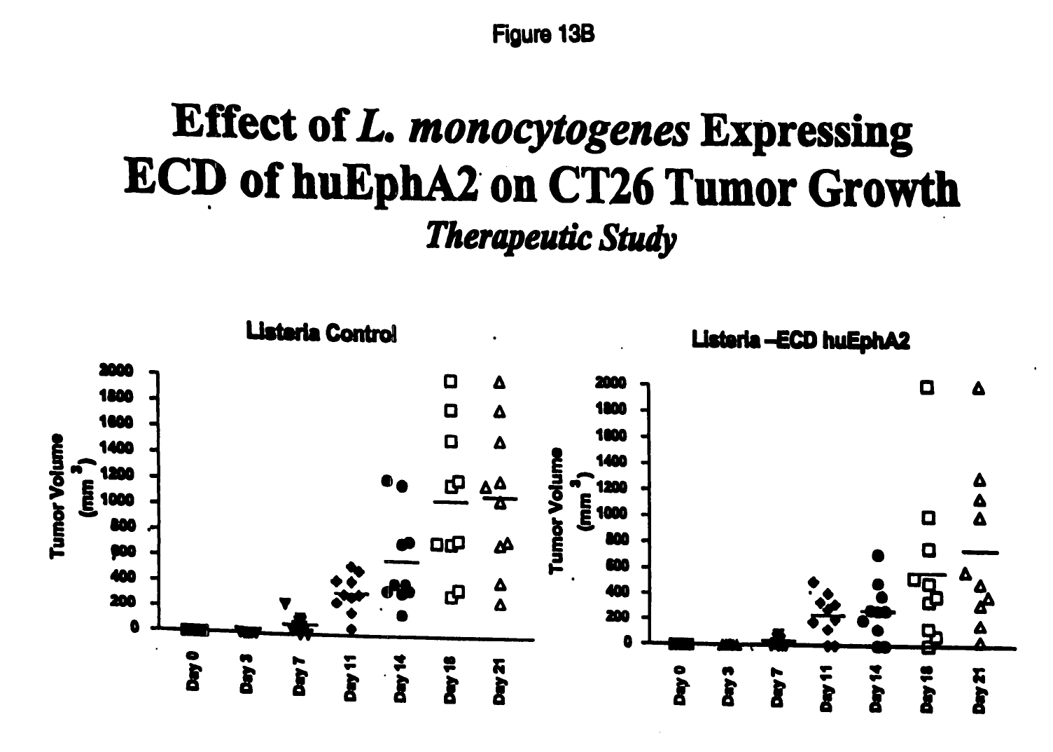

FIG. 12 demonstrates the anti-tumor efficacy of Listeria expressing the ICD of hEphA2 against s.c. inoculations of huEphA2-expressing RenCA cells as compared to the negative controls. A significant anti-tumor response, as assessed by increased survival via Kaplan-Meier analysis, was observed in animals vaccinated with Listeria expressing the ICD of hEphA2 as compared to animals vaccinated with Listeria alone (*p=0.0079).

6.5.4. Therapeutic EphA2 Vaccinations

Therapeutic studies were performed utilizing a pool of CT26 cells expressing huEphA2 generated by the single cell FACS assays described above.

A representative therapeutic study was performed as follows:

Groups: Six groups of ten mice per group. Groups 1-3 were inoculated s.c. and groups 4-6 were inoculated i.v. with CT26 murine colon carcinoma cells, as shown in Table 12 below:

TABLE 12

| | Number of Mice per |
|---|---|
| Treatment Group | Groups |
| 1. Control - HBSS | 10 |
| 2. L4029 - control Listeria monocytogenes | 10 |
| 3. L4029-EphA2 exFlag - Listeria monocytogenes | 10 |
| expressing extracellular domain of human EphA2 | |
| 4. Control - HBSS | 10 |
| 5. L4029 - control Listeria monocytogenes | 10 |
| 6. L4029-EphA2 exFlag - Listeria monocytogenes | 10 |
| expressing extracellular domain of human EphA2 | |

Schedule: Animals were inoculated with CT26 colon carcinoma cells transfected with human EphA2 (L4029-EphA2 exFlag), Listeria control (L4029-control) or vehicle (HBSS) (5×10⁵ cells in 100 ml volume) either subcutaneously or intravenously (experimental lung metastases model). Three days after cell inoculation, animals received i.v. administrations of the agents listed above in 200 ml bolus. Two weeks following the first administration, the animals received a booster vaccination. Tumor volume was measured bi-weekly (s.c inoculation only) and animal weights assessed on a weekly basis. Any animals possessing tumors greater than 2000 mm³ or demonstrating signs of morbidity (hunched posture, impaired breathing, decreases mobility, greater than 20% weight loss, etc.) were humanely euthanized. The schedule is summarized in Table 13 below.

TABLE 13

| | Cell | | |
|---|---|---|---|
| | Inoculation | | |
| | Route | | Boost |
| | (5 × 10⁵ cell | Primary Vaccination | Vaccination |
| Group | in 100 μl) | (Day 3) | (Day 17) |
| 1. Control | s.c. | HBSS | HBSS |
| 2. L4029 | s.c. | 6 × 10⁶ to 2 × 10⁷ | 6 × 10⁶ to 2 × 10⁷ |
| | | CFU | CFU |
| 3. L4029 EphA2- | s.c. | 6 × 10⁶ to 2 × 10⁷ | 6 × 10⁶ to 2 × 10⁷ |
| exFlag | | CFU | CFU |
| 4. Control | i.v. | HBSS | HBSS |
| 5. L4029 | i.v. | 6 × 10⁶ to 2 × 10⁷ | 6 × 10⁶ to 2 × 10⁷ |
| | | CFU | CFU |
| 6. L4029 EphA2- | i.v. | 6 × 10⁶ to 2 × 10⁷ | 6 × 10⁶ to 2 × 10⁷ |
| exFlag | | CFU | CFU |

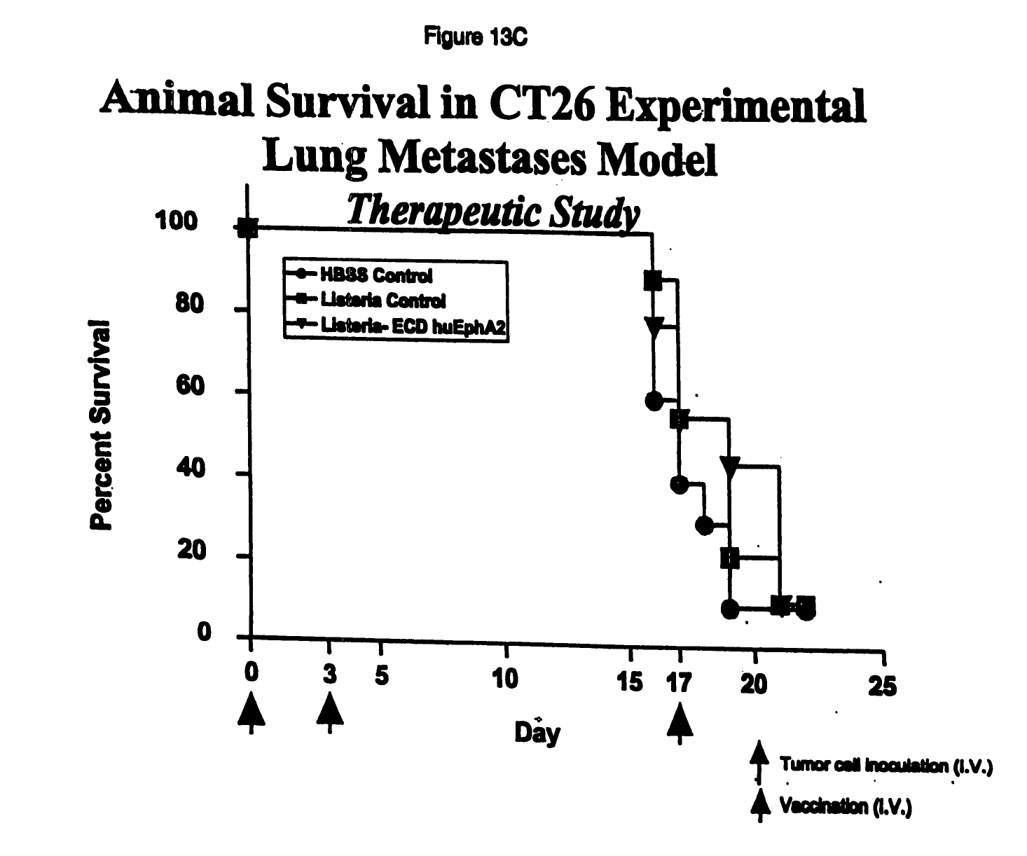

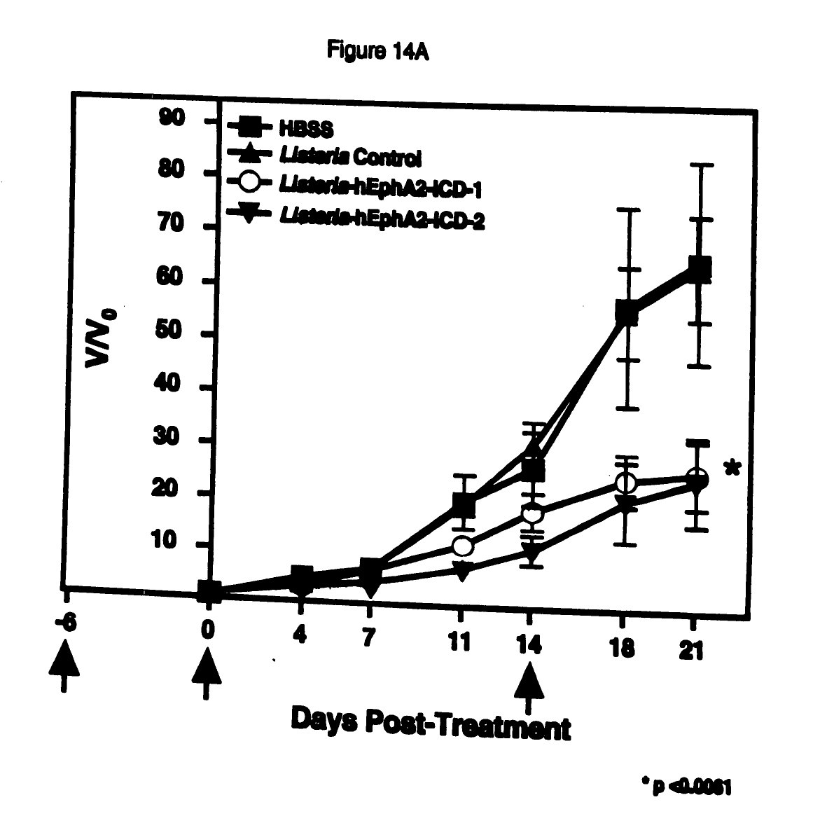

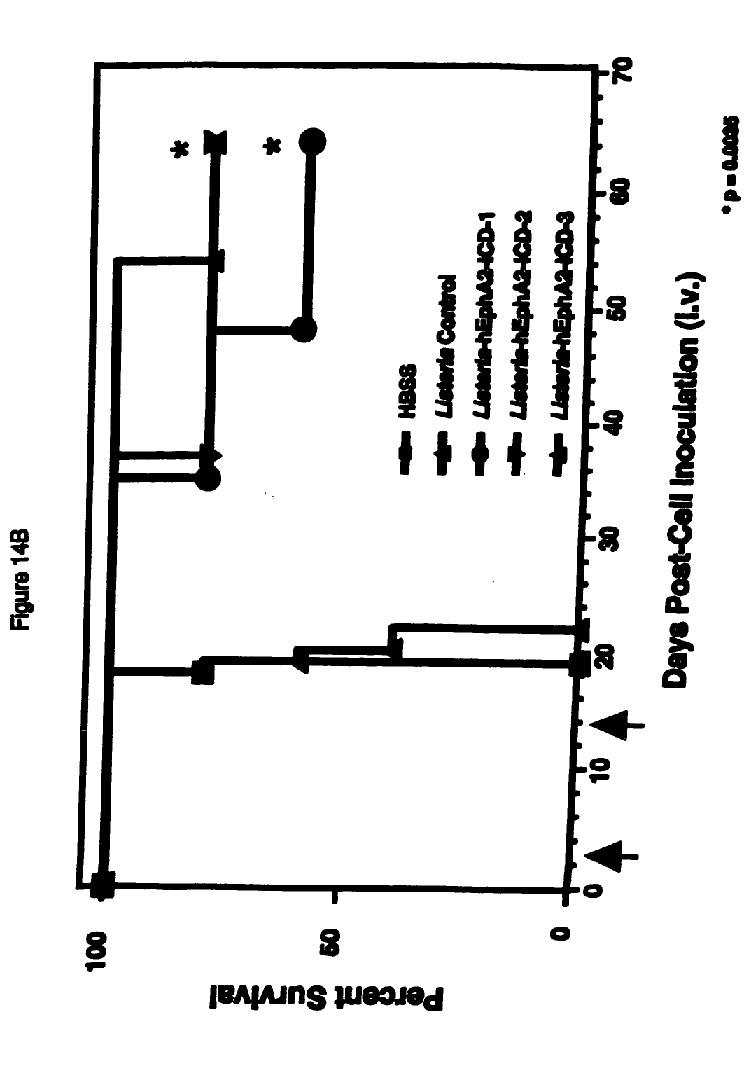

FIGS. 13A-13C illustrate the results of a typical therapeutic study. In FIG. 13A, tumor volume was measured at several intervals post inoculation. Compared to the HBSS and Listeria controls, the mice inoculated with CT26 cells expressing the ECD of huEphA2 had a significantly lower tumor volume after day 14 and continued onto day 28. FIG. 13B depicts the mean tumor volume of mice inoculated with CT26 cells containing either Listeria control or huEphA2. Compared to control, the mice inoculated with CT26 cells expressing huEphA2 had a reduced mean tumor volume. FIG. 13C represents the results of the therapeutic study using the lung metastases model, measuring percent survival of the mice post inoculation with CT26 cells with either HBSS or Listeria control, or Listeria expressing the ECD of huEphA2. Animals inoculated with CT26 cells expressing the ECD of huEphA2 (depicted by triangles) showed a higher percent survival rate compared to controls.

In another study, groups of ten Balb/c mice per group were inoculated s.c. or i.v. with CT26 colon carcinoma cells transfected with human EphA2 (“CT26-hEphA2”).

The mice were immunized with 0.1 LD₅₀ actA Listeria control or Listeria expressing the ICD of hEphA2 in a 200 μl bolus. In one regimen, the immunizations were performed 6 and 14 days post s.c. CT26-hEphA2 tumor inoculation. In another regimen, the immunizations were performed 3 and 14 days post i.v. CT26-hEphA2 tumor inoculation. Anti-tumor efficacy was determined from twice weekly tumor measurements and survival.

Significant anti-tumor efficacy was observed in the Listeria-hEphA2 vaccinated animals (p=0.0035).

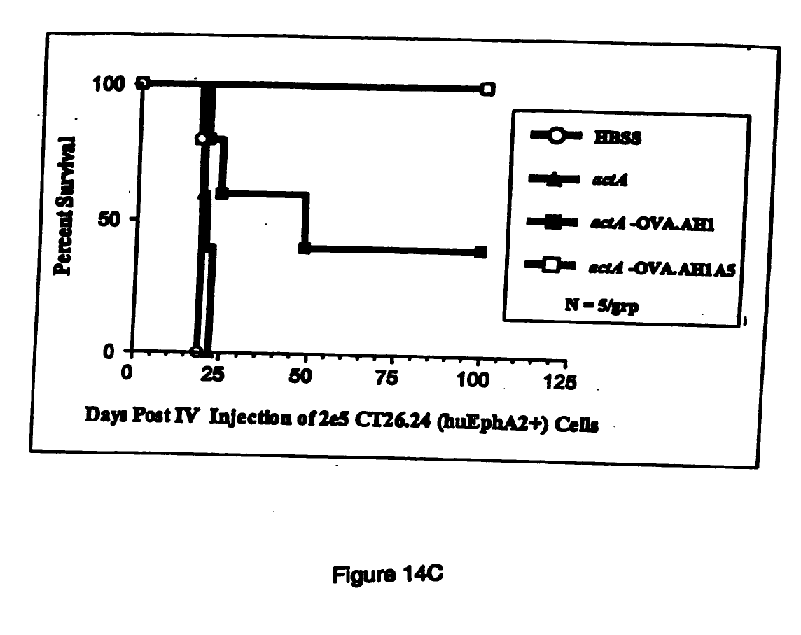

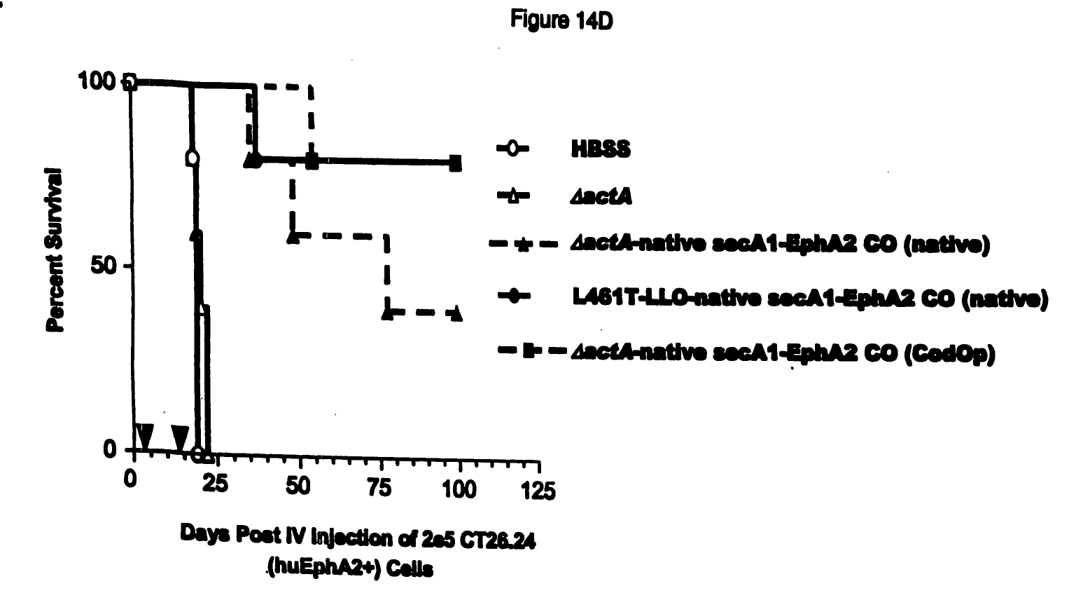

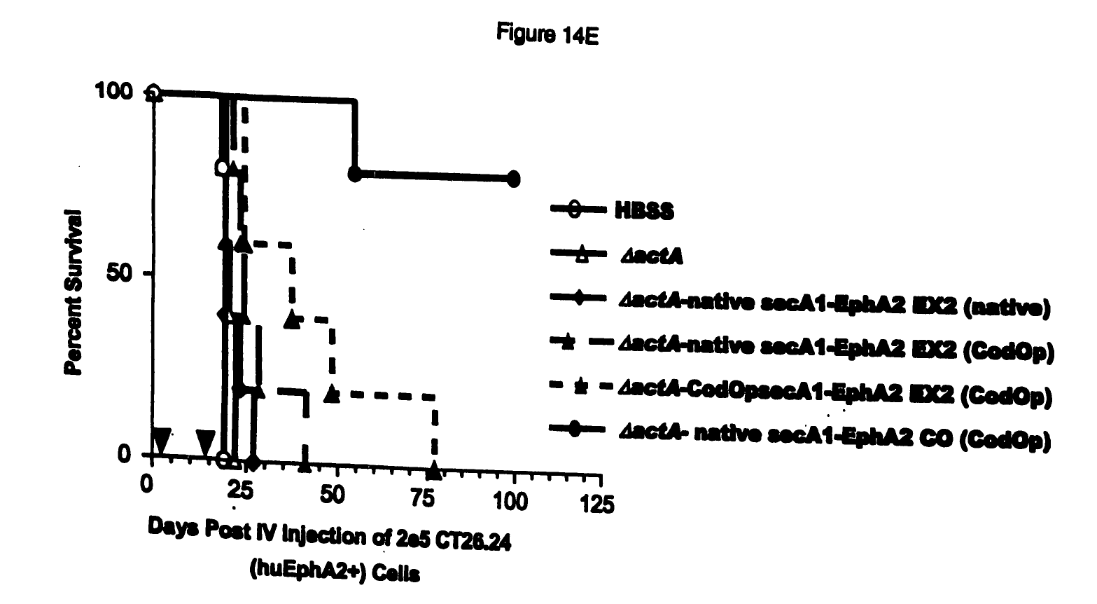

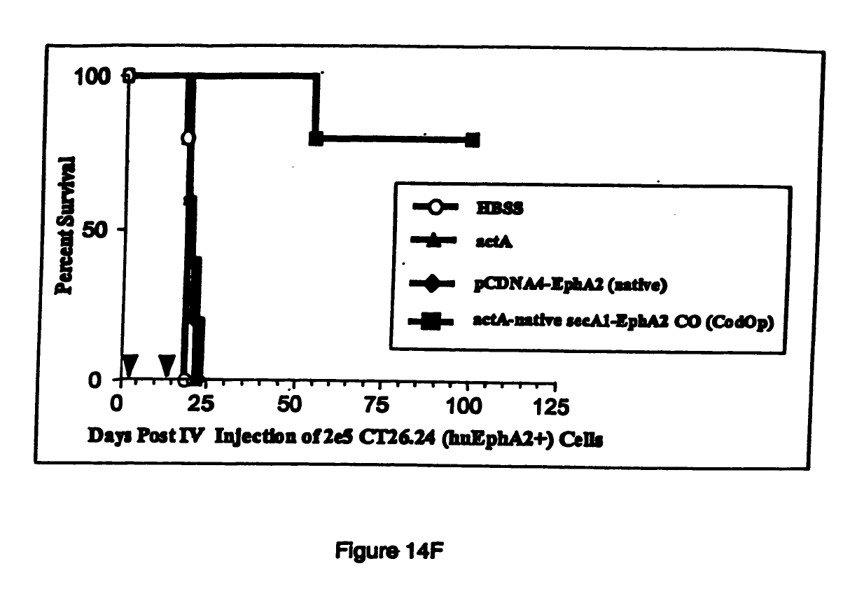

FIG. 14A demonstrates the tumor measurements of immunized animals. This data is summarized in Table 14 below:

TABLE 14

| | Tumor Volume | | P vs. |
|---|---|---|---|
| | (mm³ ± s.e.m.) | P vs. | Listeria |
| Vaccination Group | (Day 21) | HBSS | Control |
| HBSS | 1827 (±518) | — | 0.961 |
| Listeria Control | 1799 (±267) | 0.961 | — |
| Listeria-AH1/A5 | 0 | 0.0005 | 0.000003 |
| Listeria-hEphA2-ICD-1 | 694 (±232) | 0.0054 | 0.006 |
| Listeria-hEphA2-ICD-2 | 731 (±176) | 0.052 | 0.004 |

FIG. 14B demonstrates the survival time of immunized animals. This data is summarized in Table 15 below:

TABLE 15

| | Median | | |
|---|---|---|---|
| | Survival | | # Survivors |
| Vaccination Group | (Days) | P vs. HBSS | (Day 65) |
| HBSS | 19 | — | 0 |
| Listeria Control | 20 | Ns | 0 |
| Listeria-hEphA2-ICD-1 | >65 | 0.0035 | 3 |
| Listeria-hEphA2-ICD-2 | >65 | 0.0035 | 4 |
| Listeria-hEphA2-ICD-3 | >65 | 0.0035 | 4 |

Immunization of Balb/C mice bearing CT26.24 (huEphA2+) lung tumors with recombinant Listeria encoding OVA.AH1 (MMTV gp70 immunodominant epitope) or OVA.AH1-A5 (MMTV gp70 immunodominant epitope, with heteroclitic change for enhanced T-cell receptor binding) confers long-term survival (FIG. 14C).

The EphA2 CO domain is strongly immunogenic, and a significant long term increase in survival of Balb/C mice bearing CT26.24 (huEphA2+) lung tumors was observed when immunized with recombinant Listeria encoding codon-optimized or native EphA2 CO domain sequence (FIG. 14D).

The EphA2 EX2 domain is poorly immunogenic, and increased survival of Balb/C mice bearing CT26.24 (huEphA2+) lung tumors was observed only when immunized with recombinant Listeria encoding codon-optimized secA1 signal peptide fused with the codon-optimized EphA2 EX2 domain sequence. Therapeutic efficacy was not observed in mice when immunized with recombinant Listeria encoding native secA1 signal peptide fused with the codon-optimized EphA2 EX2 domain sequence (FIG. 14E). The desirability of using both codon-optimized secA1 signal peptide and EphA2 EX2 domain sequences was supported by statistically significant therapeutic anti-tumor efficacy, as shown in the table below:

A comparison by log-rank test of survival curves shown in FIG. 14E and summarized in Table 16 below:

TABLE 16

| | | | Significance versus |
|---|---|---|---|
| | | Significance | actA-native |
| | Median | versus | secA1/EphA2 EX2 |
| | Survival | HBSS cohort | cohort |
| Experimental Group | (Days) | (p value) | (p value) |
| HBSS | 19 | — | — |
| ActA | 20 | NS | NS |
| actA-native secA1- | 19 | NS | — |
| EphA2 EX2 (native) | | | |
| actA-native secA1- | 24 | 0.0035 | NS |
| EphA2 EX2 (CodOp) | | | |
| actA-CodOp secA1- | 37 | 0.0035 | 0.0162 |
| EphA2 EX2 (CodOp) | | | |
| actA-native secA1- | >99 | 0.0035 | 0.0015 |
| EphA2 CO (CodOp) | | | |

Significantly, even though pCDNA4-EphA2 plasmid transfected 293 cells yielded very high levels of protein expression, immunization of Balb/C mice bearing CT26.24 (huEphA2+) lung tumors with the pCDNA4-EphA2 plasmid did not result in any observance of therapeutic anti-tumor efficacy (FIG. 14F).

For therapeutic in vivo tumor studies, female Balb/C mice were implanted IV with 5×10⁵ CT26 cells stably expressing EphA2. Three days later, mice were randomized and vaccinated IV with various recombinant Listeria strains encoding EphA2. In some cases (noted in figures) mice were vaccinated with 100 μg of pCDNA4 plasmid or pCDNA4-EphA2 plasmid in the tibialis anterior muscle. As a positive control, mice were vaccinated IV with recombinant Listeria strains encoding OVA.AHI or OVA.AH1-A5 protein chimeras. Mice were vaccinated on days 3 and 14 following tumor cell implantation. Mice injected with Hanks Balanced Salt Solution (HBSS) buffer or unmodified Listeria served as negative controls. All experimental cohorts contained 5 mice. For survival studies mice were sacrificed when they started to show any signs of stress or labored breathing.

The foregoing data demonstrate that therapeutic immunization with Listeria expressing the hEphA2 suppresses established CT26-hEphA2 tumor growth and increases survival.

### 6.6. Example 6

### Long-Term Suppression of CT26-hEphA2 Tumor Growth Upon Rechallenge

Balb/c mice failing to form tumors after preventative immunization with Listeria expressing either the ICD or ECD of hEphA2 against CT26-hEphA2 tumor challenged, were re-challenged (s.c.) with both CT26 parental cell line and CT26-hEphA2 cells on opposite flanks 56 days after initial tumor challenge and 60 days after the last immunization. Age-matched mice were used as a control in this experiment.

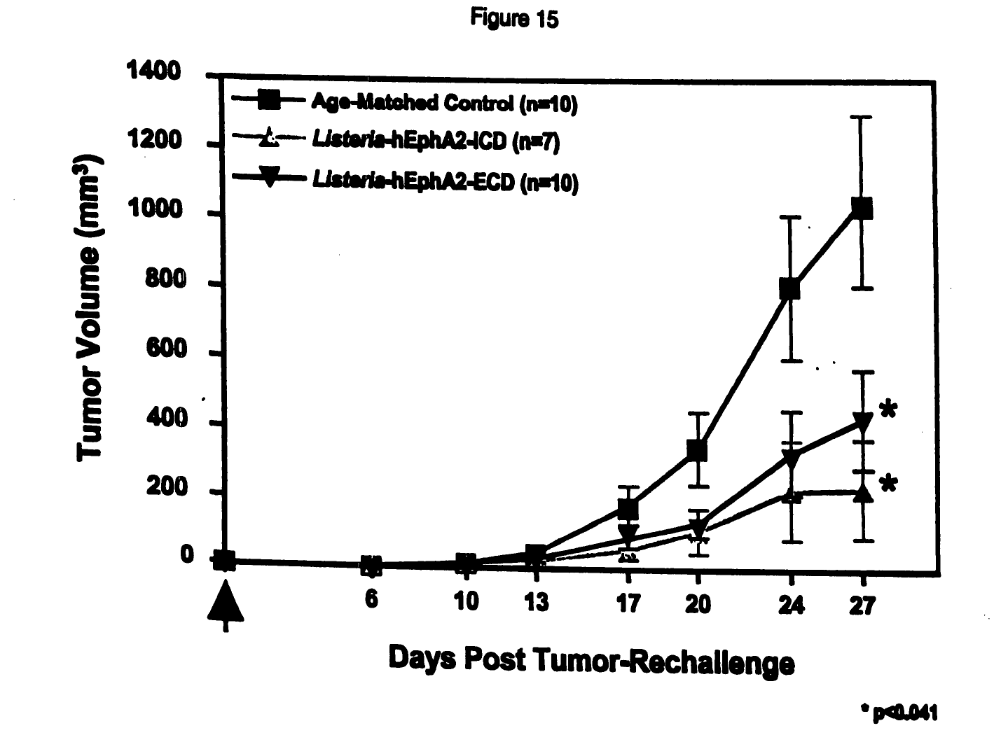

Re-challenge with parental CT26 cells showed no statistically significant differences in tumor growth between groups (data not shown). However, as shown in FIG. 15, both groups vaccinated with Listeria expressing either the ICD or ECD of hEphA2 demonstrated a significant suppression of tumor growth upon re-challenge (*p<0.041).

### 6.7. Example 7

### Immunization with Listeria Expressing hEphA2 Elicits an EphA2-Specific CD8+ T Cell Response

Balb/c mice (n=3) were immunized with Listeria L461T expressing the intracellular domain of hEphA2 (hEphA2-ICD) or ΔactA expressing codon optimized extracellular domain of hEphA2 (hEphA2-ECD) two weeks apart. Mice were euthanized, and spleens harvested and pooled 6 days after the last immunization. For the ELISPOT assay, the cells were re-stimulated in vitro with P815 cells expressing full-length hEphA2 or cell lysates prepared from these cells. The parental P815 cells or cell lysates served as a negative control. Cells were also stimulated with recombinant hEphA2 Fc fusion protein. IFN-gamma positive spot forming colonies (SFCs) were measured using a 96 well spot reader.

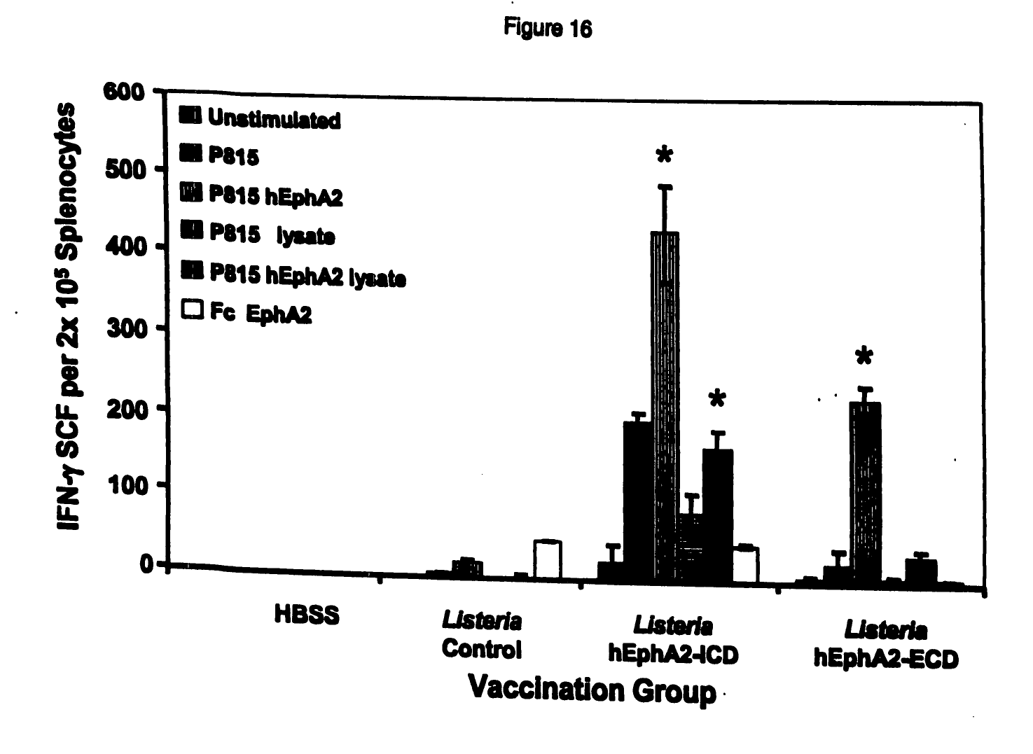

As shown in FIG. 16, increased IFN-gamma SFCs were observed with spleen cells derived from mice vaccinated with Listeria-hEphA2. Both hEphA2 expressing cells or cell lysates stimulation resulted in an increase in IFN-gamma SFC which suggests an EphA2-specific CD8+ as well as CD4+ T cell response. Spleen cells from mice vaccinated with the parental Listeria control did not demonstrate an increase in IFN-gamma SFC.

### 6.8. Example 8

### Both CD4+ and CD8+ T Cell Responses are Required for Maximal hEphA2-Directed Anti-Tumor Efficacy

Balb/c mice (n=10) were inoculated i.v. with 2×10⁵ CT26-hEphA2 on day 0. CD4+ cells and CD8+ T-cells were depleted by injecting 200 μg anti-CD4 (ATCC hybridoma GK1.5) or anti-CD8 (ATCC hybridoma 2.4-3) on Days 1 and 3, which was confirmed by FACS analysis (data not shown). Mice were then immunized i.v. with 0.1 LD₅₀ Listeria L461 T expressing hEphA2 ICD on Day 4 and monitored for survival.

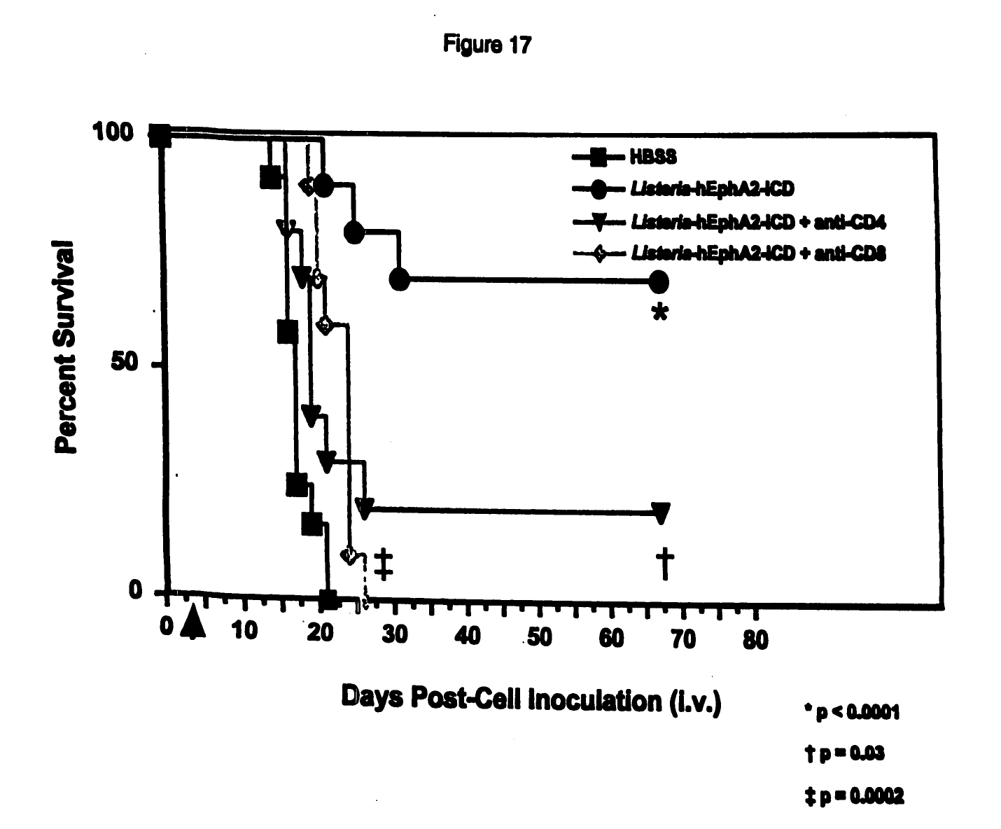

As shown in FIG. 17, both CD4+ and CD8+ depleted groups failed to demonstrate the degree of anti-tumor response seen in the non-T cell depleted animals. The data are summarized in Table 17 below:

TABLE 17

| | Median | | |
|---|---|---|---|
| | Survival | | # Survivors |
| Vaccination Group | (Days) | P vs. HBSS | (Day 67) |
| HBSS | 17 | — | 0 |
| Listeria-hEphA2-ICD | >67 | <0.0001 | 7 |
| Listeria-hEphA2-ICD + anti- | 19 | 0.03 | 2 |
| CD4 | | | |
| Listeria-hEphA2-ICD + anti- | 24 | 0.0002 | 0 |
| CD8 | | | |

The foregoing data indicate a requirement for both CD4+ and CD8+ T cells in optimal suppression of tumor growth.

### 6.9. Example 9

### Therapeutic Vaccination with Listeria Expressing Human EphA2 ICD Enhances CD45+ Tumor Infiltrate

Balb/c mice (n=3) were immunized with 0.1 LD50 actA-Listeria control or Listeria expressing either the ECD or ICD of hEphA2, 6 days post s.c. CT26-hEphA2 tumor inoculation. 9 days post-vaccination, tumors were harvested, fixed in 10% neutral buffered formalin, embedded in paraffin and sectioned at 4 μm. Microscope slides were prepared from the tumor sections. The tissues on the slides were deparaffinized and rehydrated as follows: 4 changes with xylene, 5 minutes each; 2 changes with absolute alcohol, 5 minutes each; 1 change with 95% alcohol for 5 minutes; 1 change with 70% for 5 minutes; and two changes with distilled water.

Steam antigen retrieval was performed in a Black and Decker Rice steamer using target antigen retrieval (TAR) solution (DakoCytomation, Carpinteria, Calif.) using a modification of the manufacture's protocol. The slides were placed into TAR solution preheated to just below boiling temperature and incubated for 20 minutes. The slides were then removed from the TAR solution and allowed to cool at room temperature for 20 minutes, and rinsed twice in TBS assay buffer.

Staining of the slides with biotinylated antibody was performed as follows:

Endogenous peroxidase was blocked by immersing the slides in solution of 3% hydrogen peroxide in methanol, for 10 minutes, followed with 2 changes of distilled water, 5 minutes each. Protein was blocked by immersing the slides in a solution of 5% Bovine Serum Albumin (BSA) in 1× Tris buffered saline with 0.01% Tween 20 (TBST) for at least 30 minutes.

After wiping excess BSA solution from the slide, creating a “pool”, centered around tissue, the slide was laid flat in humid chamber and biotinylated rat anti-mouse CD45/B220 (Pharmingen) at 1:100 dilution in a solution of 1% BSA/TBST was applied. The slide was incubated in a humid chamber overnight at room temperature with care taken to prevent drying of the tissue sections.

The next morning, the slides are washed with 2 changes of TBST, the second one lasting 10 minutes. Streptavidin conjugated with either HRP or AP is applied, incubating for 30 minutes at room temp. The slides are washed with two changes of TBST, visualized with an appropriate substrate chromagen (for Strep-HRP, DAB is used). After a wash in distilled water, the slides are counterstained with Mayers Hematoxylin by immersing the slides in dye for 2 minutes. The slides are then washed in running tap water until water runs clear, immersed in bluing agent (Scotts substitute tap water) for 30 seconds, and washed again in tap water. The slides are dehydrated and cleared in graded alcohols through xylene (or xylene substitute) by the following washes: 95% alcohol for 1 minute, 3 changes absolute alcohol for 1 minute each, and 4 changes xylenes for 1 minute each.

Mounting media is applied to the cover slips (for xylene, DPX mountant is used) and the slides are allowed to dry over night prior to visualization.

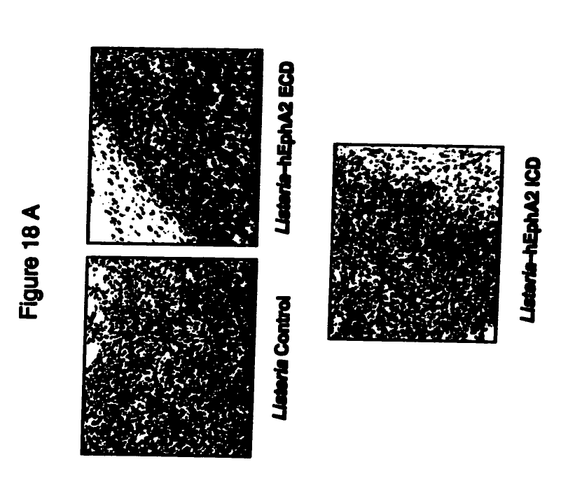

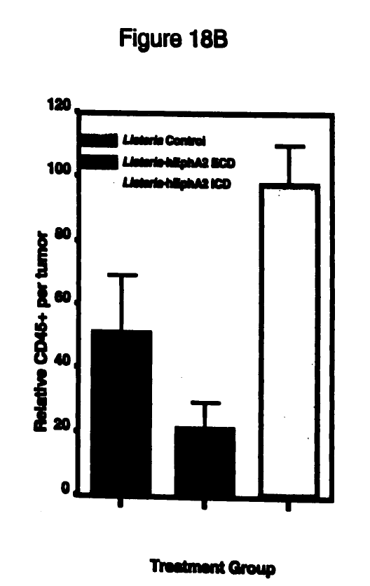

The sections were visualized on a Nikon Eclipse E400 and images captured with a Nikon DXM1200 digital camera (FIG. 18A). Data was further normalized to tumor volume (FIG. 18B).

The results demonstrate that tumor associated infiltrating lymphocytes are increased following therapeutic vaccination.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method of eliciting an immune response against an EphA2-expressing cell in a subject, said method comprising administering to a subject a composition comprising a Listeria bacterium that expresses an EphA2 antigenic peptide in an amount effective to elicit an immune response against an EphA2-expressing cell.

2. The method of claim 1, wherein the Listeria is Listeria monocytogenes.

3. The method of claim 2, wherein the Listeria is attenuated.

4. The method of claim 1, wherein the nucleic acid encoding the EphA2 antigenic peptide comprises a nucleotide sequence encoding a secretory signal operatively linked to the sequence encoding the EphA2 antigenic peptide.

5. A method of claim 1, wherein the subject has cancer.

6. The method of claim 5, wherein said cancer is of an epithelial cell origin.

7. The method of claim 5, wherein said cancer is of a T cell origin.

8. The method of claim 6, wherein said cancer is cancer of the skin, lung, colon, breast, prostate, bladder or pancreas or is a renal cell carcinoma or melanoma.

9. The method of claim 7, wherein said cancer is a leukemia or a lymphoma.

10. The method of claim 1, wherein the subject has a non-neoplastic hyperproliferative disorder.

11. The method of claim 10, wherein the hyperproliferative disorder is an epithelial cell disorder.

12. The method of claim 11, wherein the hyperproliferative disorder is asthma, chronic pulmonary obstructive disease, lung fibrosis, bronchial hyper responsiveness, psoriasis, and seborrheic dermatitis.

13. A method of treating a human subject having a hyperproliferative disorder of EphA2-expressing cells, said method comprising administering to the subject a composition comprising an EphA2 antigenic peptide-expressing Listeria bacterium in an amount effective to treat a hyperproliferative disorder of EphA2-expressing cells.

14. The method of claim 13, wherein the Listeria is Listeria monocytogenes.

15. The method of claim 13, wherein the subject has cancer.

16. The method of claim 15, wherein the cancer is of an epithelial cell origin.

17. The method of claim 15, wherein the cancer is of an endothelial cell origin.

18. The method of claim 15, wherein the cancer is of a T cell origin.

19. The method of claim 15, wherein said cancer comprises cells that overexpress EphA2 relative to non-cancer cells having the tissue type of said cancer cells.

20. The method of claim 16, wherein said cancer is cancer of the skin, lung, colon, breast, prostate, bladder or pancreas or is a renal cell carcinoma or melanoma.

21. The method of claim 18, wherein said cancer is a leukemia or a lymphoma.

22. The method of claim 13, wherein the subject has a non-neoplastic hyperproliferative disorder.

23. The method of claim 22, wherein the hyperproliferative disorder is an epithelial cell disorder.

24. The method of claim 23, wherein the hyperproliferative disorder is asthma, chronic pulmonary obstructive disease, lung fibrosis, bronchial hyper responsiveness, psoriasis, and seborrheic dermatitis.

25. The method of claim 1 or 13, wherein the EphA2 polypeptide comprises full length EphA2.

26. The method of any one of claims 1 and 13, wherein the EphA2 polypeptide comprises the extracellular domain of EphA2.

27. The method of any one of claims 1 and 13, wherein the EphA2 polypeptide is a chimeric polypeptide comprising at least an antigenic portion of EphA2 and a second polypeptide.

28. The method of claim 1 or 13, wherein the composition comprises a plurality of EphA2 antigenic peptide-expressing Listeria.

29. The method of claim 1 or 13, wherein the EphA2 antigenic peptide-expressing Listeria expresses a plurality of EphA2 antigenic peptides.

30. The method of any one of claims 1 and 13, further comprising administering an additional anti-cancer therapy.

31. The method of claim 30, wherein the additional anti-cancer therapy is an agonistic EphA2 antibody.

32. The method of claim 30, wherein the additional anti-cancer therapy is an anti-idiotype of an agonistic EphA2 antibody.

33. The method of claim 30, wherein the additional anti-cancer therapy is chemotherapy, biological-therapy, immunotherapy, radiation therapy, hormonal therapy, or surgery.

34. The method of any one of claims 1 and 13, wherein said administering is mucosal, parenteral, intramuscular, intraperitoneal, intravenous or oral.

35. The method of claim 1 or 13, wherein the administration elicits a CD4⁺ T-cell response, a CD8⁺ T-cell response, an innate immune response, an antibody response, or a combination of one or more of the foregoing.

36. The method of claim 35, wherein the administration elicits both a CD4⁺ T-cell response and a CD8⁺ T-cell response.

37. A method of treating a human subject having a disease involving aberrant angiogenesis, said method comprising administering to the subject a composition comprising an EphA2 antigenic peptide-expressing Listeria bacterium in an amount effective to treat disease involving aberrant angiogenesis.

38. The method of claim 1, wherein the subject has a disease involving aberrant angiogenesis.

39. The method of claim 37 or 38, wherein the disease is macular degeneration, diabetic retinopathy, retinopathy of prematurity, vascular restenosis, infantile hemangioma, verruca vulgaris, psoriasis, Kaposi's sarcoma, neurofibromatosis, recessive dystrophic epidermolysis bullosa, rheumatoid arthritis, ankylosing spondylitis, systemic lupus, psoriatic arthropathy, Reiter's syndrome, and Sjogren's syndrome, endometriosis, preeclampsia, atherosclerosis or coronary artery disease.


SEQUENCE LISTING (72 sequences)

SEQ ID NO: 1 - DNA, 3963 bp, Homo sapiens
  CDS (138)..(3068)
attaaggactcggggcaggaggggcagaagttgcgcgcaggccggcgggcgggagcggacaccgaggccggcgtgcaggcgtgcgggtgtgcgggagccgggctcggggggatcggaccgagagcgagaagcgcggcatggagctccaggcagcccgcgcctgcttcgccctgctgtggggctgtgcgctggccgcggccgcggcggcgcagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtatcttcattgagctcaagtttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccctccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcaaggtgcacgaattccagacgctgtccccggagggatctggcaacttggcggtgattggcggcgtggctgtcggtgtggtcctgcttctggtgctggcaggagttggcttctttatccaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatctgagcctcgacagggcctggagccccatcggccaagaatacttgaagaaacagagtggcctccctgctgtgccatgctgggccactggggactttatttatttctagttctttcctccccctgcaacttccgctgaggggtctcggatgacaccctggcctgaactgaggagatgaccagggatgctgggctgggccctctttccctgcgagacgcacacagctgagcacttagcaggcaccgccacgtcccagcatccctggagcaggagccccgccacagccttcggacagacatataggatattcccaagccgaccttccctccgccttctcccacatgaggccatctcaggagatggagggcttggcccagcgccaagtaaacagggtacctcaagccccatttcctcacactaagagggcagactgtgaacttgactgggtgagacccaaagcggtccctgtccctctagtgccttctttagaccctcgggccccatcctcatccctgactggccaaacccttgctttcctgggcctttgcaagatgcttggttgtgttgaggtttttaaatatatattttgtactttgtggagagaatgtgtgtgtgtggcagggggccccgccagggctggggacagagggtgtcaaacattcgtgagctggggactcagggaccggtgctgcaggagtgtcctgcccatgccccagtcggccccatctctcatccttttggataagtttctattctgtcagtgttaaagattttgttttgttggacatttttttcgaatcttaatttattattttttttatatttattgttagaaaatgacttatttctgctctggaataaagttgcagatgattcaaaccgaaaaaaaaaaaaaaaaaa

SEQ ID NO: 2 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 3 - protein, 12 aa, Homo sapiens
TLADFDPRVPRT

SEQ ID NO: 4 - protein, 9 aa, Homo sapiens
VLLLVLAGV

SEQ ID NO: 5 - protein, 9 aa, Homo sapiens
VLAGVGFFI

SEQ ID NO: 6 - protein, 9 aa, Homo sapiens
IMNDMPIYM

SEQ ID NO: 7 - protein, 9 aa, Homo sapiens
SLLGLKDQV

SEQ ID NO: 8 - protein, 9 aa, Homo sapiens
WLVPIGQCL

SEQ ID NO: 9 - protein, 9 aa, Homo sapiens
LLWGCALAA

SEQ ID NO: 10 - protein, 9 aa, Homo sapiens
GLTRTSVTV

SEQ ID NO: 11 - protein, 9 aa, Homo sapiens
NLYYAESDL

SEQ ID NO: 12 - protein, 9 aa, Homo sapiens
KLNVEERSV

SEQ ID NO: 13 - protein, 9 aa, Homo sapiens
IMGQFSHHN

SEQ ID NO: 14 - protein, 9 aa, Homo sapiens
YSVCNVMSG

SEQ ID NO: 15 - protein, 9 aa, Homo sapiens
MQNIMNDMP

SEQ ID NO: 16 - protein, 15 aa, Homo sapiens
EAGIMGQFSHHNIIR

SEQ ID NO: 17 - protein, 13 aa, Homo sapiens
PIYMYSVCNVMSG

SEQ ID NO: 18 - protein, 16 aa, Homo sapiens
DLMQNIMNDMPIYMYS

SEQ ID NO: 19 - DNA, 3105 bp, Artificial Sequence
  Description of Artificial Sequence  Fusion construct
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatctcgagctccaggcagcccgcgcctgcttcgccctgctgtggggctgtgcgctggccgcggccgcggcggcgcagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtatcttcattgagctcaagtttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccctccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcagggtgcacgaattccagacgctgtccccggagggatctggcaacttggcggtgattggcggcgtggctgtcggtgtggtcctgcttctggtgctggcaggagttggcttctttatccaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatc

SEQ ID NO: 20 - protein, 1035 aa, Artificial Sequence
  Description of Artificial Sequence  Predicted fusion protein
MKKIMLVFITLILVSLPIAQQTEAKDASAFNKENSISSMAPPASPPASPKTPIEKKHADLELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSRVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 21 - DNA, 1506 bp, Homo sapiens
cagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtatcttcattgagctcaagtttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccctccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcagggtgcacgaattccagacg

SEQ ID NO: 22 - DNA, 1506 bp, Artificial Sequence
  Description of Artificial Sequence  Human sequence optimized for codon usage in Listeria
caaggtaaagaagttgttttattagattttgcagcagcaggtggtgaattaggttggttaacacatccatatggtaaaggttgggatttaatgcaaaatattatgaatgatatgccaatttatatgtatagtgtttgtaatgttatgagtggtgatcaagataattggttacgtacaaattgggtttatcgtggtgaagcagaacgtatttttattgaattaaaatttacagttcgtgattgtaatagttttccaggtggtgcaagtagttgtaaagaaacatttaatttatattatgcagaaagtgatttagattatggtacaaattttcaaaaacgtttatttacaaaaattgatacaattgcaccagatgaaattacagttagtagtgattttgaagcacgtcatgttaaattaaatgttgaagaacgtagtgttggtccattaacacgtaaaggtttttatttagcatttcaagatattggtgcatgtgttgcattattaagtgttcgtgtttattataaaaaatgtccagaattattacaaggtttagcacattttccagaaacaattgcaggtagtgatgcaccaagtttagcaacagttgcaggtacatgtgttgatcatgcagttgttccaccaggtggtgaagaaccacgtatgcattgtgcagttgatggtgaatggttagttccaattggtcaatgtttatgtcaagcaggttatgaaaaagttgaagatgcatgtcaagcatgtagtccaggtttttttaaatttgaagcaagtgaaagtccatgtttagaatgtccagaacatacattaccaagtccagaaggtgcaacaagttgtgaatgtgaagaaggtttttttcgtgcaccacaagatccagcaagtatgccatgtacacgtccaccaagtgcaccacattatttaacagcagttggtatgggtgcaaaagttgaattacgttggacaccaccacaagatagtggtggtcgtgaagatattgtttatagtgttacatgtgaacaatgttggccagaaagtggtgaatgtggtccatgtgaagcaagtgttcgttatagtgaaccaccacatggtttaacacgtacaagtgttacagttagtgatttagaaccacatatgaattatacatttacagttgaagcacgtaatggtgttagtggtttagttacaagtcgtagttttcgtacagcaagtgttagtattaatcaaacagaaccaccaaaagttcgtttagaaggtcgtagtacaacaagtttaagtgttagttggagtattccaccaccacaacaaagtcgtgtttggaaatatgaagttacatatcgtaaaaaaggtgatagtaatagttataatgttcgtcgtacagaaggttttagtgttacattagatgatttagcaccagatacaacatatttagttcaagttcaagcattaacacaagaaggtcaaggtgcaggtagtcgtgttcatgaatttcaaaca

SEQ ID NO: 23 - protein, 502 aa, Homo sapeins
QGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSRVHEFQT

SEQ ID NO: 24 - DNA, 1689 bp, Artificial Sequence
  Description of Artificial Sequence  Fusion protein construct
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatctcgagcagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtatcttcattgagctcaagtttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccctccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcagggtgcacgaattccagacg

SEQ ID NO: 25 - protein, 563 aa, Artificial Sequence
  Description of Artificial Sequence  Predicted fusion protein
MKKIMLVFITLILVSLPIAQQTEAKDASAFNKENSISSMAPPASPPASPKTPIEKKHADLEQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSRVHEFQT

SEQ ID NO: 26 - DNA, 1989 bp, Artificial Sequence
  Description of Artificial Sequence  Fusion protein construct
ggtacctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatggatccgattataaagatgatgatgataaacaaggtaaagaagttgttttattagattttgcagcagcaggtggtgaattaggttggttaacacatccatatggtaaaggttgggatttaatgcaaaatattatgaatgatatgccaatttatatgtatagtgtttgtaatgttatgagtggtgatcaagataattggttacgtacaaattgggtttatcgtggtgaagcagaacgtatttttattgaattaaaatttacagttcgtgattgtaatagttttccaggtggtgcaagtagttgtaaagaaacatttaatttatattatgcagaaagtgatttagattatggtacaaattttcaaaaacgtttatttacaaaaattgatacaattgcaccagatgaaattacagttagtagtgattttgaagcacgtcatgttaaattaaatgttgaagaacgtagtgttggtccattaacacgtaaaggtttttatttagcatttcaagatattggtgcatgtgttgcattattaagtgttcgtgtttattataaaaaatgtccagaattattacaaggtttagcacattttccagaaacaattgcaggtagtgatgcaccaagtttagcaacagttgcaggtacatgtgttgatcatgcagttgttccaccaggtggtgaagaaccacgtatgcattgtgcagttgatggtgaatggttagttccaattggtcaatgtttatgtcaagcaggttatgaaaaagttgaagatgcatgtcaagcatgtagtccaggtttttttaaatttgaagcaagtgaaagtccatgtttagaatgtccagaacatacattaccaagtccagaaggtgcaacaagttgtgaatgtgaagaaggtttttttcgtgcaccacaagatccagcaagtatgccatgtacacgtccaccaagtgcaccacattatttaacagcagttggtatgggtgcaaaagttgaattacgttggacaccaccacaagatagtggtggtcgtgaagatattgtttatagtgttacatgtgaacaatgttggccagaaagtggtgaatgtggtccatgtgaagcaagtgttcgttatagtgaaccaccacatggtttaacacgtacaagtgttacagttagtgatttagaaccacatatgaattatacatttacagttgaagcacgtaatggtgttagtggtttagttacaagtcgtagttttcgtacagcaagtgttagtattaatcaaacagaaccaccaaaagttcgtttagaaggtcgtagtacaacaagtttaagtgttagttggagtattccaccaccacaacaaagtcgtgtttggaaatatgaagttacatatcgtaaaaaaggtgatagtaatagttataatgttcgtcgtacagaaggttttagtgttacattagatgatttagcaccagatacaacatatttagttcaagttcaagcattaacacaagaaggtcaaggtgcaggtagtcgtgttcatgaatttcaaacagaacaaaaattaattagtgaagaagatttatgagagctc

SEQ ID NO: 27 - protein, 581 aa, Artificial Sequence
  Description of Artificial Sequence  Predicted fusion protein
MKKIMLVFITLILVSLPIAQQTEAKDASAFNKENSISSMAPPASPPASPKTPIEKKHADGSDYKDDDDKQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSRVHEFQTEQKLISEEDL

SEQ ID NO: 28 - DNA, 1989 bp, Artificial Sequence
  Description of Artificial Sequence  Construct for fusion protein
ggtacctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaattatgttagtttttattacattaattttagttagtttaccaattgcacaacaaacagaagcaaaagatgcaagtgcatttaataaagaaaatagtattagtagtatggcaccaccagcaagtccaccagcaagtccaaaaacaccaattgaaaaaaaacatgcagatggatccgattataaagatgatgatgataaacaaggtaaagaagttgttttattagattttgcagcagcaggtggtgaattaggttggttaacacatccatatggtaaaggttgggatttaatgcaaaatattatgaatgatatgccaatttatatgtatagtgtttgtaatgttatgagtggtgatcaagataattggttacgtacaaattgggtttatcgtggtgaagcagaacgtatttttattgaattaaaatttacagttcgtgattgtaatagttttccaggtggtgcaagtagttgtaaagaaacatttaatttatattatgcagaaagtgatttagattatggtacaaattttcaaaaacgtttatttacaaaaattgatacaattgcaccagatgaaattacagttagtagtgattttgaagcacgtcatgttaaattaaatgttgaagaacgtagtgttggtccattaacacgtaaaggtttttatttagcatttcaagatattggtgcatgtgttgcattattaagtgttcgtgtttattataaaaaatgtccagaattattacaaggtttagcacattttccagaaacaattgcaggtagtgatgcaccaagtttagcaacagttgcaggtacatgtgttgatcatgcagttgttccaccaggtggtgaagaaccacgtatgcattgtgcagttgatggtgaatggttagttccaattggtcaatgtttatgtcaagcaggttatgaaaaagttgaagatgcatgtcaagcatgtagtccaggtttttttaaatttgaagcaagtgaaagtccatgtttagaatgtccagaacatacattaccaagtccagaaggtgcaacaagttgtgaatgtgaagaaggtttttttcgtgcaccacaagatccagcaagtatgccatgtacacgtccaccaagtgcaccacattatttaacagcagttggtatgggtgcaaaagttgaattacgttggacaccaccacaagatagtggtggtcgtgaagatattgtttatagtgttacatgtgaacaatgttggccagaaagtggtgaatgtggtccatgtgaagcaagtgttcgttatagtgaaccaccacatggtttaacacgtacaagtgttacagttagtgatttagaaccacatatgaattatacatttacagttgaagcacgtaatggtgttagtggtttagttacaagtcgtagttttcgtacagcaagtgttagtattaatcaaacagaaccaccaaaagttcgtttagaaggtcgtagtacaacaagtttaagtgttagttggagtattccaccaccacaacaaagtcgtgtttggaaatatgaagttacatatcgtaaaaaaggtgatagtaatagttataatgttcgtcgtacagaaggttttagtgttacattagatgatttagcaccagatacaacatatttagttcaagttcaagcattaacacaagaaggtcaaggtgcaggtagtcgtgttcatgaatttcaaacagaacaaaaattaattagtgaagaagatttatgagagctc

SEQ ID NO: 29 - protein, 581 aa, Artificial Sequence; identical to SEQ ID NO: 27
  Description of Artificial Sequence  Predicted Fusion protein

SEQ ID NO: 30 - DNA, 1968 bp, Artificial Sequence
  Description of Artificial Sequence  Fusion protein construct
ggtacctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatggcatacgacagtcgttttgatgaatgggtacagaaactgaaagaggaaagctttcaaaacaatacgtttgaccgccgcaaatttattcaaggagcggggaagattgcaggactttctcttggattaacgattgcccagtcggttggggcctttggatccgattataaagatgatgatgataaacaaggtaaagaagttgttttattagattttgcagcagcaggtggtgaattaggttggttaacacatccatatggtaaaggttgggatttaatgcaaaatattatgaatgatatgccaatttatatgtatagtgtttgtaatgttatgagtggtgatcaagataattggttacgtacaaattgggtttatcgtggtgaagcagaacgtatttttattgaattaaaatttacagttcgtgattgtaatagttttccaggtggtgcaagtagttgtaaagaaacatttaatttatattatgcagaaagtgatttagattatggtacaaattttcaaaaacgtttatttacaaaaattgatacaattgcaccagatgaaattacagttagtagtgattttgaagcacgtcatgttaaattaaatgttgaagaacgtagtgttggtccattaacacgtaaaggtttttatttagcatttcaagatattggtgcatgtgttgcattattaagtgttcgtgtttattataaaaaatgtccagaattattacaaggtttagcacattttccagaaacaattgcaggtagtgatgcaccaagtttagcaacagttgcaggtacatgtgttgatcatgcagttgttccaccaggtggtgaagaaccacgtatgcattgtgcagttgatggtgaatggttagttccaattggtcaatgtttatgtcaagcaggttatgaaaaagttgaagatgcatgtcaagcatgtagtccaggtttttttaaatttgaagcaagtgaaagtccatgtttagaatgtccagaacatacattaccaagtccagaaggtgcaacaagttgtgaatgtgaagaaggtttttttcgtgcaccacaagatccagcaagtatgccatgtacacgtccaccaagtgcaccacattatttaacagcagttggtatgggtgcaaaagttgaattacgttggacaccaccacaagatagtggtggtcgtgaagatattgtttatagtgttacatgtgaacaatgttggccagaaagtggtgaatgtggtccatgtgaagcaagtgttcgttatagtgaaccaccacatggtttaacacgtacaagtgttacagttagtgatttagaaccacatatgaattatacatttacagttgaagcacgtaatggtgttagtggtttagttacaagtcgtagttttcgtacagcaagtgttagtattaatcaaacagaaccaccaaaagttcgtttagaaggtcgtagtacaacaagtttaagtgttagttggagtattccaccaccacaacaaagtcgtgtttggaaatatgaagttacatatcgtaaaaaaggtgatagtaatagttataatgttcgtcgtacagaaggttttagtgttacattagatgatttagcaccagatacaacatatttagttcaagttcaagcattaacacaagaaggtcaaggtgcaggtagtcgtgttcatgaatttcaaacagaacaaaaattaattagtgaagaagatttatgagagctc

SEQ ID NO: 31 - protein, 574 aa, Artificial Sequence
  Description of Artificial Sequence  Predicted Fusion Protein
MAYDSRFDEWVQKLKEESFQNNTFDRRKFIQGAGKIAGLSLGLTIAQSVGAFGSDYKDDDDKQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSRVHEFQTEQKLISEEDL

SEQ ID NO: 32 - DNA, 1254 bp, Homo sapiens
caccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatc

SEQ ID NO: 33 - DNA, 1254 bp, Artificial Sequence
  Description of Artificial Sequence  Sequence Optimized for codon usage in Listeria
cacagacgtagaaaaaatcaacgtgctcgacaatccccagaagatgtgtatttttcgaaaagtgaacaattaaaaccattaaaaacttatgttgatccgcatacgtacgaagacccaaatcaagcagtattaaaatttacaacagaaatacacccaagttgtgttacaagacaaaaagttattggagcaggtgaattcggagaggtatataaaggtatgttaaaaacatcatcaggtaaaaaagaagttccggttgcaattaaaaccttaaaggcaggatatacagaaaaacagcgagttgattttttaggtgaagcaggaattatgggtcaatttagccatcataatattattcgtttggaaggagtaataagtaaatataaaccaatgatgattattacagaatacatggaaaacggtgctttagataaatttttacgtgaaaaggatggtgaatttagtgttttacaattggttggtatgttaagaggaattgctgcaggtatgaaatatttagctaatatgaattatgttcaccgtgatttggcagcaagaaatatcctagtcaattccaatttagtatgtaaagttagtgattttggtttaagcagagtattagaagacgatccagaggcaacctatacaacatcgggaggtaaaattcctattcgttggacagcaccagaagctatcagttaccgtaaatttacaagtgcatcagacgtgtggagttttgggattgtaatgtgggaagttatgacatatggagaaagaccatattgggaattaagtaatcatgaagttatgaaagcaattaacgatggatttagattaccaactccgatggattgtccatctgccatttatcaactaatgatgcaatgttggcaacaagaaagagcacgacgtccaaaatttgcagatattgttagtattttagacaaattaattcgtgcaccagatagtttaaaaactttagcagactttgatcctcgtgttagtattcgattaccaagtacgtcaggttccgaaggagttccatttcgcacagtctccgaatggttggaatcaattaaaatgcaacaatacaccgaacactttatggcagcaggttacacagcaatcgaaaaagttgttcaaatgacaaatgatgatattaaacgtattggagttagattaccaggccaccagaaacgtattgcatattctttattaggtttaaaagatcaagttaataccgtgggaattccaatt

SEQ ID NO: 34 - protein, 456 aa, Homo sapiens
VHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 35 - DNA, 1437 bp, Artificial Sequence
  Description of Artificial Sequence  Fusion Protein
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatctcgagcaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatc

SEQ ID NO: 36 - protein, 479 aa, Artificial Sequence
  Description of Artificial Sequence  Predicted Protein Sequence
MKKIMLVFITLILVSLPIAQQTEAKDASAFNKENSISSMAPPASPPASPKTPIEKKHADLEHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 37 - DNA, 1737 bp, Artificial Sequence
  Description of Artificial Sequence  Fusion protein sequence
ggtacctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatggatccgattataaagatgatgatgataaacacagacgtagaaaaaatcaacgtgctcgacaatccccagaagatgtgtatttttcgaaaagtgaacaattaaaaccattaaaaacttatgttgatccgcatacgtacgaagacccaaatcaagcagtattaaaatttacaacagaaatacacccaagttgtgttacaagacaaaaagttattggagcaggtgaattcggagaggtatataaaggtatgttaaaaacatcatcaggtaaaaaagaagttccggttgcaattaaaaccttaaaggcaggatatacagaaaaacagcgagttgattttttaggtgaagcaggaattatgggtcaatttagccatcataatattattcgtttggaaggagtaataagtaaatataaaccaatgatgattattacagaatacatggaaaacggtgctttagataaatttttacgtgaaaaggatggtgaatttagtgttttacaattggttggtatgttaagaggaattgctgcaggtatgaaatatttagctaatatgaattatgttcaccgtgatttggcagcaagaaatatcctagtcaattccaatttagtatgtaaagttagtgattttggtttaagcagagtattagaagacgatccagaggcaacctatacaacatcgggaggtaaaattcctattcgttggacagcaccagaagctatcagttaccgtaaatttacaagtgcatcagacgtgtggagttttgggattgtaatgtgggaagttatgacatatggagaaagaccatattgggaattaagtaatcatgaagttatgaaagcaattaacgatggatttagattaccaactccgatggattgtccatctgccatttatcaactaatgatgcaatgttggcaacaagaaagagcacgacgtccaaaatttgcagatattgttagtattttagacaaattaattcgtgcaccagatagtttaaaaactttagcagactttgatcctcgtgttagtattcgattaccaagtacgtcaggttccgaaggagttccatttcgcacagtctccgaatggttggaatcaattaaaatgcaacaatacaccgaacactttatggcagcaggttacacagcaatcgaaaaagttgttcaaatgacaaatgatgatattaaacgtattggagttagattaccaggccaccagaaacgtattgcatattctttattaggtttaaaagatcaagttaataccgtgggaattccaattgaacaaaaattaatttccgaagaagacttataagagctc

SEQ ID NO: 38 - protein, 497 aa, Artificial Sequence
  Description of Artificial Sequence  Predicted fusion protein
MKKIMLVFITLILVSLPIAQQTEAKDASAFNKENSISSMAPPASPPASPKTPIEKKHADGSDYKDDDDKHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPIEQKLISEEDL

SEQ ID NO: 39 - DNA, 1737 bp, Artificial Sequence
  Description of Artificial Sequence  Fusion protein construct
ggtacctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaattatgttagtttttattacattaattttagttagtttaccaattgcacaacaaacagaagcaaaagatgcaagtgcatttaataaagaaaatagtattagtagtatggcaccaccagcaagtccaccagcaagtccaaaaacaccaattgaaaaaaaacatgcagatggatccgattataaagacgatgatgataaacacagacgtagaaaaaatcaacgtgctcgacaatccccagaagatgtgtatttttcgaaaagtgaacaattaaaaccattaaaaacttatgttgatccgcatacgtacgaagacccaaatcaagcagtattaaaatttacaacagaaatacacccaagttgtgttacaagacaaaaagttattggagcaggtgaattcggagaggtatataaaggtatgttaaaaacatcatcaggtaaaaaagaagttccggttgcaattaaaaccttaaaggcaggatatacagaaaaacagcgagttgattttttaggtgaagcaggaattatgggtcaatttagccatcataatattattcgtttggaaggagtaataagtaaatataaaccaatgatgattattacagaatacatggaaaacggtgctttagataaatttttacgtgaaaaggatggtgaatttagtgttttacaattggttggtatgttaagaggaattgctgcaggtatgaaatatttagctaatatgaattatgttcaccgtgatttggcagcaagaaatatcctagtcaattccaatttagtatgtaaagttagtgattttggtttaagcagagtattagaagacgatccagaggcaacctatacaacatcgggaggtaaaattcctattcgttggacagcaccagaagctatcagttaccgtaaatttacaagtgcatcagacgtgtggagttttgggattgtaatgtgggaagttatgacatatggagaaagaccatattgggaattaagtaatcatgaagttatgaaagcaattaacgatggatttagattaccaactccgatggattgtccatctgccatttatcaactaatgatgcaatgttggcaacaagaaagagcacgacgtccaaaatttgcagatattgttagtattttagacaaattaattcgtgcaccagatagtttaaaaactttagcagactttgatcctcgtgttagtattcgattaccaagtacgtcaggttccgaaggagttccatttcgcacagtctccgaatggttggaatcaattaaaatgcaacaatacaccgaacactttatggcagcaggttacacagcaatcgaaaaagttgttcaaatgacaaatgatgatattaaacgtattggagttagattaccaggccaccagaaacgtattgcatattctttattaggtttaaaagatcaagttaataccgtgggaattccaattgaacaaaaattaatttccgaagaagacttataagagctc

SEQ ID NO: 40 - protein, 497 aa, Artificial Sequence; identical to SEQ ID NO: 38
  Description of Artificial Sequence  Predicted Fusion Protein

SEQ ID NO: 41 - DNA, 1716 bp, Artificial Sequence
  Description of Artificial Sequence  Fusion protein construct
ggtacctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatggcatacgacagtcgttttgatgaatgggtacagaaactgaaagaggaaagctttcaaaacaatacgtttgaccgccgcaaatttattcaaggagcggggaagattgcaggactttctcttggattaacgattgcccagtcggttggggcctttggatccgattataaagatgatgatgataaacacagacgtagaaaaaatcaacgtgctcgacaatccccagaagatgtgtatttttcgaaaagtgaacaattaaaaccattaaaaacttatgttgatccgcatacgtacgaagacccaaatcaagcagtattaaaatttacaacagaaatacacccaagttgtgttacaagacaaaaagttattggagcaggtgaattcggagaggtatataaaggtatgttaaaaacatcatcaggtaaaaaagaagttccggttgcaattaaaaccttaaaggcaggatatacagaaaaacagcgagttgattttttaggtgaagcaggaattatgggtcaatttagccatcataatattattcgtttggaaggagtaataagtaaatataaaccaatgatgattattacagaatacatggaaaacggtgctttagataaatttttacgtgaaaaggatggtgaatttagtgttttacaattggttggtatgttaagaggaattgctgcaggtatgaaatatttagctaatatgaattatgttcaccgtgatttggcagcaagaaatatcctagtcaattccaatttagtatgtaaagttagtgattttggtttaagcagagtattagaagacgatccagaggcaacctatacaacatcgggaggtaaaattcctattcgttggacagcaccagaagctatcagttaccgtaaatttacaagtgcatcagacgtgtggagttttgggattgtaatgtgggaagttatgacatatggagaaagaccatattgggaattaagtaatcatgaagttatgaaagcaattaacgatggatttagattaccaactccgatggattgtccatctgccatttatcaactaatgatgcaatgttggcaacaagaaagagcacgacgtccaaaatttgcagatattgttagtattttagacaaattaattcgtgcaccagatagtttaaaaactttagcagactttgatcctcgtgttagtattcgattaccaagtacgtcaggttccgaaggagttccatttcgcacagtctccgaatggttggaatcaattaaaatgcaacaatacaccgaacactttatggcagcaggttacacagcaatcgaaaaagttgttcaaatgacaaatgatgatattaaacgtattggagttagattaccaggccaccagaaacgtattgcatattctttattaggtttaaaagatcaagttaataccgtgggaattccaattgaacaaaaattaatttccgaagaagacttataagagctc

SEQ ID NO: 42 - protein, 490 aa, Artificial Sequence
  Description of Artificial Sequence  Predicted fusion protein
MAYDSRFDEWVQKLKEESFQNNTFDRRKFIQGAGKIAGLSLGLTIAQSVGAFGSDYKDDDDKHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPIEQKLISEEDL

SEQ ID NO: 43 - DNA, 9808 bp, Artificial Sequence
  Description of Artificial Sequence  Fusion Protein Construct
ctttaaacgtggatcattttctttaaatttatgctgacgacctttgaatttgccttttttcttagcaatttcgattccttgtgcctgacgttccttaattttttttcgttctgattctgcttgatacttgtacaattcaatgacaaggctattaatcaaacgccttaaattttcatcttcaataccattcattgagggtaaatttaagacttccagggttgcccccttaatttgaatttgattcatcaattctgttaattctttattatttcgtcctaatcgatctaattcagtaacaataacaatatccccttcacgaatatagttaagcatagcttgtaattgtgggcgttcgaccgattgaccgcttaatttgtctgaaaagaccttagaaacgccctgtaacgcttgtaattgccgatctaagttctgttctttgctactgacacgtgcataaccaattttagccattttcaaccaacctctaaaattctctcggttgcaataaccaatcagcaatatctactttttcaatttcaaattgcttatcagaaattgtcttttcgtaagcgataaaatcttgcgcatattgttgctcattaaaaatagccaccacttcgtcattttctaaaactcgataaataaattttttcattttactcctcctattatgcccaacttaaatgacctattcaccaagtcaattatactgctaaaatcatattaggacaaataggtatactctattgacctataaatgatagcaacttaaaagatcaagtgttcgcttcgctctcactgcccctcgacgttttagtagcctttccctcacttcgttcagtccaagccaactaaaagttttcgggctactctctccttctccccctaataattaattaaaatcttactctgtatatttctgctaatcattcactaaacagcaaagaaaaacaaacacgtatcatagatataaatgtaatggcatagtgcgggttttattttcagcctgtatcgtagctaaacaaatcgagttgtgggtccgttttggggcgttctgccaatttgtttagagtttcttgaataaatgtacgttctaaattaaacgaagctgtcagcgcctttatatagctttctcgttcttctttttttaatttaatgatcgatagcaacaatgatttaacactagcaagttgaatgccaccatttcttcctggtttaatcttaaagaaaatttcctgattcgccttcagtaccttcagcaatttatctaatgtccgttcaggaatgcctagcacttctctaatctcttttttggtcgtcgctaaataaggcttgtatacatcgcttttttcgctaatataagccattaaatcttctttccattctgacaaatgaacacgttgacgttcgcttctttttttcttgaatttaaaccacccttgacggacaaataaatctttactggttaaatcacttgatacccaagctttgcaaagaatggtaatgtattccctattagccccttgatagttttctgaataggcacttctaacaattttgattacttctttttcttctaagggttgatctaatcgattattaaactcaaacatattatattcgcacgtttcgattgaatagcctgaactaaagtaggctaaagagagggtaaacataacgctattgcgccctactaaacccttttctcctgaaaatttcgtttcgtgcaataagagattaaaccagggttcatctacttgttttttgccttctgtaccgcttaaaaccgttagacttgaacgagtaaagcccttattatctgtttgtttgaaagaccaatcttgccattctttgaaagaataacggtaattgggatcaaaaaattctacattgtccgttcttggtatacgagcaatcccaaaatgattgcacgttagatcaactggcaaagactttccaaaatattctcggatattttgcgagattattttggctgctttgacagatttaaattctgattttgaagtcacatagactggcgtttctaaaacaaaatatgcttgataacctttatcagatttgataattaacgtaggcataaaacctaaatcaatagctgttgttaaaatatcgcttgctgaaatagtttctttttccgtgtgaatatcaaaatcaataaagaaggtattgatttgtcttaaattgttttcagaatgtcctttagtgtatgaacggttttcgtctgcatacgtaccataacgataaacgtttggtgtccaatgcgtaaatgtatcttgattttcgtgaatcgcttcttcggaagtcagaacaacgccacgtccgccaatcatgcttttttttgagcgatacgcaaaaatagcccctttacttttacctggcttggtagtgattgagcgaattttactatttttaaatttgtactttaacaagccgtcatgaagcacagtttctacaacaaaagggatattcattcagctgttctcctttcttacgaaaattaattagttagaagctacgatcaaagttgaatcacaacaaaaaaggcaatcaactaagtttttcttaattgattgcctggtatcttcttaaagacttgaaatcccctcaaaaacccgatataatgggtttacagatatttaagtatctgattaataaagtaattaaatactttaccaaattttgggtctcgacttctttaattgattggtggtaatcaattaaggctcgcagttaaaatttctcaggctttaactggtcgtggctctttttttgtattctttattcagttcgttgtttcgttatatctagtatatcgctttttaaaaaaataagcaatgatttcgtgcattattcacacgaaatcattgcttttttcttcttccatttctaactccaatgttacttgttctgtttctggttctggttctgttggctcatttgggattaaatccactactagcgttgagttagttccgtctctaatagccggttaagtaatagccggttaagtggtcaaactttgggaaaatctcaacccgcattaagttttgatgccatgacaatcgttggaaatttgaacaaaactaatgctaaaaagctatctgactttatgagtgtagagccacaaatacgactttgggatatacttcaaacaaagtttaaagctaaggcacttcaagaaaaagtttatatcgaatatgacaaagtaaaagcagatacttgggatagacgtaatatgcgtgttgaatttaatcccaataaactcacacatgaagaaatgatttggttaaaacaaaatattatcgactacatggaagatgacggttttacaagattagacttagcttttgattttgaagatgatttgagcgattactatgcaatgactgataaagcagttaagaaaactgttttttatggtcgtaatggcaagccagaaacaaaatattttggtgtccgtgatagtgatagatttattagaatttataataaaaaacaagaacgtaaagataacgcagatgttgaagttgtgtttgaacatttatggcgtgtagaagttgaattaaaaagagatatggttgattactggaatgattgttttaatgatttacacatctttgaaacctgcgtgggctactttagaaaaaattaatgagcaagctatggtttatactttgttgcatgaagaaagtatgtggggaaagctaagtaagaatactaagactaaatttaaaaaattgattagagaaatatctccaattgatttaacggaattaatgaaatcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcataaaatttatgaacgtataacaaccacattttttggttgcttgtggttttgattttgaatttggttttgaacttatggactgatttattcagtccattttttgtgcttgcacaaaaactagcctcgcagagcacacgcattaatgacttatgaaacgtagtaaataagtctagtgtgttatactttacttggaagatgcaccgaataaaaaatattgaagaacaactagcaaaagattttaaagagttattttattttaagtctttataacatgagtgaagcgaatttttaaatttcgatagaaatttttacatcaaaaagccccctgtcaaaattgacgaagggggttttttggcgcacgcttttcgttagaaatatacaagattgaaaatcgtgtataagtgcgccctttgttttgaacttagcacgttacatcaattttttaaaatgatgtataagtgcgcccttttaaattttgagtgattatattttttgagttagaaaaagggattgggaaaatttcccaaaataatttaaaaaataagcaaaaattttcgatagagaatgtgctattttttgtcaaaggtgtataccttgactgtgcttgctgttacattaagtttatttttaagttattaaaaaagaaatagcttttaaagtttggctcgctgtcgctttataaagctgattgacttttgattgcaaactacttaaagaaaacaaactcggactattcgttttcttctctttggtttgaacatcagcaattatcccctcttgattgcctattttagcttgtttagaagaaacaaaagctaaaagctcctcttgggttttaaaacgctgtgtggggcttagaacgcccttaaacgacccttggtttacttttatactagcttccacctcgaaaaaaggttcttttttaaaattctctatggcttcctggcgctgaaaaaataaggtataaggtgggcgtttgaacacgtcctagtgaaaatgtaccttgtacgccccttctgttgtaaatttaacgtatacaaagggcttgcgttcatgccgatcaaccaatcggcaatttggcgtgtttgcgcttcttgataaaagggatagtaattcattccaggttgcaaattttgaaaaccgcttcggattacatctttttctaagctattgatccatagtcttttaaatgttttatcttttgaaaaggcatttgctttatggataatcgaccaggcgatattttcaccttctctgtcgctatctgttgcaacaataattgtatttgcctttttgagaagttctgcaacaattttaaactgctttcccttatcttttgcaacttcaaaatcgtatcgatcaggaaaaatcggcaaagattcaagtttccaattttgccacttttcgtcataatgacctggttctgctaattccactaaatgcccaaaaccaaaggtgataaacgtttcatctgtaaatagtgggtctttgatctcaaaataaccgtcttttttggtgctttgttttaaagcacttgcgtaggctaatgcctggcttggtttttcagctaaaataaccgtactcattaactatccctcttttcattgttttttctttgatcgactgtcacgttatatcttgctcgataccttctaaacgttcggcgattgattccagtttgttcttcaacttctttatcggataaaccattcaaaaacaaatcgaaagcatggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaatgctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccga

SEQ ID NO: 44 - protein, 26 aa, Listeria monocytogenes
MKKIMLVFITLILVSLPIAQQTEAKD

SEQ ID NO: 45 - protein, 59 aa, Listeria monocytogenes
MTDKKSENQTEKTETKENKGMTRREMLKLSAVAGTGIAVGATGLGTILNVVDQVDKALT

SEQ ID NO: 46 - protein, 53 aa, Bacillus subtillus
MAYDSRFDEWVQKLKEESFQNNTFDRRKFIQGAGKIAGLSLGLTIAQSVGAFG

SEQ ID NO: 47 - DNA, 21 bp, Artificial Sequence
  Description of Artificial Sequence  Primer
gtcaaaacatacgctcttatc

SEQ ID NO: 48 - DNA, 24 bp, Artificial Sequence
  Description of Artificial Sequence  Primer
acataatcagtccaaagtagatgc

SEQ ID NO: 49 - DNA, 29 bp, Artificial Sequence
  Description of Artificial Sequence  Primer
ctctggtacctcctttgattagtatattc

SEQ ID NO: 50 - DNA, 29 bp, Artificial Sequence
  Description of Artificial Sequence  Primer
ctctggatccatccgcgtgtttcttttcg

SEQ ID NO: 51 - DNA, 24 bp, Artificial Sequence
  Description of Artificial Sequence  Epitope insert
gattataaagatgatgatgataaa

SEQ ID NO: 52 - protein, 8 aa, Artificial Sequence
  Description of Artificial Sequence  Epitope
DYKDDDDK

SEQ ID NO: 53 - DNA, 30 bp, Artificial Sequence
  Description of Artificial Sequence  Epitope insert
gaacaaaaattaattagtgaagaagattta

SEQ ID NO: 54 - protein, 10 aa, Artificial Sequence
  Description of Artificial Sequence  Epitope
EQKLISEEDL

SEQ ID NO: 55 - protein, 9 aa, Mus sp.
SPSYVYHQF

SEQ ID NO: 56 - protein, 9 aa, Artificial Sequence
  Description of Artificial Sequence  Epitope
SPSYAYHQF

SEQ ID NO: 57 - DNA, 29 bp, Artificial Sequence
  Description of Artificial Sequence  Primer
ctctggtacctcctttgattagtatattc

SEQ ID NO: 58 - DNA, 36 bp, Artificial Sequence
  Description of Artificial Sequence  Primer
caatggatccctcgagatcataatttacttcatccc

SEQ ID NO: 59 - DNA, 32 bp, Artificial Sequence
  Description of Artificial Sequence  Primer
atttctcgagtccatggggggttctcatcatc

SEQ ID NO: 60 - DNA, 25 bp, Artificial Sequence
  Description of Artificial Sequence  Primer
ggtgctcgagtgcggccgcaagctt

SEQ ID NO: 61 - DNA, 37 bp, Artificial Sequence
  Description of Artificial Sequence  Primer
cgattcccctagttatgtttaccaccaatttgctgca

SEQ ID NO: 62 - DNA, 31 bp, Artificial Sequence
  Description of Artificial Sequence  Primer
gcaaattggtggtaaacataactaggggaat

SEQ ID NO: 63 - DNA, 27 bp, Artificial Sequence
  Description of Artificial Sequence  Epitope insert
agtccaagttatgcatatcatcaattt

SEQ ID NO: 64 - DNA, 33 bp, Artificial Sequence
  Description of Artificial Sequence  Primer
cgatagtccaagttatgcatatcatcaatttgc

SEQ ID NO: 65 - DNA, 34 bp, Artificial Sequence
  Description of Artificial Sequence  Primer
gtcgcaaattgatgatatgcataacttggactat

SEQ ID NO: 66 - RNA, 8 nt, Artificial Sequence
  Description of Artificial Sequence  Consensus Sequence
naggaggu

SEQ ID NO: 67 - DNA, 19 bp, Listeria monocytogenes
aaggagagtgaaacccatg

SEQ ID NO: 68 - DNA, 240 bp, Listeria monocytogenes
ggtacctcctttgattagtatattcctatcttaaagtgacttttatgttgaggcattaacatttgttaacgacgataaagggacagcaggactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatg

SEQ ID NO: 69 - DNA, 240 bp, Listeria monocytogenes
ggtacctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatg

SEQ ID NO: 70 - protein, 5 aa, Listeria monocytogenes
TEAKD

SEQ ID NO: 71 - protein, 5 aa, Listeria monocytogenes
DKALT

SEQ ID NO: 72 - protein, 5 aa, Bacillus subtillus
VGAFG